SEQUENCE LISTING (105 sequences)

SEQ ID NO: 1 - DNA, 1772 bp, Zea mays
  source 1..1772: mol_type = genomic DNA
gtacgtaacgaatctacgttaagatcaatctcttcgtggagattcggatccttttacttctagacgagggaaggaaagtttccctgggtcgtgacagttagttgggccgtgtactgggtgtagaatgtcaggcgcgttccaagtatcgctaaagcccaagtggcaggctgatcgatgcatcttggcccattagtcggataggtggttgctcgtcctagaccacaatggtccagcagtccatgaaaccatcaggcccacaagtttgttcccaccgaaaatagtttccgctccgcctccgagcccgcccggcccggctgcccgccaccgccgatctcccccaccgcaacccaccttgagacctgagcgagcgtcaccccgacgcgcgggtcgaccccgaagccgccggcgagccacagctagcatacgcggacgatccggggactctgctcgcctcgtgattgctgctggcttgccggtggtgtcatcggtacgtgttctgtatgaccgaattcctcgttggcggcagcagcagagaatgcagggtggtgccgcgtatgtagcagcgcttgctggttgcgccgccgcacagcagctgagcgcctgactgggagcctcagatcctacgcgcatgtatgcatgtgacgcgccatgcattaaccccatcagagctcagggtttcagctttcacttcaggagtcaggatcagaattcagaaagggtttcagttgagcctacaacctgtcaacctcctatatacatacatagggcttgcttcgttcgctcgtcgtcgatggacagacagatgcggtatgccgctcatgcatgttagctgttgttgtgtgccctccatcagttcgattatggatttttaagtgggcatgcagcatgtacgcgtgtacgtacgcggcgccgtgacgacgccgcgccccgaccgcgaccggccagcaggccgagacgctcgccgcctgattcgcgcgcgcgccatgcccatgctgtgttgctagctgctcaaactcacatgcctccgatccggagtccggctagcacctagcagcgagcttcgctcacattccttgcctgcgtgcggtgcgtgcagtacagcgactgcagcgagtttcgttcttcagaattcgccgactactggatcgattcatagtttcagacttaagaagaaaggactaaactgaaagggatgcgatgtgccctctcaaggactgaagcgtcctttttctctcaaacgacgacaggaggggggccgcgccggccgggccagcgtgcgtgctcatcgccgccgcagcagggatcggagacgggagggaccgccccacctgcctggcaagctcacggcgattacagttgcgttacagggaggatcgatggcagctgcagggcgctgtgatgcatatgctgatgcctgcacagtggcggagttatgtgtgcgggaacaaacgaacaggacgccgggaggccaggccagcgtgctctcgctaacggaggcaggcgcagcgcaaggacctcatccatccacccctccacaccgtgcccaccaccaccggttctggcagagtttcaggcagctgcaactgcatgcaatgcaatgcagcagcagggagcgccggcagtgccttcattttttttttgtgctccacccccacagctccactgcatggtgcgtggcgcatgccatggaagcactacgacgcaacactggtcggcgtaaacacatgcacgaggaagaggtggaggagagggcttgctttttcacgtgacac

SEQ ID NO: 2 - DNA, 1961 bp, Zea mays
  source 1..1961: mol_type = genomic DNA
gcaaatatgacgaagttaaaacgtgaaatcaagccactgggatacatggacatttgagatcagtccagatctctctctctttttttgctgcccagtgcttgctgcagttccaaccattatgctgtcatatgttcatatattgagttgtgatgttcatgtatcatgaatacacaacagtgctaaaatgtttagaaatcagtctagctggtaggaagcttaatggaaattcatcatatcttccctgcgacctgaatttggggcttccaaactacctgaagttggttacagattctagattactgtttggtgatattaggactggaactctattctggcttgtagcttctcaagtcattggaagtgatttagacaaggcctagatttttgtttgttggaccaatttttaaatctgaagctttgtgcaattaaagcagataatgtacataggcagcaggttagtttagctacgcaggctgccattccaaacacatcgttgcagtgaagcatcaaatctaattaaaacatgtatgataaacatgtcatagcttacctatgccaataagcagaatatttctcaactatatctataggttgctgcaagcaatattgaatttgaaggaacttgcccaagtttcattttataacacattgaggtgtcatgtcgattaaattaaatggagttgccaattcatatcacatttttttattgtacctccacttagcacttttgcaatactttcaagaacgttaggtcttttggtgggtggtggtaatatactcgtcttcagggggatgccacatgaagaaacaaatgcagtccttttagcttcattgttagaggctttccagtccttttgagatgagctcttcaatacactgcttgtactttatatcatttgatcttaaatactttgtgacaaaccccctgttcttccaatttaatctggttaagtgcagtagagcatctctctgcatgactgttgcataagttttgctggttcttgtgagttcatttcatagcaggataatgcaataccaaagtgatgcttgtatacattatactggcatactgcatccactcttcctatataagcaacataaattaattaccctagggggttcatctgttgcccattgggcttgccttgactatcggcaatcgttcctctagcagcagaatggggaaccccttaatggcattgaaaacggtaccctttagtggatattgagattttacttttaagcttttgggcattgtattcaatcactgatatttccataaaaaggataaccacaggttgaactttacaatggggtttgcagttactagcataatatggccctgttaagcggacaagtgctggtaaatttttatttccatttgccacacataattcttagttctatttatttactatattagcttttcttgtgttgcttatggctggatacaagctcagcgtgatgttagtaactggcctggtcattagcaatcgtatggaaaccagggacatggaataggaactctcttttgtgaacaatagctgctgtcagcctgtcagttttactctgtgcgcatctaatgcatgatagattgaaacacatcggatgtgatgtttaaggaatcttttgtatcggatctggatcatatggtcacaaccttcccactgtaaaacacgtcttgttagcttttctgcagtcttgtctgataattccatttgcatgccatgacaaccgacaacatgaacacaaacgacataataataatggcaatagtgcatctatacctagtctcttttggcttgccaatgtccatttgtgtttggtcaatacctcggagggactcccaacacgtatattctatgaagttcatcttcagagttatgttatgcccccctcccaccccccaatgaaacttgacaactacatttttttggtggctctgtatgatgaagctgtta

SEQ ID NO: 3 - DNA, 1763 bp, Zea mays
  source 1..1763: mol_type = genomic DNA
tgtagggaataatatcattgtgtcttccctagttcgtagtcctgaaatggtcatcagccaccaaagattattcactttgaaccaactgttttttttataaaaaaagagccacaaatcgagatgccgaattcaccaatgcgctacgttttcagtgacactgaggaacagtttatcagggagcgaggggggaaacgccgaaacggcatcggaatcggaatgagaagcagaatggggatttgaatgctgcggcctgcgagagggagcggagtggaatggaatgtcccccgcaggtgccacccctacgagccgtccaaaaccaggtcgaaatttacgccgccaaaagggccacgaaatccagaaaaccccagaggaataaagggctctccgcttggggggccgggtgatgagaggtggatatatatcttttcatgtcggtttcggcctctgtgggtaagagtggtggggggtgacgctaatctccacgcgcgcgcggctcgctcgcgtccggtccggcgctccgctacgggatgcatgtgcgagtgtatgcctgctgcctgcatgcgagattccgacggcgacgcaagagttatctggaactcgacggccgcagctagccggcggcccaaccagttctttctccggagatgacagcaaaaagacctggattcggatgcttcacggccgggggctgtcactccccgctctccccggccggtgatgcgatcgaagagagcagagcaatccaacgggcctgtccatttccaaagcatttccaaagcaaattccgagacatacatatatgccggcgggggccaagttgcatgcgctcgcgtggcgtggcgtggcgctttcgaatttcgatccggagcaggacgacgacgagccttgctcttgctatcgagcacgacgatatattccatgcagcagcaaaatatgctaacccacattaacaggctaggcacagcaggtttagcatggctctgtatgtaatacgtaaaatggacagccatggttttggtttcgctagcttcgaggtcaaaagggtattaacataattgaccttttaatcacgtacgtacgtcgaccatggccaggctggattcgaaaaaaaaagaggctacttacggtatatatgtgtataaactatgccacaaaaaaaaactttgctttgcagggggcttaatgggggagggaatatatctgaaagggacggagcgacagacgggagagattgcgtaagggctaagtgacgcctttcgacggcgagctttttttgggggggctgcagttgtgttgagagcaacgtcagacagacagagagtaacggatggcgataacgaactcctcggaacagtgtagtgtagcggcagggatggtttttggaaagctgggtttttttttcttttcttctggaaacaaattaaggccggcatgctatgctactgagtgctggccaggccatcagtgacggatgcacaggaggagcacctagtcctacgcaggaggacagatcgagccccggccggctggttgcacgtacttattactactagtgatataaaagcgaggagggacaggatctaacatctaattgcctgtacgtactgaatatatgtcttggtcgtcctggtgtggagttgaccagaggccggcgacatcatgtgacgcaattgtccagcctgaaaaggccaacattatcaacactgcataccttttctgccgttggtaacaaacaaaagaataatactgtagtagttttttta

SEQ ID NO: 4 - DNA, 2026 bp, Zea mays
  source 1..2026: mol_type = genomic DNA
acatatcatatcgtcatcccatagtacaatgatttgctcaaaaagcctaggctttaaggacaagttttgacacataaatttatcgacgacaaaattttatgcccacaaacatgtgagagaaacaaaggaacatgctaaaagatagaatacatgttatcaaacatttttcaagatttcataatttaggagatatacatgcgacttatcaaaatctctacctgctactggcttgtgtttttctataaaaagtattctcgactatttcctttcctacgactgactcccacctggtagataccctcattcactttattggacaattgagtttttctttttgtcatgaagaccaagaccttgttaccctttaatattaagtaaggaataaacatccttcaagaaaataaaaccacctataaaaacttcttcaatattcgttgaagtatttcttcattaaagttcaggtattctaaaggtttggagaagttctacaaaaaatgtatataaaatggtgcaacgtgattcttgtagaaatctctccattgtctttcttaattttgaatggaacaaggactcaaaatttgagagtaattaagtatatgggagaagcttatgtgctagcagattaggagtcatttttttgcttagagtattaaaatgtgaagtaagtcatctaactcatgaatcattgtaccatgtgcacatgtagtgcctaacacctatgcgatgtagacatgggatgttaaacctagagtagtcatagtgtgactttgttttcgcaaaaaaacaactaacattactagggccagattggcggtaagaaagactacaccatcaaaatttagaaaggtcatgcatagattgtatcacaaaaatactagatgttctatgattaaaaaacgttccaagaacgtggtgtctttgttctttgacaagctattgggcctgtacaggatcagctaaccccacatgcgctacaagatctcacttgccctatccatctacatccacccctttcctctttctttctccatcgcaccatattttttccttcacccacatggaagcacatgtctttttacctttagctatcacattaggaaggtgagtgttgtctctcctctacaaactatggcgacaagaaccacattaggaaggtgggtaacgatataaatgagcctttgaggggcaggatccaaggattaagtgctacccacatgggcactctgattccattgcctaccactagtacactgggaggaggtgaagtatggggttcatcgaatgtggtgggagagcgctagggattgcatagttgaataagggaaggattttttttcatgatacttagcattagggctataaggactaagatttcattcttaggttcgatgatgaaggaagctttgcgggttgacatgtggttggtaagacgatgacaaatttatgctttttaagactaagacctaggaatatgttccatcaagggaatcttacatctagactaggttacaagtcatcctccatttggatatgattatgtctaaaattggttgttagtttgtttttagttgtttatccatacctcatgtgcatgaacatgtatttggatgacaagtttatgtccctaggaacacgaatgatcaaacactagcaaccaacaatgatgtgtcaattttagatgtggagaaggatgatgagggcctaaatgaacatgcaaagtaccctagttcagagctagagtgtgatacaagttgaaagctttggtaatatgcatgctatgttccttgttcctttatatgcaaagataataaagataataaacttcaggagtatttatttttcatagagaatgtataagcctagttcaccaacatgcatttcatgtatataaaaatattacatgttcaaataagtaacttgtttatattacaattatttgaaaaggatattatgtttttcatgagctttagttgtccaagtactcaatcacacactcatacaaattggcagtgataactaatatatt

SEQ ID NO: 5 - DNA, 2819 bp, Zea mays
  source 1..2819: mol_type = genomic DNA
cctctacatctcccctccaattcccaaagagtcaaagacaacggacgcagctagctgtagctctctccctcctccggcgcaggtggcgcgccgtacgtggaaagatcgggccgcttcgacacgggcgcgcggagcaagcggactgaagggcggtcacttggcagcaaagccagcaactaccacgccaccattacgacgacgacgaccggcctggaggacgagatcctgcctgcgacgtcgtcgcgaggttgttgacgaagggcgtgcgcgtgcgcgagctgtacgtgtatatatgcaaccacagcaaggatcgtaccggaagcgttggacgtcggtcgcagcgtggccgtgggttaccggccaaagcgtatgtgacgcaggcacgcagcgaacgaaagcggccggccggccagattcaacgccgcgcgtgctcgcccggccggccgtctggtccaccgggtaaccacactgccgcgctgcctgctcgtggagatgcattgctcacagtagtaactctgtcgcctctgatcgagaggtgaggccgtgatgcatgctcggacttcttctctggctcagtgacgctcgcattagtggtggcctacttggctcgctctcagccagcacagcagtgacagagcaacggatcaaccgtctctgcacgccgcacgcgctactagaagaccagtcgaccacaggacccggccgtctcatttccacgcatcagtaaatcacttccagatgcacgtcgccctccgggcgccttcgcacaccgtagcccacggctttctgtcgatcgggccgggcgggtccctcggctcctttgttctttcccggcgagcggcgaccgcgccacgcgacgcgacggcgccgtgcggcgcgcgcagcgccacacgatcgaatccagtcggccgggcacgcacgcacgatcatcacgtgctcctttaatttcccggagcgcgactacgcccctgcctccatcagtccatctgctccctgcggtgaggtcagccatgcatgcagctagcgtagctaggtgccgttggatcggccgcgaccgtacgtagcactatatagcagcaggcgacggatcagaaccccctctgtgttcggatctgtgtcggaccagggagggagggagagagagatagcagtgcaggccccgccaagcgcccggccgccagtggccacgaggatatctggatcgggcgatcaatgatccggtgccgtggccatcgaccagagcatcacacgacgcgttcggcgcgggggaggcttgtcagcaggcagttcgaccgcgcgagagccccggacggacgccccgcgatacggacggagggaatcccatggcgggcgtacggccgcggcgcggcgcaagggatcgatgcacgggattggaccgcagactgacagcgagatcgcggcggacggcgggcctgccggccgacatgcaccggccagggtagtacgtacgcgtactactagagacgtaacggacgatcttgcatgcatgcacatgcaattatgcatgcacggccctaaaaacagacactcccgatactatgtatacatgctgcatgcggccaagcaacgacggtggcgtatgcatcgagggcgtacgtcccccggcccagcccggctgggtccagggtccaggcgggggcagcgtgcacgtcgcggtcgcgaccatgatgccttgccttgcctgccctctgcatgcagcagatggtgtacatgtcggcgacgggcccacggaaaaagcagcagaaaaagcgcacgcggcgaatgaagaagggaaaaggaaacgcaggtgcgagagggagcggatgcatggatcgaccgagccggccgggcgtgagatgcaatgagtataacgatgaagtgatgaacacaacacacccccacccggccgcagtgatggacgacctgtacgacgcaattcagctgttcggtgcggcgtcgccttcgggccgattcgccggacggacacagcgcgctgcatgtacccacccaaatctagctaacgaccctccgatccctctagcatgcagatgcagtgcaaagtaataagacaaaccccgggtcggtgagacgatgccacagctagctactagttccaatcaatcagctgggagagggccggttctggcccggcctcccgtgctttgctagctttagttttggcatcaacgaatcgtcgccaacagtcaccgacgacgacgaggaaggaggaggaggaggagaagattagaagggtgtcatggtatggtccacaaaggcattatattgatgcccatgcgtgcatgtaaactgtaaagtcttcttaaatcttaacggtgtgggcaatgaaagctagctagctagcctgctatctactggagcagcaggcccgtgtggacggtgggccacggacccgacggaccaggcgcggtgcaatattgcccaacgccgggcgccatggaccgacctaccaacgtcgctagctagctagctagctgctacctatccatgggcggggtttggcccggccggacagggaacacgatgagatgcatgtgtgtgtgctacgcggaaacgaagagatcgaattaagcgagatctctgtatggtgcgtgcccttggttccgcggccctgccgttgttcatgtacgtcatgttcacgaggatctcgcatcaggcctcctttgctagctgtccttggttcaatggttctggattcgtgtggccggtctgtaattcactgcactggcgagattattattgctactggatacaggcaatatgtcgagcaggccatccagtctgggttcattcttggtccc

SEQ ID NO: 6 - DNA, 2116 bp, Zea mays
  source 1..2116: mol_type = genomic DNA
acacgacccacaaaggaaaggaatctacccccaggcaaagaaggcttacggtacgggcctgttagaccaaaacccctgcagcccacttgcaaaatagcaccgaagcaccggcccatctaacgaaaccctgaggccccatccaacgaaagtagatgatgcaagttcgggtacgattcggccggcgctcctccccttgttgggcctgtgtctcccatcgcatcattcattcaccacatgcggcattttgatttcagcgcagcattatttttatacatacgttgggggaaaaaaagtgccaaaggccgaagcagtgaaacttgaaagaaatgaagagacagaacagttggtcaagtcggcagagtggcacggcacagtgttgcggaatgaaatgaagtcggagatcctatatgggcgggcggcggcgaggtgcccggtcgtcgtcggccgcgcgagcagtgtcacatggcatgtttgtgttgtgccggggcacatcagcaggcgaatcaatcaatcaatcaatcaatcagtgcgtgctctcgatgacgatggatcggcccggacctctcacgcaatgcgaggtcccgtcctgatcacatgcgctcctagctacacagggccctgccgggttcaatccacctctagctgggactgggacgggagggattaggattaggattgggattaggactaggcaacctagctaactgtggtaggatcgcagcaactaaccctccttcactagcgctggtcggtgtcgttcctgcgaagaaacgaaaccgacgacagctagctactgatgatcagagtactaccacaagggaataataatgtcgattcatatatatagtacgacgcgtcggcaatctaatcatcgacgccgaccggccggtttagcacgaggtatagtatatagcctctggctttgtctctcacacgctgctgaggtgacgccacatggcatggagatatatatatagggaacatagcaaaacagtaggcatgcatatgccaagcgcattatatttcgctaccaagtgtagttaacttgacgtgatgatctcatggccggtttagctgttacctctctcttattatattgtacgtactccgccatattgttcatggccgccggccaccaggagaatgttgtgatcatcatgcatactagagcgatgatacatctgccgctagctccttatttctttcttcatcgacaacttttttgttggaaccgaacgcgtgttggagttggaccacgcagcacagcaggggacgtttatacttgttggtaagctgttgttagcgctaatgatgacaaacagacgaggggaagttttcgcctgaaaacaagctaaaacctaggagctagctacaggtgaatcgaactagagtaggtaacttaacttgagccagtcagtcatgcgcgctgggctgtgcaatgcatatgcaccgctgacaattgtaggcccagctgacagaaaatatatagacacatttatgtgcgagtcacgtcgctagctctggatcttgagtcgctttgaaggaggcaactgatcgatgtctgactctgatgctaaccatacccttgcagtgagtgtgagtacaacgtaacctactagaatcacgcatcaaattaacactcgtcgtcgttgcctctgtaccgagtaattgttgctgctggcgggcttccagtttttttcattttctttttcagatggcggagctcacgtccgcttcccaggtccacgcacagctcacgtccggctccccaatcaaccgaaagctgccgctgagctgagactgagacggaggggctgaccggtgggggcgtgcgtgatgccctcctcctcctcggaaaaggtgagtgggtgggtggccgagggccgagggccgtgcacaggctgacacaggccagttgcttttctgagctctgcctgtctcttctgcccagatatgggacaatcctgcctggtctcaatcggggttgagaacatctctttgtcgtcaccacggttacttacacttacctcccgtggtaattaaccaggcaaaagcaattaactgaacccaacgacgtgtacgtaccgtaggacagacatgtcgggagcccggacac

SEQ ID NO: 7 - DNA, 1634 bp, Zea mays
  source 1..1634: mol_type = genomic DNA
gacaaagtacacaatcaacccctttgcaaaattgtgagccattcatgcataaaagaggaaatattgctctcatatctggtatctatcttttccctccaattacagttaaaaaggaaaaaggaaacgaaacgaagaggaaagtaatattgactgttacagatcttattctccccaatgcaagtggaactactttgttttttcgggtcttcgtccatgcttttttttccctccaaccatctgattgttagatactgtcatttcgccattcaatcggaatcaatcttcaagttgccggagcaaacggcccattggaatcgaacgagaaccttcggggagagagaggagctggggaaaatgttctagctagctagggttttgtgtgggcggacggtgactcgcgcttgcatgcattgcacactctccggccggccccgaccctctccgtggagccctagctagatcaagctatcgcactccggccgtcccgtgaaaaagaaaaaacggtgtttggagaacacgcagcacgtacgcatcgagagtcccaacacagtgacaggaccacacccccagatcatgtctcaactctcaagtagatggccacaggcatgtgatgagtctgttgcagttgaacgatcatgtcgtagtcgtcgaccgcaccggttttaatttcctgatgacgagctagctagctctctgttttcagaatcaattatttagacatgcgtgtgctcgatctacatatatagctagctggccgaaaaggatgaaggtttttcagccatgcatatatatgcatcagtgcctgtcgtctccttcgccatgcatgcatgcacaccaatgaaacgcgaacaatcatttaggacgacgacgacgaggaggaggactgcatgctgctagctgcctgcctccctgccccgaaaacgggtgcatgcctgcatgcatttgcacccacacccactgcgcaggccgcagggtcgggcacaaggtcaccagcgccggagatggtccaagcatgcacagcacggctatgtatcgagctgcatgcatgccagcagctgttggttggccaccgggatttggtctcctcccgcgtggccatggcatgtggtggtctgatgtgaacagctgctgctgctggcttgttcgtagcctattccggctgcatccactacggaccgacgatacgcggtccatccggccagctacatgcagagacgacagatcatactagctagctagctaggggcgccgtcgagaccataccggcctgtgtcgtgtggcctgtgtggggaagctgaaggatgcttcgcagccacatgccgccattgcttggccgcgataagcaagccatccattcgcggccctgtgtgttctgcgtgtagctctcagctccgccgcacaagcggagaaaaacaaaagatgtggccacgattttgggggatcattttcgtggccaggcacgcctccgctgatgtggataggtatccaagcaagtgaacaaagtagtggtgggcactgcaccaggaccaggggggaaggaaggaagaaggatgcatgcttctattcaatctttcacccatttgccaatgcgcacgtataactcgcccccgcgttgcctagctccctcgacgacg

SEQ ID NO: 8 - DNA, 4840 bp, Zea mays
  source 1..4840: mol_type = genomic DNA
cggttgtacttgtaaatctagctgcttgtacgatagttatatgatgtgtttttttatccatagcgaccaccacgacaatctagacctagctagtgcgactgatttttacatcaatacatcgcaaaagctacgcacttagagagaaaaataaacattggaatttagagggtacagtatttagcacttcgttacagatgtgtgaaaatccggagccgattttgcaatagccgtggatcgctgaaatcagaccttccttgtgctgtctaaaataaataaatgttttgtgttcttcgtcggaacaagaacaagcaccggttgctcggaccgtggagaaaactggttgggctgtgccgagaaacaatgagcgggctgggctgcgtcttgacggcgggccaccaaactgtcccgccgtgcgcgcagggcaacaaccacaacgtcatgtgcggcttgcttaactgggcccgttagacgcgggcttcgtgtggccttggaacacggccgttcgacctggcttcacgtgacgtgaaccagagcggagcggggccatcgatttcggccgcgcgaaacgcgtgcgcgaggcctgcgaaaggccgctggatgaagctccctttgattgaagcccgtgtgggccgcaccgcatggtccggccggcgatcgtgaccgttggagtacgatttattcgatgcgtatgtactcagctcgatccatatacgatatgatagtacgtagacatcttagacgtaagttgtttaaggaactctctctctctctctctctctctctcgggtttctgtgttcatctcaaagttttttcagttcaaaaaccaattcgaaaacaaatcggcttaaaattcaggtaatcaggtcaagcgactttactctggtctgaataacttgagacatccgggttgccatggccgactctagacagcggccataaacacggtggtttctttttcttattgggatagtaggtcactccaaataaaggctattgccatatgctaaggagacggaatttgtgacgccatcgccaccgggttaacgttaatattctactactagagaatctagcttacgtttcggttccggccggccagtagaaaactctctctgaaccgaccggtcagaatcccctgctcggtgctcggttgcttggaccgcacgcacgcacccctatatcgtcagtgcctgtaacagttcttattcggtgattattattataatattattccacgtttgcacacaccgcacatccgcccgtttgtaaaactgtgagttgatcgtcgagacgaaaggtggagctggagtatatggctgggcttcgattgccgctcgcaggtcggtgtcggtcccgaacttttttactcgcgctccatcaccacatagcctggcgatcctatcgtctgcctacggggccgcagcggcgctcctccatctcctgggtctcgttgtagccacatatagagtagtagattgttcgtcctcgcaatgatccgtagtgcacaatgcccagtcgaatagtcgatgaatagcacatacacatatatgcgtgtgtgtggtcttgtcaaggttaactgctgcagagatgagatgccaaagaaaaaacacatattctaattaataaagctttgtgtgccgcgacaagctagctaggctactgtctcgtacgttcacgcggtctaaatcacgggcgcagcacaaattcgatggcagcctggactaaacgaggccgtggccgtcgtcaccattcaccgatccacaggattcacccgggggcaaaaccagcgcacattacctttgcaggacaggagttagaggcgcctttttcctggtccctctctctgctgagcacatgcagcagctagctagctcacgctactagtcactcgcgaagaacgaatccccggccggcgccactagttgtggctagctctcgcgtctttacattcgcagctgcagcgtccatttcacaggcagtatacatgcatgtgatcgagtggaaggaggagaggccaccgctggccgctgcccgctgcttttcacgtacaggcgccggcagtgcaatttggcgacgatgcgaggtgttcgccagtatgtctcgctgaaagggtgaagccggagaaagggggaagaatgtttgctgcggcggatggagataagatcgcatctcgatgggaattagaacggccgccggccgagtgtgtgtgtgtggactgtggatcgaacattccgccgcgcgcctgaaaggactgtcgaagcattgtgacatctgtcagtcgatcgatcgtgtggttaacttaacggatgctaaccctagcttcttttttctcttcagtctagctagctttctatcttgggagacagggacagcatttttctttttgtttttttagtggtacctttaattttgctggtgtcggttgtacttgtaaatctagctgcttgtacgatagttatatgatgtgtttttttatccatagcgaccaccacgacaatctagacctagctagtgcgactgatttttacatcaatacatcgcaaaagctacgcacttagagagaaaaataaacattggaatttagagggtacagtatttagcacttcgttacagatgtgtgaaaatccggagccgattttgcaatagccgtggatcgctgaaatcagaccttccttgtgctgtctaaaataaataaatgttttgtgttcttcgtcggaacaagaacaagcaccggttgctcggaccgtggagaaaactggttgggctgtgccgagaaacaatgagcgggctgggctgcgtcttgacggcgggccaccaaactgtcccgccgtgcgcgcagggcaacaaccacaacgtcatgtgcggcttgcttaactgggcccgttagacgcgggcttcgtgtggccttggaacacggccgttcgacctggcttcacgtgacgtgaaccagagcggagcggggccatcgatttcggccgcgcgaaacgcgtgcgcgaggcctgcgaaaggccgctggatgaagctccctttgattgaagcccgtgtgggccgcaccgcatggtccggccggcgatcgtgaccgttggagtacgatttattcgatgcgtatgtactcagctcgatccatatacgatatgatagtacgtagacatcttagacgtaagttgtttaaggaactctctctctctctctctctctctctcgggtttctgtgttcatctcaaagttttttcagttcaaaaaccaattcgaaaacaaatcggcttaaaattcaggtaatcaggtcaagcgactttactctggtctgaataacttgagacatccgggttgccatggccgactctagacagcggccataaacacggtggtttctttttcttattgggatagtaggtcactccaaataaaggctattgccatatgctaaggagacggaatttgtgacgccatcgccaccgggttaacgttaatattctactactagagaatctagcttacgtttcggttccggccggccagtagaaaactctctctgaaccgaccggtcagaatcccctgctcggtgctcggttgcttggaccgcacgcacgcacccctatatcgtcagtgcctgtaacagttcttattcggtgattattattataatattattccacgtttgcacacaccgcacatccgcccgtttgtaaaactgtgagttgatcgtcgagacgaaaggtggagctggagtatatggctgggcttcgattgccgctcgcaggtcggtgtcggtcccgaacttttttactcgcgctccatcaccacatagcctggcgatcctatcgtctgcctacggggccgcagcggcgctcctccatctcctgggtctcgttgtagccacatatagagtagtagattgttcgtcctcgcaatgatccgtagtgcacaatgcccagtcgaatagtcgatgaatagcacatacacatatatgcgtgtgtgtggtcttgtcaaggttaactgctgcagagatgagatgccaaagaaaaaacacatattctaattaataaagctttgtgtgccgcgacaagctagctaggctactgtctcgtacgttcacgcggtctaaatcacgggcgcagcacaaattcgatggcagcctggactaaacgaggccgtggccgtcgtcaccattcaccgatccacaggattcacccgggggcaaaaccagcgcacattacctttgcaggacaggagttagaggcgcctttttcctggtccctctctctgctgagcacatgcagcagctagctagctcacgctactagtcactcgcgaagaacgaatccccggccggcgccactagttgtggctagctctcgcgtctttacattcgcagctgcagcgtccatttcacaggcagtatacatgcatgtgatcgagtggaaggaggagaggccaccgctggccgctgcccgctgcttttcacgtacaggcgccggcagtgcaatttggcgacgatgcgaggtgttcgccagtatgtctcgctgaaagggtgaagccggagaaagggggaagaatgtttgctgcggcggatggagataagatcgcatctcgatgggaattagaacggccgccggccgagtgtgtgtgtgtggactgtggatcgaacattccgccgcgcgcctgaaaggactgtcgaagcattgtgacatctgtcagtcgatcgatcgtgtggttaacttaacggatgctaaccctagcttcttttttctcttcagtctagctagctttctatcttgggagacagggacagcatttttctttttgtttttttagtggtacctttaattttgctggtgt

SEQ ID NO: 9 - DNA, 1726 bp, Zea mays
  source 1..1726: mol_type = genomic DNA
gcggctagtggatcgatcacacatgtttcgaactatcttcttttccctgtatgaggtacagtagtagcttacggacgaagggatagatagatatatacataccatgtgctatgcgcgtctcacttgtgtacctacagctacagatgtgcatctctatcctatctcttcactctggccaccttttcttctagctcggaaggaaaaaaaaagcatgtattattgcatcactttttttttgcaagggatacggtgcagcagtactactactgcgacgcgaatgttcattcacgccgcgtacgcgataggcaccgctgcatgtacccaccggcacagtactaacggtttagatgtctactacttattaattcaatcacgcgtctgcgagaaagcaagccgacgggcatcttctgcccgagtctctccgcgtttctgtaactagaattgtcatagtcagggttgccaaacatcagcatcccgaggcagtttctttaattctgcttttttttatatatgtaagtttgcttaccgaatgagctagttctaaacaaactcaaaaacaaaacagggcaactgggggtcccttgacattgcacagatggacctgaccactttgagattcccccggcttctatctccttttccctccccttggatcaaatgaacaaaggagcgcattctctctctctctctctctctaaaagattaaaaaaaagcctgcatgtagtgttctttgacaaggacaaggaagcccttttacatcaatacatcattcgtatgttgttgttttctgtgttctttgcgttcctttttttcccctccctccgccttttttctacttgattgttgccaagatctggagcacctgctctgatctgattgtgtgcgctggtttactgaacctttcggagggctatacgcttcgtacggggacataccaatttcaaagaattcagtcatcaggtaggtggttcaatcataccgatggtttcctcactgcatcactcaccttctcatttttacgcatcataattttttgttcccttctccttaattcccatgcggtgaaggagagatgtgaactaacagtttggcgctgcactgttcgaccggctaaacacggggccaatgctctctgtacgtgcagatggataggatagtctttgattcttgtttcaagatgacgtggatagtctataatagctaaatgtttgcctcgactactaacttgccgatatgggcgagggtaactttaaattaaatttttaaagcatttgacttgttaaaaaaaataaaagcctatattctttgttgatggagggagcaagtggctgaaaagccgttgccatttctgggcgctccctaaactacgcggcaagcaggctattgggagcccttgtcgctgtcgacgcgatgtgcggcctctttcttcgtcctcttggttaggtcttatctacatggtcacgcatccagtttattcattaggtacgctaactgtgtgatcgtatgttcagtttaatttatatgtgttagagtataaaaaaatttatgtaaattttacattagcttgagtcagtcagtcagagcaaattaatttagcggctagaccgctagaggctagtcgcgtgcgtgcgtcgctgatactcaccgtcagtccgtcaccgacacttggcctgggcggcgtgtagcagcagcacggatacgggcaatacggccgggtgcatggtct

SEQ ID NO: 10 - DNA, 1923 bp, Zea mays
  source 1..1923: mol_type = genomic DNA
cggggccataggaggttactaggtttcatggccacgggagctatggttccgccatcacaggccccaagacaagtattccagatcaaaacatataaatatgttttgatttgacggcagccaaaagtactgcgacgacgtacgtacgtacactaaaccgatgtaaaaccgtactacgcgctcatgttcggcggccggatccacgcagaagtttgacgagcgaattaacagaggcagagctccggatgatcagtctcacttacctcccagttctcaacagcctcgtctgtagccaagtcagaatatatatgagtgtgtgccactgccgacacatgcatatgagctattgagctcgacaggggaaattaaagagagagaaaatgctacagtagtggaaactgctcctgatgagaccggccggccggaccagatcgaccttgcaagagcggagaggacggccggccggtcgcagctagcgctggatctcgggaggacaacgggcactgtggcgtgcacatgcatgtgcgatcgaaagaagcttgcgatggtgtaaatagtgaggtcccagctgttcacagtgcattaatttgtgcggccggcgagcgacgacgagcggtggcggtgcagagaaccgacgacgagcggaacagtttggtcagccaactctttggacctgcatgctatgctaatgtaatgaatcgaactgatgagtgcgcggcggcacctgtgcagacgagtactactactctacagtggttcatggtcaccgtgctgctgtgtgcctgtgcaccgcgaccgacgacgacgccgcgattcgctcgtgcacgcgagagccagtcagccactgccgcccttgcgcatgatgcggtcggtgaacctagctagctagctagtcggcgcctgccctatgcatgcggatgcaggacacccacacatgtgacagcggcatccggcagcgctgctccatccacaacctgcagctgccgatctggcatcattaccaagagagagagagagctggggatggattatggatatgccaaagcttctagcgcaacgaaccgacacagacacagtgctctcgtacggttggtgagacggctggctggcatggcatggcatggcacaccccatgcatgtggacagtggactggggggagaggagaggaggttaccgatggatgtcggttttgcagaatcatcctctactaatatagtaactagggatccgcacccgcgccctctattacctgaggcacgacgctgcgtggatccgatcatgtgggccgcagccagctctggcatttggatcgcagcacgatctttaccgagcaaaaacaaaattattacagccgcgactgcgatgtactactagtactcaacggcaccacggccgttctcgctcactcgtcgcccacgccgaggccaccaccgtgtagcatagcaatcagtgacgagctgacaattggggccgtgctgtcacgtgggccacttctctccgattctctaccactcttttgtttccgcatggtctaagtggaccccatcgcctggcgattctttttttccttctttactttataataattccctcgtcccgtggtcgcgaggatagacagacggatggaggaagacggcgacgggagatgcagatgcgtcacgcgtgtgctgtgccgtgcggtgcctttcggggaatttgtgccgcgccaaaggggagcctcgtggggccccgaggcctgccgcggacccaccggggctcaccaaaagctggctggatattgcttggcaaaaaggacaccaaaagaaaacgggaaagggttggccgtaaataattggcacacgcagcatatgcacacagcttcttcaaaagaacaatataagtaggcgttagagatccatacgactatacctattgctgtccatatt

SEQ ID NO: 11 - DNA, 1660 bp, Zea mays
  source 1..1660: mol_type = genomic DNA
ttagaatttgctatgcaataaatatttaaaaaggtgcatataagagagaaaaacatggatatagtgacgagattactcttgttaggtgttccttgaacatctaccctagtcaaagtaaaatcccttaccacccttcttatgggatcacacaaatgatgggaatttatacccaaaaattccatgaaatatgcaatagaataaaagaacgtggcacaaatttgacatgtttgagatataaagttatcatcaatctatagtagccaaaacaaggacaaacaaaaaagtttaaggaattaattaggactcaaacatataggaattgaaacacagacatgacaatagcaaatagaaatcaacaattatgtaagtttttaacacataaaaatcaaacaatggttatagacttgtagttctccaatctctagtttttcattgattagcaatgcaaaggttgtatgggacaaaaactcaacataatccataggcatcaagtttactatggtatataagaccagcaaaactaagataatattatggtcgcatatcactcattataaacaagggtacataaaagagtacattctaaacgtttttacaatattgcatgcatttgcttattgcactagtagggacaaaaaagacttctagtcagagaggagccaacaacaatgccctttgaggtggttaaaacctagagggtgaagaagggcttctttggccatcaatgaagcattataaaatcatggaaccacttgttactcaatctctcactatttgtctattcgatgtgtgatgaagtgttgatgatacattgtgtgttggcacatgggcttgtgttctactcattacgtaggccatgtgataggatgagtagtggctaactcgacaagtggtgaccaagtgaggtagggttacatagtttttatatatatgatattatttgggcatacattatacctatattattgggattattttggtcagaatttgaaatgcattctataatgttcgaaggccctaacgggtaaccaagaagggtacataggaaacatgttgattgtaatgtattcgatatccttctaaaatttggccataaagccctttatagggagtgaaaacaacacatcaatgtctcgtaacggagaaatccctattatttataagggattgggccccatttccatgcctacggaatgcttatattacaagtgcactctagaaacgtaacttgcacatggatgacatgatcaagactcgtcgcatgtgatacaattaccttttctactccacattcttatttgacttttgtctctagtattttttgtttgtcctagacaccccatcagtagagtccacctccttgtcaatgaaccttaactacccaccaccaaaaaatccctctttctactttcattatattggtataattgctatagctatcttgttagttgcaaaaagactagtcccattgccttactagtgaccctaatggagggctacatatccttggtagatgtggaggtaccaatggttccatcacatccattagattaggaggacaccatgataggcactagtctcaacattaaccatagttcttgttttttcttttaaaatcgaaagcattattttgttttaaattcttttagtcgaaataaac

SEQ ID NO: 12 - DNA, 1552 bp, Zea mays
  source 1..1552: mol_type = genomic DNA
atatatatataggacagcggaagtagtgccgtacgtgtgcaaaaagactccctacgctagctagctagctagctagctatatatatgcgtgcattttgtagggatcggagagcttgtcattgcgtggtatatgttcgttcacgcagtggccctggccgtgtatatatagctagagctagtgacctttcagtgtttcatctagtcacccctatcgtctatcctcattggagcctagcgcgcgcgggcgcggtgacaggaggccgatccggcggtcgacgacgggcgagagggacgagatcggatccaagacacacaaaaaaaaggtaggcagcgaccgaccctagctatgtatcctacgacgtgcgtgcatgcaatggtgatccatcctgcagtacgcttgcgtgcagtgtgtcgcctcgcctgatctatagccaccacacgcacacacacatcgatctctacgaccgagctgacgctgtcgtcgtcgcttactaccacttgacttactacactgccgtaggaaaggcctgcgccgtacgtacatgctcacgctgccctgctggtgcctggtggtggtggtggtgtggtgtgtgtcgatcgtgtgtgcatgcatgctggcatgctgccgtcccagcgcccggccggtcacgcgcgccaacagccaacgctaagtatgtagtggccgtggtcgatgctgtggtaaacaagccatgcactaccaccaccagctagcgtcatgtatctcatatatatctcaactgagcatgatgcgtgtatacagtatataagcaggctagctgcacctgcactcggaagaacaactgcatgtgcgtacgtactagcagatagaatagctagcagctagctagaaccgtgtgacgtgttgtgatttgaccgatggcaaattaaaaggtgctctaatccgttatctgtccatatgtatatgtggaatccgacgacacacacggcgatcgagagtgatccatgagtggccgacgtgtattacacacatcttatatattagcttattaactgtgtcgtcttgtgatgtcagctactaggtgccgccgtgccgcagtatgtagctagctcgtcaccaaccctcaccccggcccctatatatatctttgaattatatatgtatgtatgtatagtatgttttctcaagctcaaaatatatatagttgatgttatacgattcaaatattgtctcaaagcagttattactttggctcaaatatttttctctacctctctttttctcgttgttttacgttgcacatgttttttagaataaaagaagtatatatagctttacaagtaagtatgtttaaccaaaagaagaaaaagctaagcgcatatgtcgtcgttgaccgcaataaccagcacggaaaattctaacaaaccgcccgacagggtcaggacgtcaggtcacatcgacgatctgctggttggaagaaacatatggacatggagcccgtacgggccggggggagaaatcagatacgccacgcagacgcaccaccattcatgcgtttgtatatgctagcta

SEQ ID NO: 13 - DNA, 1636 bp, Zea mays
  source 1..1636: mol_type = genomic DNA
gcggtagtagtagccggtgtttggttgctagccacacacttttgtgtaaagtaagtcgatcaagttggtgagtacttggttggttggatattaaacttgggtaatagtctttatagctcgctagctagctccaagtatatcatagagtaaaaagcgtgacaaaattgagtgattcatcccttgagcttgcattgcttgctcaattgactactcaccagagctctcaggcatctctctagggatgatgttgcgcatcccctccgtaaatgacggcgccccctcccgagcaggcctgccttcagtggcggctaccttcttgttggaggtctcgtctctctctctcgagcggtatgcagtatgctccccgtcgactctgctgtctgctcgcagtccattcgtgcaggcagaaccctaccgctatactagaagtaggtagtagccccgcctccgacatggaccggaagacccgctcccatgcactcctcaccgtgtcccgtacctagaagacgggatggaggagaaagacgtgaaggagagacgagtgtgtaacactttttttttggggggtgggcacattaatgtgtgtacatgagcgaccgagaccagtactacaagtctctcgttcgtcttctctccatcgatcgaaacatgccgcaccgtcttcagtagttctctctgctggcgaaagggcatgcatgcatgtgcggaaactatactagaacattaacaaactaacaaatataatcggtggtctgtgtataatctaaccgtagctatatagcaagcagtgtgggtgtgggcagctgctggtgtgcggcgtgagaaatataggagggggacggatcggaccagaacacaacagtcgaagaacacggggaacagtgcgcgcggtgggtggcgcggcaacagtagtgctagtgccggccccggtccccatccgatgtgaccgaccgctactactaccagcttgcattgcgtcgacgacgcattgcttgggccgcgcgcgcgtgtatgtgtgtccctgtcacctgtcgccaccagatcggctcgccggccgcccgctaccgagatgaaagtacagtacgttggacgagcgcgcacgctggctttgcccacgaccacgagcagtgcagtgtgcaaacctatctatccaagcacagtatagctactacatatacgatcgatctagctagctagggcgactgtagccccaatgtacgtacaatatacgtacgtacgtgcacgctgcttcgtgtcaaaaaaaaggccactgtacctacctcacgaagtactataaggacctgtcatgtccttatatgagacatcatagtaccgtaccaaagtatgtacacgcggtgcaccggatcgtctcaggtctctatcaagcgaaggccgagattggtgtggataccaaatcgaaaacatgcagtacatttgtcgtcagcaggctatgcccagcagcacactcatcctacgcatcgatctgcgtctaactattcaccttccagccccaccgtggatgacattgtgcaaaatgagcgctcgacgtacgtaccccatttctttggctttctctctgtctcctacgactgtgtatgcgtatctgctgagatagagcggtcactttcctcgggct

SEQ ID NO: 14 - DNA, 1584 bp, Zea mays
  source 1..1584: mol_type = genomic DNA
ggtactgattacctaggaaaataatcttgttgagggtctagctggtaacctggttgataagggggtggctattctactctattctgatgacaaatttgtgaattaaagatgacttatacattgctagaaacatgaatcttgttgtatatttaaataatgctatttggaccaaagattaacttcactaatattgaggacccgatgattaatgataataatgaaaatttaaacacatgctgatgtttaattgtcaagtgagtatgttccccacaaagtatcttggggtccccaacagactctacatcattgactggctacacgtctatgagaaatctaccaaaaaattagatgcattggcatgctagttccctaacaatggtggaagaggtgttttgattaaatccaatttgaataatcctgttatatatcaaatgtctatatttatcttactccaaaccatcattgataagattgataaatagagaagatcattgctttagtaatgagggaggtttactagcttataatagaaaatgttatctattcaaatggactaagtagggaaaagtgccaagaaaggaggtttaagaattataaatttcaagcaaatagatattttcttagtaaataattggtggtataaattagtaaatatgtaggggcattaaacaggaaagtactcatcctaagtatattagaactttgtgaatttggaaaatcaagcatatacatgatgattctcgaatatggacagatttactaattttaaattaggcatggaaaattcaagtcaataatggaaagggaccatattttagcttgatgcttagcaaggaaaatttcccctccatcgtcaacaccctgttctatttgtactttgtaatgaaaaaaccagtgtttaggtttaaacatgtaaatggtactcaaggaaaaaggtagtgacatccaagctgggctcccatccccttgctatgtgatttttttgagttaataacttatatgcatgcaaggattgcaatcatatatttcattctccatattagttgcatatttctagtcatagcaattgtgaaggtttcttaatgtactttgactatgtgctcatgtaaatttactccctacttcattcacccttgggcactgatgtaccctaaacttgatatgatggtccaaacttctctttgggagaaatggaatgaagttcaatatcattccctatagagcattgccttcgccatgcttaggccatggatcctcctctagtggccctctaggatgaaattcatgttcaataataggtcttactatggtctataattgatggcatagtcgccttcgactccaagctccactatgtgccaacatcacccttcctacacaagcagatgttcatcatctatgatgatatacattagggcatacaatgcatgctttacaggcttcgctaagacttccacatgcctaaccttcataatgtggtccatgagtttatttgcaattgctccatgttaaatcaatacaataccaaacacattcgcgtacatggggatcttaatgtcattgcgtatttcgatggtggtttga

SEQ ID NO: 15 - DNA, 1776 bp, Zea mays
  source 1..1776: mol_type = genomic DNA
tgcggctgatggtgtaaggtgctaaacagtaaacactgtagctgcaacgctgctagaacaggcagtggagcttggatcaccttgctgtcattttggttacgacttaagcgattgtctattttgtcaacatgctttaatcttatatgctcttttccttggaaatttgcagcttgtatctgacattcatgtagccccagttggcactgttctcgctcagggcatgaactcaaagaagaagcacgaagtaggtaattcggcttcatactgattgttgaactttctatgtcctcttttttttcctgcaaatatgttgttatttgaactctcatgtttgaagaaattgctttattcagattgaaaatctagctgctgtggttcatgcaattgcaaagaggtgtggagccaagacagtggttgatgtaggttctggccaggtatgtcatatgcatgctagcatctgtgcaattgtaaccagattcctagtagtcataattttctttctaatacttggacagttggcatccctctttattccttttggttgtgattatgccatcttaaatgttaggtttatgcattcatcaccaatcccgagcaagaatgccccaatcgatccagctaaatatactgcccccgaggaaaagcaatatactgtgctttatacatgtccatatttcataaactatgcctttatcgcatgatactcttgaagtctggatgctaagagtccaaattatggactgttttactcatctttaaatagtcagaacctacaaagaacatgttctacatggtctggtctgttattttgttgacttaattgcaagctgcaaacaaatgaacaagtcaagctatctggtttttgttgtagacattgcatggcaatgccttattttttatgttgctgttaaatgaaagaaatgattgaatttaacctagtatcagcagtaacgtgttgtgtcatgcatttatcttatttcccatcttatttgattatcagggttatctcgcacaggccttatcttttgagtaccaactccgtgttgtagcaatagatgcttcatcacaccatgcatcagttacaattgctcgtgcagaaagaataaagaagcactatgctgctaaatggtgcaaccttcatcaagtttaatttgagttctactacctttggattcctatctaatggtataaattgtgcctattatttttgttagtgtggagaagcaactgctcatggtacctagggcagtcacctgtcatgttctttctagtgacacattggcagcagtcacattagatgcatgtaaggatgacaatggagaacatgtgagagatactaaaacatctactaagaaaatcactcaaatccaggaatcaactcagggcacccctccattaatcctggctggtcttcatgcatgtggtgatctttcagttaacatgctaaggttggtatttgcgaagttgagaacatggccatggagcatggtcctttctccaagctcgctaaatgttgcatattgaatataagtattttccaccttggtgaaagtccttcaaatattacattcttttttacagagtttttgtgtcctgtgaacaagtaaaagcattggtaagcattggctgctgttacaacttgctttctgaggatacttatgaggacacagatacctgccctggttttcctatgagcaaggctgccaaacattctgaattggtacttgggaaaagcatccgtgaccttgcatgtcaggtatgatagcagttcatgat

SEQ ID NO: 16 - DNA, 2025 bp, Zea mays
  source 1..2025: mol_type = genomic DNA
ccatggcattgtcatcacgatttgattaacttattcctactctactaagacaaaatctcttgaaattctttaatcctacccctcaatgctcacctgcagtgaccccacctcccacgcccgtggtcatttccacacgtgactcccctcccttacctctcgacctccacccttgttctcccttcacagactccattgtcgccaccggccttcaccacctagaagtggcaatgcaaagtgcggcctcacctacggttgtcgaggttcgggtgaatgagcaccttccacaacgtagatggtgcaaggaaggcgtgttgttgggcatgcaagcggtggtggcatgtggcctctcaacgctacgacacaggaggtgagaacgtcggaatcaccggcctcaacccttaaaggcatgtgtctaagatgaagcacatggatgagcttagcatggcccactaggctagctatgttgtgtcggtccgatacgtaacggattgtgccttagtgggcctaggcccgtgctaggcttggcgccgtttggtcatccatatatagtcacaaccaaaggaacatacgaaagatacctatttttttttcttttgttttataatcactaaatgtgtaacaaagatagaggtggcattatgtgaacttgtgagatgaaaactactgccactttctactagcatggaaatgcatccaaatgctctccatgcatgtgagatgccaaattggcaatgcagttgaacctatcattgttagaattagattctccttgagtgaggataggggggaaaaagggcgtcctaatcgcttgtgattccgatgaatatataaagtcgctcggtcgtcatgcaaagtgcgcaaggaatgctctttaaggtacattactctaccctcctttatctttttttttcctatcagctgaatgtcctctggatcggcaagaatgttcggtcttgatgctagactagaaggctggctggatcgccctgcaatctttctctctcgaggattgcatgatccgagcttctctggcgctcctttttataattcgacacgaatttggcttgcaatcatttctgcgttattcttgctttcggttattttttttgtttctcctcatctgatactttgcatatccacatcactttattctgcggtttgctttattcttataggctaaaatctcatttcagtggccttttggtgccatcctttttggtcccttgctgattcgtttcttagtgtcttggaattgtagctgatcgatgctggtatcctgatgaccaagaatatatatatgacactgataaatgattgggaaaaaaaaggggtccatggaggaggtcagtatgataaaaaaaatagtgctcagaagaatatatatatgttagaagaaaagttgccatcgttcccaaagttcattaatttttttgttaccgtcaataatcttattattaagagtaagaagtatattattggctgtatgaaagttatactgctagattttgtttttgcctagaaatgctttaacagaagtaacttttttttcaccaaattacatctgcaacgacatcgttaaaaaatgagaagaattaagaatagaaagatgtttgaaaatcactccaggtcacctcggacaaaaaacctgcgcctttggatgcatagacggctctggtcccgctggaccacctgatcaggaggtgtgccccgcttgagagagatcagagatgcggcaaagagtgcatgcatgcacgcaccatttgactctctctcccctctctctctcttttcattgtaattccaatactactatggagtactatttattattatgtgtctatgttactgcctgactgaattttcacactgtagcagttccaagattggagcctcaaaagatgagcttcttcctccaacattatcgccaacgccaatcatgtgtgcgtcctacatatgtcgagtgttttgcccagcgcgaccaggatttctgtagccatgcagggattgacctagggaagaaagtga

SEQ ID NO: 17 - DNA, 2103 bp, Zea mays
  source 1..2103: mol_type = genomic DNA
agaatgagagtacactcatcatgcatgcgctttcatacgtaacagtacattggatgctcaaacgctctacacgtgtgggccggtgcaccgggtacgtactcggtgcagtgcacatatatatacgtgtgtgtcggggccagggccgggcccggggccttggttgctgtctgtagtagttcatgtaggtgtgtgtgttcttgtgtggggctggcatgcaaatggtttggtgcccgacggtcgaggcgatccatgtccatatggccatgatatacagtacagtatggctgtaaagccgaagacaaaagcctcgcatcgggcagaggacgacaaagagctggcctgtacctgtactgtacgtacttgctggccggccgggccgaaaccacgtccatccaccatctccattccccccgtcgctcctgcggcctgccgcctgcctgccacgctgccaggtccagctgccagcgccagcggccagctcaccaccttcttgcccctcacttttcagttttcaccattcaccggcggactcccttggccctcgggctgcctgcctgtctagtgtctactacaggagtgactagcagtgcgagaggccgagagcagtgctcgtgctctgagcaccacgtacacgtacagtagtggcacgtgccaatgccagctgccatcgatcttgagccgtgtgttttccagcaaggattccggcaggctccaggcgagaccagagtaccagaccaccacatgcgatgcaagccggcttgggagtgcgcgcccgcgtagacacggaaggggggaacggggatcccagttcgaggttcctgtctccccggccccgcaagcccaacaaatggccgaatacataccatgcgccacgtggagcggtggtgcggcacagctggatccctggcgacgagacgaagacgagaccgtccgtcttgaccgcctactgtactgtacttgggctgcgtgcgagcttgcaggctgcacgcagctgccgcctgcctgcctgtctttctttctgtactctgcagctctgcctcaaccgctcgaccgaacgccacagtgacactggcagcacggacgcgccaggacgaacggacccaacacagtgtgccagtgtggtctggccgtacgtttgtttgttactgttgcgatcccgatcgtccacggcaacgacgacacgccgacgactttcggccatccatccgatccgacccctgcctctgcgagctgtaattaaactcctcggcggtagtacggtgcaccgcccgccgccccgaccccaattcccgcacgcggccgacggtttccgggcagggcagaacaggagccgccgcatgcaccatccatcctagccgtagcccgccgctctcggctccccctcccaacgacctgccgctgttcctcctctggctgcctggtgcctgcccgccagcgccaggagacgagaccacgctcctccaccaccccttccacgtgctccctcctttccttgcctttcctctcctctcctctgcgtggactggacactgggctgccctgcgcaactccctcctggacgtaccttaaaaccctacccttttcccctgctgctcgtgttgaccactaccgcctgttcccagcaggtcactccctcagaccctcactcagacactcatctttctttgcctgatccggccgttacctcttgctacttcctttccctttttttcggccccaatgcgaacgaacggctcccattcccttcttctttttgtagacagacagacagagagagagagagttttcgcgccgttgcgtgacggctgacggggtgtgaggcgccgattgttctttcgcgcggtaggattacacgcgccaatcaatcatccgcgtgtgcaatactatactgctactaccagtaataaacaggcaggaaaagccaacgcacctgcctgcctggcctgctggctgctgctcaaatttctgccggcgccgcttcggcgtggatgaggatgaggaggagggaggaaggactactggagtaggtagtgggaagctgacgtggcggggtggtgcagcgccggaagcggcatctcgaggggcgcgtcttcgatcgctagt

SEQ ID NO: 18 - DNA, 1535 bp, Zea mays
  source 1..1535: mol_type = genomic DNA
gtgggtgtgggtgctttgactcgcggtcctcgtcgtggtgcccggtctactactaggcttgggccaccacgccagtgctggattctctgcctcgcccactccttttttgcctgcgattttcacagctgacagctctagacactagtgctagtttagggtttcatttgttcgtgtcccgctcgcctcctactgtcaaccaacctcttttgctcttcgcctagctagtcgtcctcctgtgaggtcaacttcggggaattttccacggtcgcgtcgcgctctcgccctttctgttcgcttgctgcacaacttcggggtacttcttggctggccagttaatacactttcacccattagaggccattaccagagataagtattccttgcttggcgccttctccgtcggagatgccttaagactaggctgctgggactctttcggctgttcaggaaaccgtgggagagatcgctcttgcacttgcgccccaggcgtagaatgtgttcatagcgagctcccatgatggatcctcctccccatcccagctaagctactagttcgttcgttgcttccttcttcctcacagcagatcgaggaaggagacggacaagaacaacctaatctggcctagccgtggatatgatgcagcgtgcaacgaaatggtcaaacgggacactagcgcacggcggggaaacgagacaacgtagcacggcaagctagcagcgcgacgacaaagccatcgccgtcggcgttcccgtacgttcccgtgctgcaaattgcccaccgtagcggccggaggtgtgcgagttatccaagcacaagcagcgtggtcaaggaggctgcctacttggctcgagtggagtggaagtcttgcacgggccagccaggtcaccggagactggctacacgccgcctgcccgctgagctcgacggacggcgcctcaattgcctcttgcacttgcactcccgctctaggtggcgaaacccatgacagcggatgctaatactacgccgcggaagacggcaggtggccataggagagcgccgcagacaccaggacagccggcgaggcgcagcgggcacagcccagcaactaccccgcaaggctcgtcgaccgctgcatctgcatcgccaagaaagtaaacataaatgtgataactatattttttttaatatttatatatatattttttattttgtttatgtaaataaaaaaacgaaaacataatatagtatgggcccagcccaccgtgcctatttccccttcatggacatactttcatggacataggagcgtggattcggtccacgggatcttggatttggtccgaaaggtcaccgtataaaaaaataactttttaccgtacttaacttttgtatattgatttaattgataattgaatatattatatatgtacctattatttaatacattgcatgtattgaatattgctcctagtgtttgcttatatgcatggagcgactctaggcgagaaagttctccagaataatacattgacgtcaagatcaaggatttgtg

SEQ ID NO: 19 - DNA, 1943 bp, Zea mays
  source 1..1943: mol_type = genomic DNA
agagaggacagctgggttgtactcgatctcgatgactcctgctccccgtatgcgagcacgtcaacgctagatgtaccgccgtcgtcgcgcggggcagctgtcgccccgcccgaatcaccgtcacagccgccacgcaaggtaatcctcctttccagcatccctatgtctccgtctccatccctctgtcgtctgatccgattcaccatccaattttgactgctttcgtttcagttgtgtatcggtatatacagacgctggtatcaaggcaacacacagcaattattactaccgccatcaatataggcgttctcagctcgccacggccagctttgtactccttccttggtttttggcgcacaaagaccctagattactctaggtagaagagacataaataaaaagctactgctagtactgctacagtactaggagtatgtaataccataggatcaacggccccctctgcagcttaagatcctaaaagtaaaaagtatagtaactgcgcgcctatactgtgcagaaacttatcctctcactgcacgccaattaacgcggagttttacctcaccattactacttgggtttgggggaagaaaaaggcgctgaaaaggcaccacatgcgcaaccagaagggaaggctacgtatgctgctgctgcgtactccgtgtgcatggactccgatccctcccagattttaagcgaggtgctagctacaagtgcagattgattggcgctgaccatgcacggcggtttcaaaattctccagaccgagcagtttttttgcaagcgttttcgaaattggctagctcagctgataaatatagatactataatgatggattggttagcttagctaggactataggagcccggaggcagcgagatgttattattggtccctcgaccgaacaggacaggatccacgacgacagatcaggtcagggtcaacacacgagagaaattaaaaagcagtgtgagtggaacaatcatgcatgcatggatgcctagctttggcgtggcctcatctgctgctgctgctgctagattcagatgcatcccgctcccattatcctgtccaggccaggggtgccgtgacagtgacaggaagaggttgttgctttgctttgctttcctttcctcctcgtttaacttccccttttgcccggaccaaacggcactataggctagctagcgcccacctgaccccgccgctgcataatttgcacacccccgcgctccggccccttcatcagccctcttttttccccctctcttatgcacgctaggttccgaaattttatctatttatttatctatttatatataatttgttttatccacagaaaaatcggtagcacatcatcaacagcgttctggggggtggtcggcgcacgtgacgccccccgtccggctgggatgacaccacaccacacgccatcgctatgcatgctgttgctgcggccgcgtgagcgtgacacaccgtgcagcggtcgttttctcttttcggccgtccctccctcttcttttcggccgcccctccctctgtgtgtgcgcgcactgtgcagcatgcaccagtcaccagcagccgccgctgtttttttttttgcctgagaaagtgcgtggctgccgcgcccggcccatggccggggaaatgtcctttccttcttcggcctccgtccggcggcgcacgtcctcgtggtgacgtgacgtggtgcgtcatccatgcacggcacgtacggggggaggggggggtgcttgagcgcgtgttcggtgtccccgccgccggcagacggacgagatggttgtggtgcgtgcggcgccgtcgatcgggacctggatggctagctctctgcctctctcgtgctttgtgcagaggcatgcttttacgccggcgagcggcggtgctgcaacagtgcaagtagctagtaggtaggtcggtgata

SEQ ID NO: 20 - DNA, 1569 bp, Zea mays
  source 1..1569: mol_type = other DNA
gaggacgttgctggagactgacttagtagcgagtccggccactacacgcacgcacgatggggtgtgccggctattgcgaaccgatgcacgcagctatcgtctcttcggctgttcggcaccatgtgagagctgagctatccctgagcagctgcagtgcacacggcacctgcgagcgtttcttcgaccctgtgttctgtgatcaggtcgcaaatgcgttgtgggcagaaagaaggcacaggcacggccggtacgcgagcctccggtcggcggtggtagcgcgaagaaggcgcatggagcgtacgggtgacggcgtgggggcagcgaggccgagattgccgtgtgagcgaatctgaagcagtacaccgtgatccgtaaacgatggcccacttcactttgttgtgtgcatgaacgcacgccgacacggccacgtgccacggcgcggtggttcgatcgttcactggtttggtacttcggttcggtccggttcgcttccggccgcctaacctgccacctgccaggcagcgggcagcgggcagctcggacgacagggctcgtgagcgcacacgctggccatctatctatcttctccagcaactcctgatctggaggcatgagcgcggccggctctgttcggccggccgtgcgctggaaaaggggtctgcagatcggctacataccctaacagcaaaccgatgtgatgtacctgtagcatgtcattggcagcctgcatgcgcgccatcgatccccatgcattgacattgtgattgtgatgttgattgttgagactagtcagtgacatcgctttcaagatgtaatttcgaaacctattttgctgtgatctggtcaccagccggaggaataaaaaaggggtttttataggcgtgcgcctgtggtctggctggtcacaggaaactcagcgactctttctggctggtctctcactgactaccataccaagcactagaactactctgtctgtgctgaacaattctttttctcacctcctcaacggtcaacacacagtacaggctgagacttgttcattcttcgccggcctggaagctactactacgtactataaaacaaagcacgtacgcattgggcgatcggcggcgtcggaaatggtgctagtagtagtattcaggagctagcgttgaatccgccgtcactctcgccaccaaatgcgctgaacagttccgattccgctttgaagctctcaaacttcacacgtcgccatcgccgccatcactgcgcccggtcacaatccctagaagccttctgcacaacggggcagcctccctctgcctcctgccgatccctgcgttcatccaaagatctcagctagctagccctacattcccctccgctgcacattgttgggcgtgcagccctgcctgcaaaccgagaaatttgctcctgtggctctcatgcccacatgcacaaccacgtggccatgacctgaagcccccaacaccccaccccacctagagatctaacgctgctgccgatatctcatgcctagacctttttctatgggatttggtcttttgccttctagccatattctca

SEQ ID NO: 21 - DNA, 1750 bp, Zea mays
  source 1..1750: mol_type = genomic DNA
gctggacttcagcgccataggtattttcgtcaagctcgttaccaaggtttatgacattgaggtcagcgccataggtcatggtgttaccatatcgttacgtcacttctacgctaacatcttgtcgatggtgtcaaactatgtaacctcgacgctatagaacatgaaatagacctagagtctcgaatcgattgtagttttgtctcaagtctaaacatgtgttgttgccctcttgttttgtattttgttcttttttttgttacgagagaagagatttaaaaaaaaacacaagaattgacgtatctgtaacgagcagagtacacacgtgggctagctctccgctgaaaagaatacgatttacatacgtgtaaaggttgtgcccactcggcagaaatttggtgatgcggggccaggtcgttagcctcggatgcagcctgcaggcagcctgtggtgtggtgtggtccaaaaagggcgggaacagaaacgaggggctggacgcctggacccatggatcaggtggtggtggtccgtgtgggcgcaagcaccagtacagtacagtacagtacttccccccccgctcctgcatgcatcgtcctctgtaaacacaaggctttacccgaaagcacaagctcacctaattaagctcatgtacgcttctggcgcgcacaatagacacgcccgtacgcaggagcacatggcaccaaccgaatgatttgagcaaccgtctccgcatctggaatccattccactcacccaaacagagctccagctccccctctatccagcgagctggacgggacgggacggagcgtagactagcagaacagaagccaggcaggtcgtccggtcggggtcctttccctctttctctccgttttctccgctggggaaaaagaaaatcggaaaatgacgctccacggaagaagcgcgcgagccgatggcaatggttcccgtcagcgtcgagcggcgatggtccccgagactttttccccccctcctcccctgcgtgccgcacacggccggaacggtccttgctgttgcggctttctatcttggaacagcgccggccggttgaatccgccgtgttcctagtgcaagttgcagagcggagcaaagcaagggaccttgccgcaaaaaccgtggcggggtgtcgtctaactttgtccgtcaagggtcgccgtcggccttgacaaaacggacagctgctgaccgtgacgagttagaagagagagagagagggagatagaagaaaaatcacccacctccggacctccccacacgaaacgaaaagctacgacctacctctcttccagacgtaacgtaagcatgaaacagaaagcactgcctgccggaaaaacaaaaacaaaaacaaaaaaaacccgaaagactaataaataattcaccgctcctctttcgcatttctccggatcttgtatgcatgatgtgtgtgtgtgtgcctgtggataattggacgcactccaccctacagtctcctctctcagcttcgttcctgcgcccccgtatcgtatcctaatcctaatgcatgctcacggctgggtcccgtgggccaccaggtttttaatgtgcccttctgtagccacacgcggaggggaaaaggaacccgcaagaaatgagagtggaacagaaggcgtctatttttgcacgatggtataggaagcctgctggcgggctcgacggtgcctcgatcagagcgtacaaaatgaagcgtgtgaagctgttggagttgacctac

SEQ ID NO: 22 - DNA, 1942 bp, Zea mays
  source 1..1942: mol_type = genomic DNA
gctcgtaaacctcttcgttcttttccatgttggcggcattccctttgtcgtcctttgaactagacccttatgaaaaataagtcgcttctgttatagtagatgggtatctagatagatcagaacagggatgcccacttttgctttcacatttttttgtccaccacaataataagcaacattgtgcctaaaaatatgttctccgaggcaaaaagaagttttagcctagctagccatgggttgcatgagctctgacaaggccttttatggcctagtcactttggttcgcacgaaaacaaaaggataatcttctatagtttttaaatccaattaatcaaaaaaatcagccgatgcaaaacttgtagctatattgcaatttgcaaagctaataattagtgtaaaaactacgtaccttgtgaggcttacctgtgcatagggtgggctataggaagaagatgcgtcattatctaaaattaagccttaatttggatggagggaaaggtatttgtggaatgtattgttaacggtgtagtactccaatccatgctacttaaataatatacaaacggacaggataataagatatcaatcatatatactccaaattaatgattggcatgaaacaagttaatcaaactaatgtctgctagacaagataaaatgttatcaaatgcaaacacgtgtgcgattgtcgtcctcacgcgcatatcgtcggccttcagtgtttttttctttctttacataaaatattaaaaaaagatcagaggggaattcagggagaaatggccgggaacaagactagccagaggaataatattgagtcacaaatcacacacagaatgcacatcgatgcataatatatgcgtccgcctgtcggtctcatcgtcagagtcaatcatcacctactgacgaattaatggacaaaaatcacatcttgctatgcacaaaccgggacttgctaccaactctatcttctctctacgcataaacaacatgtaacccacaaattaaatgacgatataatcgcccagttcatgcatgagctaatattgtttttggggggtacatggaaaatcccagtacgtgtatctttttcggtccagctgattattgctgatgaggggccggggccatcggatcaacagtcgcgtctctgaaaccttcactgggatgtctgatgtctctctcccccctctcattctcatgagtaaacagttcaactgcatacatggacgcgagaccttcttcttttctcgcagaagattgctcctttccttgacgcagagattaaataaagcctgaaagcgatgcggtttagatggagttaattagagctagaacatgagttttggaaagtgtttgtgtagtaggccggccagcagcaggggtagaatggataggcgcacgcaacgcaaccgacaagcggcacacgctggtactgcagcctgaaagctggctgacaatggtggatcgagctactaggtagttagctagggcctgcagctgcaactaggccggtgccggtgcttgctcgttggacattatatataatcctgcaaactgcgatgtgccgtattgaaataactagaaaacaatcgctgctgtttgcgaaggttgttatagattggatgaacaaaagtgtatatatatcgtttctttttaatttgtcatatttgtagcacgttcgaagctttagttataaaaaaactaaatggtaaaatcttgaagtatgtattatattaataaggcataacacaaaggaaatgcatccttcatttattggcgcaatgatattgcactggtagattaatcgttctaaatggaagaccctaaacacacatagctagcagcgtgaccttgaagggaaaggagtacgaaaaaaaatgttgtcaagatgacaggaatggtagctggctagtggagcttctcggtctcggaattcatc

SEQ ID NO: 23 - DNA, 1574 bp, Zea mays
  source 1..1574: mol_type = genomic DNA
ttcgtaataataatgcaatactgatggagacgtcgacgactggacacagattcatatataataatgcaatactaatggggcgccctcagtttcagtctttgcgctggaaatggccgatcattaaaaaaatttcttttgtcgaaattcatagacttgcgaaacgatcttgaaatatacttacttcaccatgttcctgcacccaaaaaaaaaagttctcccattcccatcctcttccaggaacaaaagcacagctaccctacccaggtgagggctgagggatgtgtagtagtactgtccatccctgcatggcggaatgggcgccggagtcggcggcgcttcgagaatcatgcgtggcaggcagcgagaactccaatgcaaggcagcttgctgccatcgattgccatgactgaaacacgcatgcatgcatgcatgcagagttctagtgtctgcaatggacaatgatgaatcctcctctcccctgcattgcaattgcaaagcagcatgcaatgcaatgctctagatcttccggcgactgggcgccggcgaccacgcccgcccgcccaccaccaaccatacgcatgaattttaagctgcccctcatcaaccagtcatgagtcatcattgccatgcaccccccccccgccgcgtcgtccgccgtgcctgccatgccatcgccggaccagacacaatgattcgcccatgatcatcgccggaccggctagtcgatgtggatcgaagcaacgtacgtactgtacgctgtgctgcagtggcaccaccactgtatgtatccactgcaccgcttgttgcgcccacaccaagcacttggtagtttgcatgccccgcagaggtgtgcaggccggccatgcctgcaggctggctgcagccggctgcatgcatcggccaagcttggctgcagagctagcgatgcatactgggctactggcgtcgcaggcggcggtgatgcgtagtgcggcagtggtcgcggtcgcagccggccagcaggaagcgccactggggttttggagagacgtgcatggcgctttctccgggcgctagctagcttagctcgagattgactggcaggctgcatgggcaggcagtgcgcctggccgcgcctgacgacggctgtgcctcggctagctcttgctgccagtgccagtgccaggcaagctgacgcccggtttcctccaccaccggccaccgccatccaggatcaggttcagaagaggagagaaaaagtgtgcatgatggagaaatactgtgagcttcagtttgcccgatgtcacagcagcgcgcgcggtgggcgagggaacaggaggacatgtatgtgctcagctccatgacccggccctccaactgtttacggtcttcgtggccctctaacggttacagtgtgaggatagcgcgcgctccgtcaggccaagtcaaccggatgagtggacgcagcaaaacagtgttgcatgtcacacaagaatataaaaaaaaatttcccttcgcggacccctttttattcaccggtggtgccagtaactctttcctccctatgcttacgtatcaatgtgcagatagctaga

SEQ ID NO: 24 - DNA, 1601 bp, Zea mays
  source 1..1601: mol_type = genomic DNA
taactaatatgcctcactaatcattatatacataaacaactgattatttgcctcccagccaatgatataagaagtgacaaaaaacatgtaaaatcaccaccaggaagaaaaaaaaactttttcaatgttccagtatataacatatatactgacaaaagaccgtatgagaatttggcccaatagtccctccaatttgacgtgatcaatctaaaccacttgctagtttcactcgataaagaagttacccaatttccaatgccacatataacatatatggtacagcacccaaattatttgaatttggacataaggaaacgttacatgcacctcaagaactaactgatcagtttctggcctgaaaacaccagattacccagcaagtattggaacagtcctgttccagttttaggggagttgccaatagctaccaataaatgaaattgactgtccaagcattatctgaataacaggctcagctgttgaatttctgacaaggtgaaaacaataatcttactcagtatatgtcttgttgaacagctcgcacgaaccaacaataccaggccctccaactttcgggtactgaaatagtagaatgaagatgatgtggaaaatgcagtcaaaagtcttgtaaattataaatttttagtagtctggaaaatcttgcgtccatcttttgaataataatgtgaagaccagagccatgatgtgcaagattacaagatttgatatctgaactttgaagtgacatattatcaatatacactaccaaatcaagttcatttgcatctcaacctcatcgcacagttcgaaaaacctaagcaggtatatataacatggcattctatagtgtatggctttatgaactactttcttattttgcaacctacttcctgatgtagtttaagataatattggtaaaaaaatgaacaatgtacaaattaatccactgatccattatcaagcaaaaaaatgttgcaaatgatggaacttgcctgtgatactagtgaaagaaatttgtaggtatccaatgcatatctcaccaaagcctcattctcccctggatttatagtacacctgcaatgcagaaaagaaagacacgagcactaataaacattattcactaagctctgctttataatacatcggcaatgcagagaagaaaggattgagcactaacaaactattcactgagttttgttgtttgtcctttgaattggtaatcacacttaccactataccagttcgtattgtatgatatgcagatactagataggataacattgtaaattaaccacatgtacataaactaggattacaaatcaagttcttcaatcgaatagcaatcaaggatagtcctgcaatatatgaattagttgatttcttgactaatgttaaatttcttgactacatagttatgactggtatttatgcagcaactacaatgaatttctgatgataaatcactagctccaatgttgtacctggtcctgtcctttatgttcaatctctggagcctggataggacaaattaggtggctttaggagctcctaggcacgtccagtaagtgtttataggcacgtgagttgtattttctggtagg

SEQ ID NO: 25 - DNA, 2872 bp, Zea mays
  source 1..2872: mol_type = genomic DNA
tttaccgcagcatcacgcccacgccggcctccatgctctcgatcgattattatatttcccaccgcgcgcgcgcgtccagatgctgccagaatcccatcattcatttccatgcccggccggccggcctgcgtgttgaaaagttgcagctagcacacagtatcgctatcatattcaggttcagccctgcagttcgttgcagttaggctctgaaacacatgtagatagttagatagatgcacacacacacaaatgcatgcgaggaggatcatgcatgatgatgaacagtcgttgcgcagtagagtgagtggagcacacgcaatcagacgcgctgatagctcctgtaattgccgccatcagtgacatgatgatgatgctggcgcgttcattaattccctgcagctggcagatctccgatcccatccgtcgtcgcttgcagtggctcagcgagagcctctgatgagcttaagctcttgttcaatatgcgctcatgcatctcactgtattacatcatgcttactcggtgatgatgcatgacgagaccgggcaaaatgttgctcgcatcgggaatgatgaggaatagtagtgtatataatgatgcgcatttagtacagccttttcgatttgtttagtcttgatgctcacgggatcgatgagaaaggctgcaggccgggtgcatcagctgctaatcatcaccttgtcttatattactccagcgatcgatctaatctaatccgatatatgtatgcggtatgcctagctttctagttagttatgtaaaatgcagctagcaaaagacacaggggtatactgatactagtatacaataataatgccctaaataatgcagagcacatgtatctaatgcagcagaaaggcaaaagaaagggtaaagaggtataagtagtgtagtgggtgtgcatgggggccaaccacaacagtctgaacaaacctcttgcattctgcaccatccatatgcatatgtgcttgctttcgagctcttttggaacaataatctgcagtggcccgcctacacagtcactggaccaccacagtgagaacaagagagagagagagagctttggtggtctggtctctgaaacccatgtgatcatcatatccctgccctggccactaaataatataatttcttgttttccatgaaggatctgttcacgcgatgtagatcaagcttacgagcatgaaaagtggtcagaactcctacttaataaggagttcttgggatatatatttgaccctacttctagtatagtaactacatataataaacaactcagttgaaaaagtggtcaggtcaactctgctggctggctgttgaggtttcagggtcatacaaacaagactggtgaaagctatctgctttacttgtcatggcgccatatccccacgatcgaggactttcttttcttgcttcttcctcttccttcagtgctagctagttctgtacgtctctctgccccctgggcctgcgcatcctccattttgccgcgaaccatatgcagacaacgcgcgtcaaaataaccctagtcgtttactgctcttgttggtttccttggattcagctagctgtgcgtgtgccccggcccccaaataaggtgagatctgtcgtctgaaggcaggcatatgatttgccttgatttctacacccagcaggccgggggccggctccatcgagagctcagggacacagagttgacatcagtgtgtatactttttgtacgttacacttgagtgcttcatcagctagctgctagctactccactcctagtctcctactactatatattgcggtacatgaccatttgccagctgctgtaatgacacaggtgctatatatatttctacaaaccaaaatcaaaggaagaagaaaaaaaccagcttgattatcatatgctggattaaattatccaatcatcccacaatcatgaaatagttaactagagtaacgtacggtaaatgcttattattaattttggatcatgagtaattaaacttttttctactgtttgtacgtactttaacgtagctggcagctaaatcgggtttaaaagcggtatcttggtctagctcaaaagggtcacctagctagctttattttccatgcatgaaaaatatgaaagctttgctcgatcttttataatttagaaagttccgcgcgctgacttgtttgcatggtgtgtgtctttgcatagctctgctgccgcattaattttgccgcgcaccacgcagagacagtgcgcatcaaaatgcaagttctggtggcaatgcttttgttatttactttgttcaaacaaaagaaggtaaaaatgagtcgacgaaatggtacgtaataaatatctgctcagttgcaaggcatgatttgccttattggttgctttgcttccacccagctagcgctgaacgcttcgagaggcagggacacatgacaggtttggtacacgtactcgagtgcttcatctggctagcaccgctggtgagatgagtaacttttacttaaacctaggatatatgatcactcttatagtagtattacatcgtgcatcaggccaccaggttgaacgatacacagtaaaaaataggagtagaggtcaataaatatttatattccacgaataagatatcatttttttttttttgctgataaatggtaaaggagaagttagaacccctaagggacataataaatatttcagatgataatatcgttaacacacaagagagtaatgtccaagatgaactaattgacaagagccttatgctattaaacttattcatcccttgtagttaaacagctttcattaaagagagaaatattatcagtggtaaatggtaatgatttttgcataaaatttt

SEQ ID NO: 26 - DNA, 2642 bp, Zea mays
  source 1..2642: mol_type = genomic DNA
gtgagtaaagtaaacttcataaatttgaatgcataagcacctagtacgcaaatacatgcgtgaaaaataagagtaaaatgtcggctccataatcctgcattagggataaacatgatatgttcagggaattctaacaaagaccgatagatctacacatattctcacctgaattggtgtaccagtgagacaccagcgccgatctgcagtcagagcagccgcagctagggatatcaaacttttagaagattttatcatgtgtgcttcgtcaagcacaactctgaaccaatgtacagagtacaaagccccattttctgttgaaccctagccaagcagatatcaaaagttcagtcaagaataagtttgcataattcaaactaaatattaatagcaatggacccagcagttccccatcattgcatccatactttggtcaatgtggctatcttgtttatgcatgtatggaccaaatttaagcaagccatattttaccaataagattgtgtaagtatgacatgaaggatcaaatgcagaaacatggtgggaaggacaaaagacaagcaaatgggtaatgcattatctgtattattttgataaaaaatggacacaagtaaaagtagaagatgtcaaatcaagtggctatagtagtaagagtatgcaaatgaccaatcaatcgcatggtctggtgctatctaggttatcttaattcttaaccaatgaattttttacaaaagcttgaacataataagagtaaactgaaagagatataagatctcagagcttacatcgattgaaaactctgatgacacaacaccatatgtagtcaggacaatatcactctgaccaatgatgcttgcatcttttggcctgttttgtccatagtgaacatatatattcgcagtgcctggcttagtatgagcttcaatctctgcctgttattcataacatcaactctagtgttcagaaaaatagttgaaagaaacacttaaagatgaaattggtaaagcaagtcaaaattatgttaacctcttatcttttacttttctgcagcaaataagcacacatataatgtagcataggcaatggtggggtggtgccacaattagaactataccttccactgactaattagtgtcattggacagataattagattgcttcctccaataagtggggccttaggtttcttgtgtttgctaaaagagaaagggttagcaagcttcttcacagcatcatgcgattcacccaacccactagcttctctaggggtctgagcagcattctgagttgtgatgcatcctttgctagaatcagaaagaagaagagctatcgtcataatagtcttccccagtcccattgcatctgccagaatcttaatttttactccagttaatagaattgatcgtaaaatcaaccagaaacgatagctatcatagaaatgctaaacataaagaaatacaatattaaaaatattcttactcctcctctagaaagttgtaatgtactaggaaattcagttgtagcatcgcctgaaaacacgttcaagtacaaaacgagttccctcctgaaataaatgcaataaaatatgaaatgtaatgtccaactattgccagaatggttattaaaaacagctcatataacacatgcatacttatcctcaagtttgtatgcttcccaacaagggtgaagggttgtagcggcatcctgggaagaactacctttctcaagctgcagcatccaatgaagtgcctgcttttgataagagcgtagatcacacatcagagaatcagggggagccctttcctacaaaatataaattcagagcacactgaggtcaggataatatccactagttgctgaaacactgcacaacgatgggctgttgaattacctctagtgcacagctgtctgagatcccaattatatcatccaaatctgaatctgaaacagtttcttcaccatgatcatcttcgtttccatcagaagacaacctcaatctgccggatgtcagctttgcagcaggagaccctatactgctctgttcaggcagaaaaaacataaaaggccaatatctattattttgatagcagtgtaacgaatgaagtactaaactattacctttgtttcagttggccgcttcctggaataaagatcttctggagtaaaagcggcctgttttcaatttatgtcaaaccaaaaaaaatcagaccatactaaaatgttgattccagagaaagaggaatgcatgtgacttaaatggacttgtttcttttaatgtctttttatacctttatccacattctaaaatcatatgttgtacaatgttggggtaaagatatataaatggttctcatacttcttaattttttgctttcagtgttcatcagtgactgttcaaaaaatatttatcaacgctacgctaatttcatgataccaaagaacacctaattacaattgtacaaactttggcaatgcatatttatacattcacctatccaagagctttaaattggtagacttttgcagtgcatttgcactagataataaacgagcaggggatgataagagcgcacagctcaccttaataaaaggagcaagtcctatcaatttgaaaagtgcaggtagggggtgaaatgtggagtcat

SEQ ID NO: 27 - DNA, 1657 bp, Zea mays
  source 1..1657: mol_type = genomic DNA
aatgattgaaataaccttgaatgacttaatatcatagtttcaatacctattcctatccaccaacttgttctgatgagggttatgacatctcagtgtctcgctcctgttgttcagcaataggagcctttgcaccaacaccgcctttgccacggggtggtctcctttcttcaccgtcatccccatcatcatccccccgtacacctcgtcgtccttaccctcatttcattcatccttctcctcatactagtgctagcagcggaacatttatgttcctagtatataaaacttaaatgagtcaatgtcacaagaaacaatacgatgcaattcaatagcacaaaaatgctattcctattaataaaatgttttcactgaatataaaataagaggatatctgaataggtgaagcatcaataaaaataacttaaaactcactgattcactatccttctcctgagtctcacgtgctgcatttgttgcctcttaccaatcctcatccagctccatatcctttatatcctcaccattaggccgaggcttcttgaaaacccgcttctacaaaaaatcaaatggcgtgaaaacataatcggatgtctaaattcatgaaaattataacacaatgactaaaactcacaattgtgtgcctcttcgcacacattgccaaagtagaatatccaattgagccagccgacactttgtcagaactaataatagctctgatgtctccaataagcttctcatgtgccctctttaggaccaactgtcattcctggtattttcctatgccttttcaatcttcagctaattggcttggatatttaccagacatctttgaaacatctctattacaatctctcaaagccctcaaaacctcatccttacaaataatgtcaacaatagtagcaatttctcctacaagtagtggctccaaacaagaaagcccatatgcctctatttgtggaacttcctgttgagaaacaggaggaggagtaggtctactagaaggtcctgccttcgtagcatcgtgtgtagaagatggtctagctcctctcatagcaactattttccaaccatgtcgtggattaggaatatacttcattatgccatcctttgctttaaatatgccagctagggaagcgcctccccaacatccttctgttgaacaccgtgatgaacatcattatatatagtctcaccgacatccttcaactgcaaatcaaaaagcaaaaaaatacaaataaacttcatacaaaacgattgaacacaattgattttgtaaaaaaagatgaaaaagaaagaaaggcatgacattactaaccatcaacttgccataagtaaaagcatgagaaacttggtccacaactcgatctgcctttgttatcctcatgatcacatcactagtgaaaatatcgagatcgaggtgtgcccaaagggacatcgacaccagacaacaaattatctaaaaagtaaacatgtagatatacaaaacaatataaaaatatctaaatgtcataatacaaaagctaattatgtgaaatgattatgaatcataggataatttctaagaatttatagaatgacttaaagtgacagaaacaaactcacatatatttattatgactaaaaaacatgatatatgaaatgacttaggtaggcaaat

SEQ ID NO: 28 - DNA, 1912 bp, Zea mays
  source 1..1912: mol_type = genomic DNA
acattgcaacaatatgtctatatatgatggtttctgcaccgtgatgagtagcaaagccaccatgcatgaccgatatggcgattggcgaacctaaaaggaaagcgcgaacgcgaccactttggcttggcgtcttcctcgtctttccactcctacatgcaccccctattccatcgaggcatcatgcatgcatgctgtcctgctcgctaaaaagccatggatcatgcgatgcgcacgagagccggaggacaaagcaaaggccctagctagctagctagcaagcttttgtaaagccgccatttccgattggcctctacctcgaacagatctcatcgatctcgatctgccgcctctcccatcaagctgtcatgagtgacaccgtcgtcgtcacatgcacacatgcgtggcgggcaggaaaaatgtgcatgggcgaacgagcgagccgttggttggaggcccgccggccgtccaagcacgacatggagatgatcctagtcgtccgccccgccctctctgatatgtactagaggcaagtagtcctagcttgcacttgtgtgtctgccatgaacaaacacgacagtgcatgcatggttgcgtatgtacaagtcttgcatgttgccatgcatagggtaaaaaagagagagagagagctaaaagtgatcatagcattttgaatgtcgtagctattttcaatttcttctattttgaggaagctaagctagccacagatttccagatgctatagggtaaagctcaaaatgctagcttctctcagctctgtctcttgttctgagaagttgttgcacgtactaggagaagatcgattgaactctagctactggtaaagttgcttttgaaggtaaattaaagcctaaagctaactgatcagctgatttactaggaagctgagtctgcagcatggtgtggtagtgtgaagagagatatacagtgagagcatcatatcgatcggcatgcatactactaatagtgcttcccacgttgtcactcgctttttgctctgccggccatattaattcgttactctttattttgcatgagttcatcagatcagttcctctctccttagaccagctacatacagacaacggtggcgagagtccatcaccatcagtatctattggtatttaggatggactaaggtcgttcgtcagtgcttaatcacaccgtgcaagcaatgtcattgtacttacctttgctttatgacatgcaacagctgcatgcgctgcagatgcattcacctcgaaatgcatgcatgcaccactcgccagtcgtcactcaccagcattatattgcttgtgtcttggttttcggtcaccccccctccccctctacaaattactatagagagtgagtgagattttttttttcactggacaacacgacgagtagtagcataagatatatagcctaacataataatcccattcccctagaagaaccctatcacttaaccaccgcccaatgcaggaaccgctttatatttaaatctggcgatattctttactgtctgatagttcgtgcctgtgatcgggaggtgtgcctcaatcatggttaacagaacgaacatggggcttgtgattcctccttagcttggccaagagtcacagtcgtcgtcgtcgtcagtcagagggtaggctactcacatctatgcgaactactgacatcttttcctcgattctttttatcgtcatgggaacatggagcttttcatgagtctactgggctagctagctagctcgtacaaccatgagattggatacagcctataccggcctgtctaggaggacaaatgcctagagaattttggaaccaaactcgaaagcatgggagtagtttttcactctcaagattcaagaggccaagtagactagtagagtgtagctagggagactag

SEQ ID NO: 29 - DNA, 1275 bp, Zea mays
  source 1..1275: mol_type = genomic DNA
tatagtcgatctaatgacatagaactgtactgactcttgaaagatttcctgaacttttgtttggtaaaatatcgcgaggtcaataaaagctataagtcatcaccccattaggatgaagcaaagcaacatagacaaaacaaaagttaagagacaaacagcaaggacactcacaggaaccatgataatcattatattatattagcgtgtatgttataaaagctaacctgcctgcccccacatattacatactgtagcataaaatattcaacgaattaaagcaagctaggtttaggaccacatgagcagctctgcctgtccctgccgcggcgtccaatgggaagcagccataagcaacgcgctgccagtaccgccattcagatttcagagaggctcatgatgacatcacacatctcactttggatgggtcccacttcgccactaccaacgcctgcagtgggtcatgcatgctgccatggcagcatgtgggagggggggctcagcatcaggccctcatcacacagctcgtcaagtgatcggccggaatggagacggagtatctttttccaatggaatcccccctgcacgaacaaaggaatccagtgaaggcatgtggccgcatcagacattgtatcagctagcggttagttaataaaggagcctcagctagcagggaatctttctgcactggcatttgctgccgctgtgatcttggtctttggtcgtcttgccttgcgcggtggggtagaacaagccagcagctgttagcagaggtggatgaaaaccatggtccttgcatgcacagagagagagagagagagagtactcaggctggcagcagcagccagcagcagagcgagggtggtcaaggacacttgttgagatgatgtggagaggtccgtccatgtccatctactaccagcctgcaggaactctgttcctgattcagcccattcaattaagttgttagtagtaggagacatgtcatcagtcgatgtgcagtacgacatgcatgtgtcttctgctactgctgtgtctccaaaggctcatggtttcactgtcacaccgaccagtgctgctgctgatgcaacagttgcggtggtacttcactgttgcatctcgcattcggttgcaataccttgacttcagtttagatctatccaaaaacacgctgcctgtcgatatcttgccccgggaagcatggagtgaacagaatcaccacaccagggcgatgttcatctacaagcgtggccaacgaatagctgtttacgtacaagc

SEQ ID NO: 30 - DNA, 2102 bp, Zea mays
  source 1..2102: mol_type = genomic DNA
attttcacgcgaatatgcccctaagaccctgtccacttgcccttgccttctcttgcacagctccccgcctggcgcctgccgcctgttgaccggacgcgtgcgtccgtagcgtggcgcacgtacggagtactcgcagcgaccccacccgaaacgagccaaaaagaaccacaccgcacaagtgccaagtgccaacaccgccggaaaccacctcgtgctggcccatcccgacatcccgtcccgtgctcccctcccctcctctcgaccacctcctcgtcgtcttcctcgccccctccgccacctccgccccggcctctccgctcccacccccgcggaaaccctaactccctcttggcacaggaggcctatgacggagattcgccgcgcgcaaggagggagtggattcgcgactgcgcagccgctgccgtcgtcctagggcgcgtcgagcactgccaccccattgacgggcccatccccacgacatccgccatgttcgagggccacgtatgatctcttacccttcctccatgttttcccattccttcagcacctttgatcgtgaccatttttgatgcgcgatatggaggaaggccgcgtgattgcctagtggaccgatgtgttggtttagtcgtggcgttgttctgtccgggttaccaaatctctgtgcagatcatgttcgtatttggggatagtttggagggttagaattcggtgcgttgatgattgacccttggtcgtttgcgttatgatcttcccttgaggaagaattgcatgttgatttatggaggagcctctaaacgggcaagttgtggacttttggtgcttatgtatacgtttgttcacaattttatagttcgactgaattccaactattgaggaatctgtttactttggttgagcaaaatgccttatttcatttggcctacgatcatttaagtagctcatgacacattgtctgtgatggttgcatatggtgatctggcagcaatgtgaaagttcatagctgcggcatttctttctttttaaattgaaatctcctagcccctgttacctcccgacaatatgcacttgttgccctttaaatttggatattcttctttcgtgattgcatcttattctcaccagtgaatgcagttttgagcatccacactattatggtgagagctatgttttgtgccatcctactgcactgcttgggatttcattgtgtcttttcttttgcgtcattatgtaagaaacaatcatgctggtaaatagtggagtcgtgagtaattcatgaggttttacagcaactttgctcattcatggcaatagacacgtaccacttaaatggtgaatcatgttatgcactctttcgctatctataatttcatatcgaatttctattttttgttttcctttttttgactctttcatcattcatcatttgtgttaagttctgagccatgacatagttgtaacttttttctacaggtgctgtatctgctacgcaaatatttgggtgaatatgttgaagggctttctgttgaagctctcagaataagtgtatggaaaggtatatttctttcataataatgttcttttctattgcttaaccttcatctcctgcaatccgattccaattatttcatgttgttatatatgctttctattctgctgttgtttgaccaagcacactctttccttatttagacaaaaaaatatcaaatctcaactttattgtattgtttatttgatactaccctgaagttagatgaagtgaacaagttgtaacttctttgttgtatgaagtgggaatgtgggataggtgttataaagcttgccgattacgtgcatacagagaaacagtcttattcaggaactcgaggattgcctcccacattttactcatgtacatcccttgcttggctctaagttgagtcatataatggattactgctctcctgcgctttcgaaaaaaaaataatggattactggattctcaggcctgatttggccatgcagtgtctctgtcactcttgcatgtatgctcatggatcctgtttctagagatgaaatacccaatggtgttctatctccatcttgcttttttaaccgtaattttg

SEQ ID NO: 31 - DNA, 2367 bp, Zea mays
  source 1..2367: mol_type = genomic DNA
tatggataaataaatccggacatagtaagtgtcacatatgttatgctagataggacagggtagccgcaaatactttatgaacaacggtgttacggagtcagaatagtatatcaatgtatattcattttcctcttatgatgactacttcacggacttgtattgtattattaaaaatcaaattaggggataaaaaattgtgattatatcaatcaaaactaaaggcaatattagaaacatacaaaaatacatgatacatgacacttcttagttcttacatcctcatgtcataataccaaaactaagggtcataatatatccataattgttcttgattagcaaaaaaatcataattgttattagaataatcatgttggtacaaaggcttgattatgtcctataaatgaattatagcccaaaagttaaaagaaactcaaaacaaaacataaaagatattaacaaactacaagtaaatagttgataaactatattaaattgttggcatagtgtcactgctgcatgtgcaaacatctgctgctgctgtatccgccgggttcttacgcatgtatgagacagtgatttcatgagaaagattattgacacgaaatcagaatccagaaaacaataacttaaacagcacaaagggagcagcagtgtccttgttggataccttcgtcgggaaggtgatagatcacataagaatcactaatctaaaaaacaaaacagtaagtttaagagcaaaacagcactagtaccttggctagactgtacattgtacttgaatactctgggtgaccaacccccagaacacgtccttctatctacagatggcaattaaaaaaagataaattgcatacttcaacaagtgaattacaagcacatttacatttccattgttttgctaccatgctttgatgctttcattgaatgggtacaggtgttaagcagcagagtgacagaagatgacaacatatattcgtatagttctgaaaggttatattttaacctatttgtatgcgatcaaccaaaaatggacaattataagtatggtcaagtaagatgatagtatagcataactcaaatattgttattgtggaatatcacattggtatcaattatgacagaggcacaaaatttcaagaataaatgttaaacataaataagcaagagcacaaaaagttgaggtgtggtaccttcaaagcacgctgcaaaaggggggaaacaacaaacagaaaagggtataagaagcaagttcaaatcttcgaatgaaaaaaattaagttcaagacttggaatgaaaaaattaagtgcagaaagtatagcagcagttgacacaaagggtataatgctacaatggaaacagttcaattagcctttcacaacttcatgagaaaaatacatcaaggatctcccctactgtgacttcgttactaaatttcattctgtgtatcatgataatgatgcatatagtgaggactagcatgcctcaaacaaaaagctgcctacaaaaatgtccattcgctcacttgcagcttgaacagagtattagcatgcaaaattttgcacagatttacaaatttcttagattatgattcccatcaaaggtaacagatattgtcattcctacaaactattgcatgcggtctgttttcaaactgtttaatgatattcaccaaaaaagacaaaaacaaacttttcaaactgtgttgcatagcaaaattacagaagaaactataaaatctaaacaaagaagaataactcatgaggcaacaaaacgttacctcataacatgtctgggcttgagcaagcttgagttgaagatgatagcatattccaagactatgcacagttcttccaaccctacattaaaaaatatataatttgatgaattgccaaatttaggataccatgcatttggtgtttcaacagttcaacacataggatcaagcatagaaacacattgagatagataggaaattcatttagaatgttcatgtaggtgtagcaacgacttacatctgcagctttatccctgaaacttagcatttggaatgtgatggctatcgtgctaaaatgttataagtttgtcaaacctataacttctatggcactgtgctccagttcacttgtgtgtgcgacgggcctacaactgtaatagcactatgctacaacacatttttgtaaatatgtactgaacctaatctatgactatcaaatggtacccctttattggtagggtacatgtcataaaatgtccctggggcaagctgccatgtcccttcagaaaaaggggagagctagaaaagcatcaagcagaatcagagcaaattatcgaacacgacattcagggttcatcttccgattggaaacc

SEQ ID NO: 32 - DNA, 1942 bp, Zea mays
  source 1..1942: mol_type = genomic DNA
cggttccagcagcggggcccgggccgcatttggtggtgatgggcacaatcaccccggggtcggggcattgagaatcgaaaaggccagtggccagtggtgcgtgcgcgcacacgctggtccacgcctgtacggcggctcaggtgcgtgggggcacgtcggttatgggaggcgccacagcatgcatgcagtacatagagtggcgatggcgagcgctgggggcgtcgttgggggtgggctgggctgggcgggggacgctggccccggcgacgccacgcgacggagcacccgacgagacggcacgcgctagctacctagctggctgccccgcccgcccgggtcggccgatctccgttcccctgcagcaattcaacgccccgtcccgtcccgtcccgtccctgtggcctgtgggcgggctgcccgtcactcccggccagctgccacgacgtacgggcggggggctagctggcacgcccggggaccggactggagtacggtacggcggtggcgtcggcgcatggatggggtgggggtgggccggatgcttctgctgcttgtggtggccaaccacgcatccattggttcatccgatccggacaccacaccacacctcccccgccccgcacgcacgcggtgcgggagtgcgggactagcctgtgctgcgcgtggcgagcgctcaacgcccgtgccgcgctgctggtcgctggtcggtgccgagctgcctataggtggtgatgacggtgctgctctagtagttagttctctactactactcctagtctcctagctcactagtgaatgctggttgcagatccgtgtggtgcggatgcagtacggtacgggcgcggaggggatctcgcgcagtggctgtcggggcctcgtgggggagctaggggaatgggatctcgcgcctcggtgcggtacgctcgggtgtacggcggcatgtgcggcccttactcccatggaatcaaatcaatgtccgtcgtcgtgtgctctggcaccgagcgccgcgtccagtagcgtcgtcgtctcgtacgccttggcctacgccagctacgcgcggtcaccacgcacgggaacgggaagcagtgcatgtacctttggctagctagtagtaggcatctacccacgccatgcattctccgcgccgccgtctgctgccctagctcgtctcctctgctcacagtcaccggaatgcatggcgtcaccgccacgcaagccatacggagcagcgacggtcgatttcatcaacgacacgacggagcgatgaccgcaacaaagcacggtcggtcggtcggggtcctgagcgcgtgcgtgcgcgcgcaagcgcaagcgcaagctggccggcggatcaggaaatttcggagcggacagggacgggttggccagtcgacagggggcctccatgcacaagcgattgtgcggccgtggttcgctggcggtggcggtgcccggcgcccgcccgctgggcgatcgatcgatcgatcgccctaccacttctttgtgccgctgttggttgtgtcacgtcctcatggccggttggccgggccccggtcctggctcatcctgcgtccaagtgcaagccgcgagtaaagtaactgtgtcctggccctggctaccactttgtgccgctgtgttgcctagtgaccgagctcgcccagcggggagaacagaactgtgtcttgtgcgtgcggccagctgtgtggcgcgctgtggcgtacgtcgctctcccgcgccctgtgcacgtgtgtgcgcgcgcgcgcgtaaccacctttttctcgttctcggcaacagcccggcactgcaaatcagcaggcgatcaccgcccacacagctgatgccgatcgagcgctctgggcggcgacgcgcgctgttgcgctcgctgttcgtctccgacaaagaaaaggtggcgtcggtgttggctggtcatggttaatcatgtcac

SEQ ID NO: 33 - DNA, 1994 bp, Zea mays
  source 1..1994: mol_type = genomic DNA
agggatcctaactgcttgctttcgaatgtaacccaacagattcacgagataggctaccaaagagatgttgtttcgacagacaaaagaaaaagagtgggatatcttcgtctataataaatgagttagatggcgagatgttatcagaatacgcttgggatttaagtcacgggcctgacacgtgtgagacaaccttgccatcctccgacacccgtccgtccatgtctgcactgacgcctccgagagcgcaacctgccaacacacgctgacgagttcagacgagccgagcccatatgatatgcggccatgcccgtgccgctgtaaggacttctgaaccactggcattagtttatctaaccttattacattaccctgaaagattagcgcctgaaagatttgccttgctgctaaagcatgcagtgacattgcatccagcaaaggacagcacccaggtgacaaatgcatttctctggggggcgaatcacgaatccatggcaacctgacgggctgcctgctctgccaatactggtcacacttcagtgacaaagggcatcagaaccaatgcaagaaatggtcggtgcgtctgaagaaagatgagagcaagcaggcaggccgtcgtggtctcttaaaatcgacgccggtagacttttatttttatttttaggtggcttcccaagaatgtccggcacgcgccgagcttccatgccgtggcaggaggcgggcggggcgaggtgaaaaggagggcggctctcctctgacgctgctcctccaccttacaattaaaatatttgttgggtcgattgggatgatggcgtctgtcaacagctaatggaatcctcgaaagccaagtctcgcctttttttttcttcttctcagatctgcagtgcagtgcagcgctcatggaagccatgtctcgattgggatgattgctcttgctcgccccaggatccggatctttctgaaagttttgggatggactatcgttggtgtaaaagtatacagctcactgctcttttcatgttctatgctatatgtattatatacagagagagagagagagaggtgagtttggaggtttagataatggataagagagcttagcgatccagctaggatcagatcatcagaaggccaaaacctctgagagccttacacttgacaaatgcttaaggactcacgcttaaagcttcaaaagacaacaaagtggcagttggttctcaggtaaagtacaaatagatggaaacaagtcagttccatctgattgcaacatttggagggtgacaatgtattttgagcttgttttccaaacataaaaaacatggacaggaaaacacaacaaccaacgtcagctgtcacttctaacaacaggctaccaaacagtgtgacatataatgcaaggcagacacgcaccatttcaacgtactaccataaaaccagacccagtgttctgacagagcactagcacactgctacgtgtgcagttaattaacggagctgttggggtttgactgcaaaggtgcatccaacatccaagcagcaaatctgacgtactagagctcttatgaacagcacctgaattagcgtatcatatctgacgaaaaccaaaaggtactgtacctgtctctccgcttgtaaatgaaatgaatggtggttctggtattttgatctctgtttcttggagggcgtgatagctttgcagttaggcaaggcaatcaccgggggatgatccagtgcgttgttctaaccaaaagggcagtaaacccaaccactcgctagaggtacaagaagttcaggttgcctaggcgttctgtcgcaactgccaacagcaaagggatctgttggtgactgcagctggatctgactctctgagttgagataatcgcctactatatcctgtcagtgtcaggtccaataaacagtctcactcagatatttggtatcgcaacatgcggaggatattctcgtcgaaacgaaatgcaattatccgtggattatca

SEQ ID NO: 34 - DNA, 1898 bp, Zea mays
  source 1..1898: mol_type = genomic DNA
tatatttgacactataacaaatactaacatattttttatgcattgagcgttgctgactctcgtcaacatatctcagaccgatggattttcgaccggacagagaaagcaaaggcctgcagcctgccggggcgttcgtacgtacgcgctacttgtcatgaggcggaacgagcgagagcattgaagcgccgcgagcaggcaacggatttccccagcagctatatagcctgtagagaggcgctgaaaagattgatggatcgaccagcactcagcgctcttttcgatcgtgtcgcatgcatcttgactagtagcagcttgtttggatcggagctgcagctttattcatgcgtcgtcgtcgtcgaccgctcatctatctgcgcgcattatatagcatctgctagctactactagtgcgcatgccacgcgagcacatcattcggtcaatgcagcctgcacagctgcgcgtacgtgcgctgctgcatcgatcgacgatccgtcgccgattaattacgtatactctgggaagatagatatactgtaaggctgatgccttttttctgatgagcccattggatcgatcgatgtgcatgcgtgtcgccagaaataaagaagcatgcagccaatcaagctagtgtgtgagcaacgccgcaggcaggccaggcatgaatgcgtgatgcgtctattaatttgtcccatgcatattatttactgctgtgagaaatatggcattagcatgcacactgctccactcgccaatgggcacagttgctcatcagccatcaacttttgttttgtgtcagcgcatgtatatatgtggcggctagctgtgttctcgctatcatcaatatgcagcagagagagatagaatagatagattgacagctacgtactactagtgtgtatctacatagatgtacctttgcagtgcagagaagatacatacaactacgtgctgatatgcattatgcatgcaacgcactgcgcacctgtcagtcagggtacgtagcagcacatgcactacactgtaccagcaggttttttccttggatcatgcatctgtgtgtacgtgtggtaaaactctggatccgatccaggacgaggacgaccatatcacctcacctgatctacatacatatgccatgcagtgcaggattaattattggcctccatgcatgcatgaatcggatcccgggacaatatccggacacatgcatgcttgaatggacgtacgtacgtacgtgcatgtgtatatgtacgtgtatgtaattatgtatgtaggtgtgtatacctaggcggctaataaataaccggatgtgtatacttgtgcaagacgatttaattactacttagatatgatcttgcagaattaaacctactgagattagctaggtatttaggacgacgagatatatacctagctagtagtttgtaaagccatgcatgactgcatgagcttctggagctagctagccagctatctgccggcggctacgccgcaaacgacactggcaccgcgcggtcgtcagggctggcccaggcagcaatattgcaagctgatcctggccattgattggtcggccggcctcgcgcgcaggctcaggtggcttctgccattccggtcgtggcgcgcacgggtctgtgtgacgatgattagggagggccgatggcgggccatatattatgcgaccgatatcatttggccaggtacacgggttaaatttccattcaaactttgcctacttttttattgtattaggctctgattccgccagtgatggatatcagtgtcggctcaaaatccgacataattgctagaattggatctggtttttggggtagtaattgacattgatgaggcctccgctgattcctgttctccgagacgatagcgatagagagat

SEQ ID NO: 35 - DNA, 2417 bp, Zea mays
  source 1..2417: mol_type = genomic DNA
actcaaattaggcatccatggtgcaacccaaatgtgttgtcaaagttaacccataccttagatctagaattgctgacactagatccatatggtgtcccatcttcattggttctaggagtagcatcattgacttatagtgattgtagttgtggctcatcattgctaagaacatcaatcagagtggaagtaatgccaaatagctcttcatgtcgttcctcaaagtcatcttcatcgtcaccacacatcctcaacttatagttgctaggggtaggatcatcgtctatcacaggccctttggtacctcaatagtggctactgcacaacaagatgcatgtgacttgcaagaagaagacattcttacatgtttcataccaaggggatacatatgtagtcagggttaggtcatatattacttgcacaaatacaacaaatgtttatgaccaagatccatggatctatgtaaagagaataaactaggtttttaggaatagctataaaaaacagaaggagggaggaaaacatggaggcgtgcagaggggagaagactatagatggcccaaataggaggcttcttaatgccttagtgaccgtgactagatcttctgaggcagcaactagagacaaacatagaggaaatatgtgtgtaggaaaatgaaggtggagctatggaggtaagaggcatctttggtggtcatggttgtagatctagcgatgaggtagctggagacatggagagtggagcgaggagatgaggatgagacatcgatggaggcaagcatctatggctacagggtgatccatgctagggttatacaaggtggggaaggggactgagtgaggggaggcgaggatggtatatgttcaagctaatatgtggggacaagggagtgacccaagcatgaccccgtagtcatggtaggcgagtatgggcccaacaggcctgtcgagctaaaaccctaggccatgggttgtggggattttgaccatctataggtgatttgtcaatgttttgtccatttaataaaggtctattttacaaggtttttatgattccactttcacaatattttaaaaacctacaaaagaatccatgtcggcaacacaaacgaccctagtttactaaacattttcccaatcaaaacaaatcctactagagaaatgcatgtttcttagaagaaatttgaacataaataatattcaagaaacaatacaatattcctaccaaattgacccgacatcatccctcttgaatatgctttttattattatgttgttatgaaaaagattttgcagatagtttgaaagacaatgatatatttgcttaaacgatggtttgagaaatgatgagttgtttgggtgcaacaaatctagtggtagtagtttggaagacaatgatatattgctaacactagtgtttttggagttctaaagatgacttaggcagggttttaggatttggattggaacataaaaaatcatcaaatttaaccaactttggtgaggctctatttaatatcaaacaagtaaaaagttttagtctaatttgattatttttctcacaaaagtgtaagaagcctacatgttcctccttcatcatataagtgtaaacctagaaatccataaccattggttatgaactctaagtcaaaatttgaagacattttaactagatttcattaaaacatgcctttttaacatgaacacctagaatttccatgatttatcagttttaatgggccaagtccgtatgttccctcatcttgatcatggacttgtattttttggtaaaaaatagtagctaaaatagctgatgtcaactttcacacaagagttaaggggggctaaatgatcatagttaactcttgatacagttaggacacctatgctttataatcctagataccactttggccaaaaaagatactgaagctcccttttaagattaaagtattcatttggtatcttaaagatagaagtgtgttgttgtgactaaaatagacatttgatcacctcttttttctaaaactaagatatttcgaagcttgttcatattgggtttaccgcatctagatccattgtccatatgcttggaagttgtgtggtgggcattgagagggttggaaaaaaccttttttgaggcttcttgctattatttggataatttggtgttacacataataattctgttgtttatgcttgagaagacacaaaatgcttcatttatgaatgctacacccttgtgaacatattggttgtgctttatatactctattgtggtatgaggacaaaggggagcagagtgtgaggacatagggagaaggttcaagtggtaaaaaaagtgattgtagactattgctttgagcgccttcaactaaaatgatgtcatgaactaacaaaactttggatttgattttctt

SEQ ID NO: 36 - DNA, 1589 bp, Zea mays
  source 1..1589: mol_type = genomic DNA
catgattgcttggatttattttcgttggcaaaataaattatattcctatggctttcaaacggtgtccaggaattccacctgagagaagagggtgccatgcgctgtgcatggaaagaaattctctctctgtttttttttcaatccaacaaaggagagtcctagcctaggcgtgtcgttattggtttctcacgtgatgtcgtcctccctgtagcaagcgtttaagcgtcaacgattaccgcgtcgctatccgtcgccgttgcgatgcgtggaactgtggaaggcccggaaaactggaatatgccaagaaaaaaaaactgtcaggatcgtcaggccccactgaacctcggtctccgtcgcgaggaaactgccgagaaggtgtaagtaaacgcttgtgatatcctctgaacgttgttggatgatcaggtcgcagccacgtgaggatttacgaaaattctcgcgcttttctttatctactagtactactatttcctcaggtcgaaaacgacgcccatggttgctgttcgggttttttttttcttctgcttctggcattttgttctcgttgtcgcgatttgatatggctggagttggtgtcaggacggacggggcaggttcattgcacccacgccacacccggccctggttcgtcgttttttttgaagcggaggcatccgccatctgatgatcgaatcgaacgaggctaccggatcacgtctgaatgcgatggcctacaaaatgagcaacggcgatggagcggattcgaggtatgaaagggccttgttgtgctagatggctggggctgcacccgcacggccggctctgtgctgccagcctgccactaccggtatgcaaatgcaacgacaaccaccagatcctcatgggggaaaaatacgagacgacaacgtcatagagaaacactgtagacgtcttctttctctctctctctctctcttggaaaatagtcggcaagagcactgttatgtactcctagtactgtaggacatgggaaaaaagaaagctctacgctctccacagtagattaccggcgcgcgcagttactgaactcctcgagcagcacgtgcgagcagtggaatgttcggggcaggaggaagcagaagcacaccacgaggtttcctctgtcagtcgtgttgccaccaccagatttcaattcgacttctcggcgtggtgcttcatgtacacgacaatggaaccaatatatatatttttttatttttttggggtcttgatccggtcggttgcggcgtcagagagccgcgctcctgcaatttcagaatggacagtgtctgtaacgccagattccctccttgacccatgaggaatggatcaaaccaatttagaaacaaaaaaaaatgcaggtacatatgcgttgtgtgtcgacggcatgcgtggtgaaatgaaccacgtgcaggataacgaggacctgaagctagtagcgatcggctatcatcagcacgagtcaaatatacacgaatggcgtggcgtgccttttctgctctctaaagtttcgaacgcagcaggatcatcaagactcaaaagggcgcgaagcagttagttgttgtgggactcga

SEQ ID NO: 37 - DNA, 2299 bp, Zea mays
  source 1..2299: mol_type = genomic DNA
taaagggagacgggacaaacgagtgtgatccagatcgtgacagcaaaatctcgcactggtgaagcgtctgtttcagaagctaccacaccggtcaaaagtaaaatcataagctcttccttgatccttccgtggatagttcaggtaaattctaaaataaaagcggcttgtatgtgttctactgttcttatccacgcgttgacagacagacggcgttgtcgtggaaacgttgccctaccatttctggagcggtagcagattccctttttgcctacctccaatcaacatgcattctcgtctacacaaaggtgatatggtcatgatagcatggccatgcctccatctcgttgccatcccttccggcatgagggtggtgcggtggtatgcatgttagctgcagaaaggaggacagtcgttcttctcttgctgtcctgcacaactgaaagatggaaacctgaaatatggtaaagaacagcgcggtaccaacgtggctttcgcgactgcgatatgcacctgtacactgcacccgactggaaccacacagccagtagcaagtaggcatcatggaatgaaaatcaccggacaattttatttttattatttcccgaaaagaaaagggaatcacaggactatatacactgcccccacacagtgtcacaaagtccattccggcaactgttcccaggctgctttctccgacctcctagcctagcgatgctcgtcgtacacacattattccgctgcgctatcttctcccagtgaaggaaaccgagagggatttgtcacaggagcacaggacgatgtactgtgtgttgctgcgatcgcaagcgataacagcgattcatggtttggaaacacatggtggtttgcaaggttatgatttatactattatatattctttgtcagggacggtaatgctgcatcaacacaacagcaacgcgagtggtggactcgcgtctcggttgttcgacggatcgttagagaccgaccaaatcagcgagggagcatgacgagacaagcagatagtttagcacgagtacaggacaagccagctttgtcgttgtaacggtgattacatctacctgtcttgtaggacaggctaggctagagtaggacggatccaattggaagtccctatgcattattgtgtgggccgggccgggatcaagccgataaggtgataagtaccctgttcccaccttctagatggttggcgctcacgagtaaatctaggttttgtctaaagcaaagacggcttttgctttgctttctcatgtgcgtcttcagagtttcaggcgatatgccgcaaagcaagccgaagcgtcctgtctacccgagatatggcttttttcactgtatggcagatgagagattcctagccgataaaagtcagaccacttgctcggtcaaagctttgtccttagcatttgtatgaggctagagagcgacatattctgatcgccatttcgtgggtgcgcgcgctgctcaacaacagtcatgtgcctttttttaaaatctactgtagtcagatccagctgcagctgatgtgacacggtctcggagagccaggccttaacgaagggggcccaacataatatgatcctaggaggcccggcaaatgttgggccggaagaactgtatgccggcgccattccaatttggaaattcgttctttgttctgctgaccccaagtctgaactctgaagtagtttctcaatcagctcgagctgaaaatattattaattcgccaactagcgaggtctgacgaacgaggcgctgagacattctcggattctccaactgctcagaggcgctgagagtctgagatagacacgagtcagttgcttgatcattccgctaaccgctataacaggatatgtgtagctcgcgtcgtaaaatcgcaacactggaactgttcaacggggggacatcggaattaaaaaaaatgtcgtttgcaccaaaaaaaggaacaaaagagttaattcttttctcatgcgtccatggtccagtccatagggctcaacaggcggtgttagttgtgcacttctttcgtggaacattcctttcgtggttaactaggagccaaatagataatgtccgtattgtctgattggcaagttgctgattaagaccgtcagaattcgcagacgtatattctctcgcctcttgctcctattccttttggactttcggttgcagttggaacaaaggacgtccttgtacggtcagactcgcatcacgctacttactaaaatgttacgcgggcatcgggcttgtgtggctccgttctccacgcgcgtc

SEQ ID NO: 38 - DNA, 2603 bp, Zea mays
  source 1..2603: mol_type = genomic DNA
ttgtagtgcatagttgcttaattattcatctttttaaaccagagtctagagataagtaatcttttgtccattgctggatcttctgttcttaaagcttcatggcaggcttggaaattagtgtgtctcatatgtatcttgtagcaggcactctttcaagtggcgacaacagaatggatgatgatgtgattgatgatgaagaagatgctcagagaagaagatcaagatccaaaaagaatgtggtgaaggaagctaataatccagcagtggcctatcaacttcacccacttaaaattatacttcatgtatatgatactgaagattctggtagcaaacgccgtaaactcatcaccctgaggtttgaatacttggcaaagttgaatgttgtttgtgtcggaattgaagattcagagggtctggacagtaatatcttgtccaacctatttccagatgacactggtttagatctgcctcaccaggtatgtaatttaattcgaagcagatggttttgattgctagttacaccatccttatggaagttttggtgtttgtttatgtacctacctgtttttggtttatttcagcctcacaggcctttaatgtgattttagcagggatatgttttgttactacatctgtatctagtatcttcttggggcctctcatttacgtcagctgaaatagtgacttgtcaattttctcagatggctaagatttatgttggggaggttccaaacttcagcgacaaggattcaaggccatacaagtgggcacaacatttaggtggtatcgactttttgcctgaagtgcctccatctgttggggatgattctagcagagcattaaacactgctgatttgtcatctgggcttgctctgtaccgtcagcagaaccgtgcacagactattttgcagaggatccgcttgcggaaagttgcacagatggctcttatgtgagtcactggtctatgttttttgctgtttgggacatcgatgaaaaaaaccggatggcagattagcctgagagtgaccgtgtaatttgactgacgattttgagctaatgatgcttttgcttcattgcacatgtcattctgagttatggcaatactatgaatcatttaccaggcagaaattgtgaaacataccatttatatcttcttcatatgttctctacatattgattcaaagctaattattggcgaccttcattataatgttattcatgactgttatatacatggcctgtttgattggtagcatccctttgaagttttcgagtctctttgattacaggtggcaacttgattatttgacaaagctgaagtggcctcgaatagaacataagaatgcaccatgggcatcacgcaacccactgtgcagtctacatagttggtcattgacaagttatcctgagtcatctcgttctattttgatgctaagtggtgctgcaagcaatgttgacagtgatgtagagagatctgtgacaaactgggaagaaactgagagtatcagggaggatggggagcttccagttgttattcctgctgagaatgagccaaatggttctacaattttgcaacctgaggtgtcagctgagatccggagccattctagaggcttatctcttatatcaaagagtgcaacaccatctaagctaagcatctcacgaagttttggtagaaatgaggacgatcttgatctcttgatgtatagcgacagcgagttggaggaccagccctgcattcttgacgaaactgaaaaagctactagcccgattagagacagattctgggaggagtatgcttccaaggaattcaccatggtcctgagtaaaactatgaagaatggtctaaaagtcatgctggaagccaaggtaaactatgtcatcactatttgttctaagccttcatgcatctctccatgctcttgcttctgacattaagacttatcacaggttaagataagtatggagtatcctcttagacctccccttttcagattacgtttgctctcagaaaagtctgaaattttgaagtggcacaatgatcttcgtgcaatggaagctgaggtttgtgctaacatatcgtgtaacatgttttatttagtgcatttctgagggtagctcttaaagctcactatcatgctgtgcaggtaaatcttcacatccttcgaagcatacctctatcatatgaggattatatattgactcaccaagttatgtgcctggctatgctgtttgacatgcattttgatgaggaaaacgagaaaagaaaagtcacttcagtgatcgatgttggtctttgcaaacctgttagtggaactatgcttactagatcggttaggggtagagacagaaggcaaactatttattggagaggtgctgattgctcttccagttacttgtagagtatgtgcgatcttgattggtcttggttgagttcagcattgactaccctctggagaaatcatttctgggcattggagaactgcatatataacttaagagctgacacagatccccttgtttatcactactgtgtcttgacagcaatagctgggaagatttctctgtgcccatactgactcgagaaactatttacaatgctg

SEQ ID NO: 39 - DNA, 1609 bp, Zea mays
  source 1..1609: mol_type = genomic DNA
cgcactctgcactgcgctgcgcttcatgcatgcatgctggatcgatcgagctcgatcacttggaccaccgcgatgctcgatcatgctctccggcctgcagcggccgcccagcatcgcaacaagaagaagaacctgctttcgcagttaccatgcatattcatttattccaccccatcgaacgcgatccatgatccatgcatggccaccgtctgatctgagcaccaacacagcccccccatgaatacccatgcacccctacctgtaccggatcatcgctccgtggcttcccatattattcttcatttctaaactgtatgtactagcttttactatatacatcaatatataccctggtatatgactacatataatatcaccaccacaccggttttttaatcactagctaatgaaagattttcctaagcacaaaagggtaaaaagaagcttgccatatatatagatcttgacatgtgtgtactctaaaacagcttgagaaaaactaacccttcagcaataaaaaaggctactttaaagaatatgcttatcactcgctgctaggaataggaacaaacgaactaaagggaccggagctagcgatcgagtacacaattccttttcttttttttttacttgattaatttgaaaacccgaaaggcgccttttgggtatgtaggccggcaggagccttggcagcatggaccaaataaatgtgtatgcgctgctcctagctagagagaacatgctatcttcttggcaagtctctgtctctgatctctccatgcgtgatggccttacctcatcccatcgtatttctgcagatcgatcgactagttatattcagcactaggagtactatatatactatatgcattttttttgttacttgcttgtccacatacacaatgatccttgcatactttgctttctaggcggcctccattatattgcatatatcatcatcatcatcctcatgtgtgggtgcacattatattatttctatctccttatgcaacgatgtgtatgtatgtatgtatgtatgatcaattgatctgtccctagctcccgtgacgatatacaaacatttacacggcgagttttggatggatcgatgtgctgtgcaattgcaacaagcacgatctcggatgcatggggatgcatgcagacagagaagctaggccgaatgaaagcaagattgcatacatggatccaagatgcgatttttatccttgttgtttggtgccgcctgtcctagttgccaaggcgagcgtatgtatagcatgtacaactgtgtcaggcagatgcaggcaggcaggcatatgatgcttttatttgcaggcacaggcatgcacaccaccacacccggccggcaccggccagtggagttgcctatgtgatgagaaattttggagctttccttgcggcttcggtatggtccagcttttatcatgccaacgactaacattccctgtcttgctgctcttactgagatgtataggtaggtaggtaacttctccaaaataaaatacacattatgttaattatattgctccatcatgattcatatgattatgagaccactatattattctatactatatgttgatagcta

SEQ ID NO: 40 - DNA, 1794 bp, Zea mays
  source 1..1794: mol_type = genomic DNA
actttttcttgaaatcaattaaggggctcagattttatgtggcctcttaccttctagaagataatgagtagtgtaccatagcaaaggccatgtgatcatactggatcatctaggatagaacatatgtgtcaagtagacattgggctatacacttattttatatactctccgatgatggtattattataacaacaatatactaataaaatatatgctctcctttgattttatttccatgtatatatagcaagattaatttacaggaggagtatcttaaaatgagcatattcaaacaaataaaaatatacactttgtccaatcgtcatggacctgtgactagccacttcacctatatatatatatatgcatataaattacacacagaaaggagttaaacaaattgtcaaaagggagggtgatacggtaaaatcgatgggccaacggaaaggtagagcgtgacgtgcacgacgatggctcgatcgaagcccttccaccaacttgtgatttattgactaattaacgccaagtgtcaagtcaaacccacagcagagaaagagagagagctagagagatctagagagagagagagaggggccaggatggatggatggatggatggatcaatcacgcactaatgagctagcgcaacgcaaagggcgggagagagagagatcgagcagagcattttcttgcatgcaattatatatctcgtccgtggccctctcattttattttcgcgcgcgcggatgcgggggcgggcgggccaggaatcgacgacgacgacgcaactatttcatggtcgggcgctttattttatggtcgaggagatgatgccgctagcttgcactattatttggacaaatcatcgtgcagcttttaattgtcggcacatgaccttttcctccgtgcgatttgtgtgcgtgggcccgccgccgtcgtcgtcgtccctccgcccgatcccatgcatgtctcaagctctctctacaccatgcacgcatgcatgcatgcatgtgtgtatgtatgtatgtatgcatcatgtagcagctagtaggctccattgatacatcgtcaagctttcacaactttaaggaaataagtatgctcaatatatatatactactactatatactactaggacggagatcgacaaggaaattaaaaggagggaagagatcgagagagacagaaagaaagagagatctcgtcgatcgagagatcgtgaattcttaattaatcaattcatcagctaattatcaagcgttggttcctagtcaaatgagaggatcgatcgatgggccactagctagaggtcgctctctctatactatgcctagcgctagttacctatatgatgcatgacgacgaacctctccggctcgtgtggcgttcgtttttgttgatcagtcgctgctggagtttggacgataactaagtgaccacaggagattggccggccgacttattatagttttcagcagcagcagcagatattatatttgtacgtatatatggacatatgtatggcgaatcttatcactttcactattttaatttaactagcatatggcaagcaacttgattttttattttaagcactttcaatctgtagtggtcgatgatacccaccgactctgcattgtttatttgaaaattttttatgataatctaagaccaaaacaacaataagataaaagcgcatgaatacatagctaactttcccatcaaataaaataagcgattggttccaatacaaatttagagacaaaaataagtattttaagattctatattaa

SEQ ID NO: 41 - DNA, 1898 bp, Zea mays
  source 1..1898: mol_type = genomic DNA
aggcgacaacgatcgcaacggattaaggcgctgagttgagtttcgtggccactcatacccctccaaccaccactagaggtcacacatgagcaccaaagtaggaggaggaggtggtaacgacgatggcgatgctagtgacggcaagcatggcagagcgtgacgccgctgagcgttgcatcggcaccactcatgaacaccggcggtggtccgagggtcgctctagaactactcatcaacaccgatggcaatgcacttgaaggcaacgtcacttctgagcctggatctagcaaggacaacaggtttgttctccctattaccacctcaacagtgttattgctgcgatcttacaagccctgcaaggtcattgtggcccaacttcctttagtgaagtttgagtatgctaaacctgttcctaggaagtttatgtatgtgttggcctaaattgctttacttagggattatcttattcaagatttcgtggattttgcagcattttacactaagaaattacatttaaggaatactttaccaatgctaggaggaaaattttgagctaggatggcaataggttattgtgcaaagtgacattaccaaacataaataacgtgagagttaagatgacagatgaaggtttgcttatcgagttgggtgtgcctatttaccaggaatcatgttacgaacaagagttcaaatagtgagttcacattggattcattcgttcttttgtatggacaagctcacatagttaaactttccaatctccgcttgacatccaataatgaatggctaaaataattagtgaaaattgctaatttgattttaagggtcttcattctttacgaaaagtaaccaccctatctgaatcacttaatcaataagttgaataaacctaaattaaaccccgttttgaatcatatagcgtgccaatataatctcactattacctacttacatcgatgggtcagttaaatgtcatatatcaggtgaatatcaaatacgatcattttggaaaattgcagacaaacatgtttagaagtgtgattgaacttaccaaacttgagtcgggttggttaatgaggttacgacgtattgtcatattcagtagcataattgagtgcagagtggagacggctgttgtaataagaataatgacatatgttaacacattaaccggtgcttttattctcaacagaaattataacacacatgttttaaaaacattgtggattccaatggaaatcagttatacagtattagtggaactgaattattggtttacaaaactttagtgactttctagcagatatgtagtttaatcttatggacaatgatttttaaatacatatagatgttaaagatgtagaaaggaactatatcctgtcactcactcaatcacttaagtgctttataaaagagtgaaaaactttgactcatccttcatacaagtacaaattggtgataacatgatttcaaaagttatgttaacaaaaatagaactcatacttttcattagtgatctagacaaaagtaagaatcaaagatgcaatggttagtcaagcgacgattggatttagatacatggataaaagaactcccaacaatatatatgtttatcacatatggtgatgcttgatatgaaactataatccaaccgaactaattaatctgatgtttgatctatacgttttgataaataaaataaaatgttataatataataatatattttgttttatatagtattttattttctcgttaaagtttatatattatttctgtgattgagttatttcggtgacaaaataatatattttgtttatcacataatattttcataaaataaaataatgattgttgtttttttacgtatgcttgagtttgtgtgagatatagtggtat

SEQ ID NO: 42 - DNA, 2594 bp, Zea mays
  source 1..2594: mol_type = genomic DNA
gtgtggccccttcgttcatttgcgcgaaagcggcatgattaggccgaggaatacgaccccggcgagtctttttaaaatcatcggcgggggcacgctggtggatttgcttccgaggcaggcaaggcttgctttcgtcgcgatccacatgctaatggcctccggaaaaggctggcgtcgctcccagagcgaatcataagacaggcacaggcacaggcacctcctggatggtataatacccctagcctttttcctactttatctttttattcatttttttatataaaataactgttgcttttgtttatctttttagttttcactagtatttattatacagtaggacggagtgagtggtattttaaaacggggtgagtgaacctcgtgacattgaccactaccgagagcgggagatgggctgggccgtgggcgtggtggcccccaccagcggcagcaccggcaggggaagcacatgcggcgcctgccacaccgccgggccccgccagcgtggctgctcatcaccgcggcgcccactctgcacctacgccgtggaagcgcggagcagcacgccagcaccgcagccaccacggacacgagccgcggaggagaggacggcgctctgtgcgtgctagtagcatgcagcggtaccactgtgactgatgaacgaggcccacctggcagccacgtacggagcgagggggggcagcattagcattgtgcttgcccgttgttgaaacatggcacgtgcgcacagtagtggcgggaaaagggctctcgccgggccgcggcgcatgtgatggagaacggactccgtctcccgtgtcagagaggccgacgttcatgcggagccaaaacagactgcgaagcgaacgaggcgtggccagtggaatgcttgctttgcttccctccctgcaactgcaagccatccctcgccatcgccaacgccgaacactgtggatctcgaaacagaaatcaagagcggcgcgaggcagcagtacacagtgctgctgctgctgctgcctgcctctgcgtcactggctcccgcaatgaatggaggccgctgcgccgcgctctgccagtgcccaccacgccacccctgcctgaaaaagggccctgccgctgccgctgccgtgcccacgcaaaaccaggcgcgcgcagcagcggcacgcaggcgcaggcgcaccgtatcccatctcgttctcgtctccgcccaacggcccacccatcattcagcaccccctccccttgggcctttggtttggttcgcagggaaaaaaaagaacacacacaaagctcgatagacggacggtcacgtggcgcgcaccctgcctgccgcctcatgcttcgccacagccgtaggatgcggtacgctaataatccacgtacgatgccaaaagcttctcgtcaatgaccgtagcggatcaggcacacctgagtgcgtgcgtacggaacgaccaccttgatctcgaactgccttccgaacagcggccgtgcccgtggtagtgactggctgcctggctggctggctggtgacaagtggcagggcgtgcgtgtcgtcacgggccgggacgggggaaagggaaacgaccacagaaccgagatgccactaccgctagcacgcacgcacgtacgtacgtactactactactgctactttttcgtacgtgcgaaagtttacactattatgtactcgtacaccatggctacgacggtacaggtgacaggtctactagagctgagcagcagctagcgcggcgcgcagccaggaggaggtgctgctgctgcttttgctgtcaccgtcagtgctgctgaggtgtgcgatgcgtggcactgtacagcgattcggttttagagtcgtccggcataaaacagggggcgcgcccaggccagggcacgccggccgtccgcgatcggcggtcgtgtcccgtccgcagcaagggcgaggcgaggcgaggcggcatgagaagtttgcgttgcgtgcccgccggatggatcaccacgctaaccgaatagggatagaatggtgtctaatactgctacggccccggatcatggctcctcactccccggggttcaagagagagcagcagcagcagcagcagcaggcggagccgcggaggaaagaagcattgcagttgcaggcaggccgggttgcagcacaaaagctcttcactgacgaaaactaatgaagcaggaagcatgcaatgcaatgcatgcatgacgcgcccaaaggtacacaattgctgcagcacagcaccagcaacgccaaaaaaaaaagaggctcttgtcgtgacgccgagttgggcccgcacgggcgggtgtgcggccaccagactagagagaggggccgagcctttgcacttgcagcctttccacccacgcggcccctcaccaagcgcccggccagaggatcacgttactgtactaggttcatgagaatatgacattagacaagtacgtgggtaacacgagagctgcccgatgcgggcaacgggcaacgggcaacggcccaggcttttgctcgcagcctcgcaggttttttttttaatagttgcggaggatttgagaggcggtgtcctcgattctctctattcccc

SEQ ID NO: 43 - DNA, 1814 bp, Zea mays
  source 1..1814: mol_type = genomic DNA
gtcaactctataaatcaatttatagggatgactagaaatcattttccataaatcggggaaaaaaacagtactacctataaatcataaatcagaccaaaagcagcgtgtctttaatccggattttttgtgcaaattaactacgcgcgaatgtttcaaggcttgagcgcgtctcttctaatcattccactctaattctccaaggtcacttgtgagttgtgactaatatgtagatatacattccttggagttagtggttcaaagcgagcagaataactacatgcatgtttcagctatgttatatttaccagctaccataataagtagctgctagctagcctgtccatgctcttcagacacttttggaggcagactatgacatcactgaccagctgaagtgttgatgggtggccacttttcctttcaacaaagtatcaggtttccagagttgagctgaaatgatggtgaacgacgcggcgcaacgtctcctttgcccactttatagataagatatttcttttaccattcaagcattcatcaggatgagtgacacttgaactgagcagcaatctgcaaaaaaaacatgagttcaattggatctctctctctctctcctatcgagatggcagctgataaaaaacaagaaaagcaggaagatacttttgtagaaataaagtatttcgattgatgaactaaagaccaagtttaaccaatctctgcatattggttggtaggaaaatcgaatgatatcaaagcaaaaaattcagataagaaagtagacagcaaatcggtagctttttttggttagtgttgtcctaataggcagcatatctgaacctgttttttccaaaatgccactttacctgatgcaaaccgatatgcctgcctgcttgcttgtgggtatcaaacttggtcggcaggatgttcctcagttttcttggccctttgacgctggctaataatagtaccaacaataaatggaccgtatgaattcatcgtccatgttgtggtggtataggattctcaaaattgtttcagattctgacattttcttgtaatggcaaaatgtagcaatgtactactttacacagctaaggaagtgaccaatatatatatttttaatttctttgtctcatcctaaaatctcctgtcttacaagctctagtgcatataggcatacaatactgaaaagccactacacacaagaatctgttccttgtgaaagattgtaccctactctttctcatagttggatgcacacaccaattgcatttcattagattctattaattagatggtgtactattaagaaccttcgaccagtgtatatcatgtactgcatctattcttccagaaccaccatgggcagcttcctgtcaagaacaccattattagttactggtaggtgctagcttcgacattccacgtgagcagagcaagtagttctccatgctgcatttgttccctgccggagatgctaacgagatcacaagatcaggtgtaatatactgagtataaacgatttatggttctcagaattttttcagtgctatttgaagataacgaatggcgtagtctgtttgatgtgtcaacagttcatcctatatacaatttcccagttgcatggacatgataaatgagtaataatgccagggccataatatgtcatagaataaactctttttcccctttaatgactaaaagtctatattgtgcacacatttcatttgactgctgattgctgatcatatatataaattcgttgccttttttatttatttagattttcttttatgggaacaactctgtgcataggc

SEQ ID NO: 44 - DNA, 2071 bp, Zea mays
  source 1..2071: mol_type = genomic DNA
gtctatacatgtcctagttcttggctccatgcatgcccttatagttcctggctcatcatatactactgatcgatggactcctggatcgctgaagctgctgcgtgtacgtagttccgtgcagcacgcatgttacgattacgactttgttagcaggtcgttgttatgcgccgtacgccgcgtctaagcatatatagtcagcgtcgtcgtcccccctggcaaattatttggtcgttcttctctgacgatgacgaaacacgttcaaaatgcatgcgcgtgttccgtcgcgcgcgcgttcggtaaacgcgcacgggaacgacgtacgcgagctcgtgtcccctcgatcattggatggtctcgtacgtaagtttactacccgacgttgcaccgcgaaattcaaatgccagtgccgaactaagtttggctgtcatacatcgtcagaaggaaaacagaacccccaaaaaaaaacataatgaggtattcccatcagaggtaagcagcttaggcggctgagttgaatttgactgaataatatccatgcttgtgtgtgcgtgcctaacgcattgtcgtcgtgttctacgtacgctgctgcatgctactactttgctactaaaagtcatgcatcgtctcaggtagagctaggctctaataatgtactagtttatttgaaacgtacgtccggcatacggaacaagtagtaataatgagcatcagatcgcgtgcaatgcagaggatagcagaatatacttgtatgtagctatgtataggccacgacttgcgtaaagctctaacggctgttcacgaccccaacgtcgtacgagaggccgggaaacaatcgtccagcaggccggcggccggctagctcggttgccgtaagctacgattcttagcacattaatattacattacatgcatgcatatcccgcaccgctgtagcatctatacacagagtactccacatctacatctcctggaggtcgatcgacctggctagtactagtagtacacaggccgcgggcgcgcgcgtttggtgcacgtcgtacacaacccccttgaggaataatcgcatgcctcgacgacgacgacgtcggagtcagcggcgtggcaacaatgttggttagttgggcaaattaaattgaactcccgaagagagacggttgaactgatgactgagatgagtcgtcgtaaacgtgtgtgccaaggttgggcagggttgggttgggttgggccgggcgtacgtatacgtctctgatgcatcgtccccctaaggacggccgacgacacacatctgtgacagtaactgacaaggactgcatcactacgacgatgcacgcgtcattcggattgtctgcttcagaataagcgagagctccgtgccgtacgtgaccaaatcaaagcacagtcgctaaccccagcccagatgttctagttgactaagtgttgtttactttttttccccccttaagtagatcgagtaacatcgttatcgcgcgtattgacggacagataaccataagctaagtctatataagttcggccggtgctgagctactgcaggtagcaggatcgatatagcccaagcgatcgagagggcattaattgattgtgcttagttttgtcggttgccgctttgctctggataactacaactactccagctagacgtagatcgacctagcgagagctgtggactcgagagcgagatttcaactcctgaacgcgcagtctgtacgtacagctacggcctatctaacatgcctttcacttcattcctcttctggatttcatctccgcaagtcccctgggatttgctcgttttggttcagaggattcgctgcttacacgcacggtcctggggttaacggagctcaacacagtagagtaccatctctcattctctcttaatatcatcggcatgccttacaactaagagtctgtcgtcttgaggcggttgatataaaacattcaccattgttaatagcctattactcctgacgacagaggctatacctcagtctgtagagccatggtgtagcctccatcttggcatcaacaccgagcaccagtattattt

SEQ ID NO: 45 - DNA, 2556 bp, Zea mays
  source 1..2556: mol_type = genomic DNA
caaattaaattaacgctaacaactgattgtaatcagaataaaacgcaccacacgtaatgcaagtcgacatatgactgaaatattctagaatagattcccttttgagacattgtcgtgttgttgcagagtccaccaccaaccatatatttattcagaaatgctgtcaaattaagcaatagcacgacgtgtgaacgatcaacatgactatgatgcaaagatccaaggtgcggtgcgcgcatcttcctcctcaaactggaactcgacacacctgccaactgagatcttattagttgaactttgagcccctgagacgactcatttggctccacaaggacaatatcgctgctagcttgttaacaacaagataagaagatcagaccagtgtctggaaagatgcaaatgttcagtgactgtgtcgatgaatgattttctagatgtgaataataaaacaatttaacctaatcatatcataacaatattaacaaccgacatgtaggcccagcgccatcatgcatggaccatcggccatgttgaagtgttgaagcaatttcattggttaaagtaagcttataggtcaaccacagcaacatttcaacggtttgtagtaggattttttttttttggcaaaggtgtgtggtgcagaatttttgtgccagtacccttgacgccaccgcatgcaggtgccatgtgtcggaacgtggctgcgaaacaaacataccccaataatccccaaattttcaccaaaatctttcaataaatagcgcctttcttcttactttgataagagaaatgatccaaatccaccctcgaaattcaacacttttgccaatttcacatgtactctccctcgttttagtcaagcaaattatgatacccaacctgcaattagctctagcttgcttgctgtcggaacatgtcaaagatccgacaaaagtatataaaatgttctatgttgacatagtctatggccttcagctgtgttcttggctacaagcatacgctcgagagattaattattccctgctttggtttgtatatagtttcatttcttctagaagcctttgtttgttgcatctaagacgaagaaccaacacgtggcctctgtagttatttagggtgaggcaataaaaaaaaatttatgcttcgaaccttcttctgtagcctccctgctgcatgcctattcgtatacaaattaacatgatgccaaagtctgcataaggttttccttcaaaaagaaaaaaaaaacaagtccacgtaagattaacagtgacagattggatatattcagacacatttacaggtgtctttggtttagttgccaatgacatgagggtggtggagaaaacaatccccgtgatcatactaccaacccctctttttcctcacaataattgactttcaccgtacccagtttcggctaaaaaataatatttttgaaatgccagccaaacatcaaacagtaaaattcacccaccaacctaattgtgcttaaacaaatgacctactaactcatagctagacactacaaaaggatcatgctgtcgctgttggcggtatattctccagaaggggttggacgtctaagcatctcacatactagaccagctaggctgtaatcaccgagaggaggaggggtttatttctttaaaccatgcatgccaagaactgaagggatggaatggaaagtgcatgcatccagtcaattaatgaggcacgctaccttatccctgctacagagatttgtttaaccgtctcgatctgtgcaggttaattggtctgcctctgaatattcagaatcatgcaacgacgatgcgtatacagacacacatcatcatgcatgcacaaagaagctagacagcattaaactagtgcaataatgattccagcatattttaactgcattattgtggatccagtaccgaccttccccaggtgctgctgtggcgagctactatacggtgggtttgacactaattaattggatcgagctacgttactcgatcaatacgcatcgtcgtgtttgtggaggggttaaataaaataaatacgtacggcagtgccatgattggccgttttatatataatctctcccagtcgtaacatgcacgctggctggctctacctctaccttgcttgtctgactgaattttagcatgtgttgtggtcaagtagatgatgcaagtaattagtagactgtaaagcttagcatacaatgtcgtcaggtggttagtgtgagagcctcttacacgccactgaaagtgccaaacaaaccttcggaacctcggccttttcgtttggctaaattaaacaaatgtgtggccttcattagattggatcgtactacgtgggcgataaaggagacaggtagcggaaaccaacgacgcctttgggtaaaaatataaacgaactgccccgacccttgtttctatcatcgtcttataagtatatacatgactttggttctttaatttggggggtccaaatcaactggttgttgctacatctactactaactcgacgatcaatgcaatggaattcagtttaaactggcaagcg

SEQ ID NO: 46 - DNA, 1577 bp, Zea mays
  source 1..1577: mol_type = genomic DNA
aaggaccaacaacaagccaaaggaactagatcactgacctcccattgcactgaggagggctttgcgaagggtgctcatgcacatcactctatatctaggaatgagatgaacacatgaatgtttcaatcaaatgaaaaaggatggagagtaataggatggcaaggagtattttttgaaatttctaggcgttttcgaaataatcataaatgttggtggttgtgatgccttttatgtaaccgcgtagaagaagagagagagatagctatgcttatgataaagaagatgtgtgttattgtttgtatatgataatttttatttaccatgtgggtattgatagtgatgcctacatgatgaatcattttgaggagcaatgcttgttttcattattggccccaatgttttccaagattggacattatggaaggccttgacctcccaccggtttattaggtaggtctcctttatcttgcactaagacttctcatgttcatatccttaggcattggacatatggaccatacaaaggtgaaaggaccatcaagatcctatttaggtccattaaatatatatgtagttagaagtagaagtggcaagtcaagaggactggtatgtcatacgtcacatgggtgcatgtcacatcggagtgagctagcgctatcaagaagaaaataaaggtaaatgtagcaatgggtatattagtctactatttgtacatgtgatcctgaaagagacattaatatgttggcctagattggacttcggtcttataggacatgataagtaataactgaaactactttcccgttccgtttatcttatttttcttcttcgcttcttccatctctatccctatccctatccctctctctctcttttaaattcttccccaaatatacatatatatccctattgcatccctggatcgaaagggacatgacaattcgtatgagatctaggctcttcatgcaggtaattcctttattaccctcttggtcttgagtgacaatcatcattaactagtgttttcattagacttgcacttccattccaggtctgtaatctagtcattttggactagagcaattcaccatcacttgtaattggtaagcaacttattctttttaatttgcactatcatggaggatgtggaagcgtgattgtgcaaattatttggggacgaacactcataaagaattttggcggagttgatatgctcaagtaacaaatcatcaacatcgacatcaattgtatcgactaggccatttggatcaaccaagtgcaagcaaagccggacttgtcaatgtcttttctaggaaattctcagcaagaacaacttcatgtcatgcatgatcttaaggagtcccttggtccaagggtaggacctctagataaccatggtccacaattgatgggaccgcctcctcaatttcaacctctaccatctcattctcatatcatgcaggtgaatttacttgcctcttcttaaacaagtgatttgggtttttttattatgcggctgctgtatgcattgtgatgcttgttgggtttactaatgcaagctacggtgggcttgtctt

SEQ ID NO: 47 - DNA, 2006 bp, Zea mays
  source 1..2006: mol_type = genomic DNA
agagaagtgcacaaacgaccagctctccagttcctatttgcaggaagaatggtctgaacagaacctctacaaaacctgcaatcaattgaaacagaatcaagaacccatcctttctttccgaaaagacactttagaacagcaaatgactttaaaagtttgccaatttgtatcctggacttccaatgtccacaatatcaatacttccacagccaaacttattgcagcttataacgacagttaatcacttgaacatctgatagaccactgatctcaataaagttgttcatcgatacagtgaattttttgtaagttgtcaaaagaactcaggttatagcattttccagacaaaattctagccatactcgtaaggggaggggggagggggatcagccagttggaattgatctttcttcaaacctcaacaggcagaaaactaccaagtgaatgtttagattttaaattacacagatcgaagggagcaatctgaaaattgcatggccaagtagtgtatgatgaagttctgtcctagaaggtttctgtgttcgcagagaggaatgggcaaatggctagaactctaagctacattttaaaagatataaagttctcagtattcacacatacatttttcaacagaaactgtggataaatgctagagtaaaatagtatgaccaaatcgtgatccaaaaaacagaagttccaacgaggcatacattttgcaaccatcagaagtatatacttccttcagattccgtatcctctgatcaaatgcaacagactttcccgtttcatcatctcgaactaggacctgtctctctgcatggccctgaaatagtgattttttagcaacatggtcaagaactaatattaaaacagaagcatctgaaccaaaagaaaggaacagttattggcatctataaataaagaaattagcgaagtgaaccttgcgcattacctgatccactaacacatttaatatagtaggaatttccgaatgaataactgaaagtactgttgggtgtggcgttgacggaagcaaatgatctggaatgtcaaccctattcaaagatggaataagaattcagacaaccaaatatgaacaaaatagaagaccttattgtcttaccgtggtttagtgaagtcatatataataatttgaaggttgcgatagttcacttctttcgcacattctcgtaaaacgagtgaatccttatgacatgagacaatgaaagcatctaataacctccctaatgcatattcaattgcaacagaccaggaatcgcttgccagttgctgcatcaaagcaaataacgcaatgagtggaacctggagtgaacaagtaaagtaatcaaaagaaatacgacaacaatcaaggatagttatgccataccagatgatatcctattggaccaataggagggcatttgaatttactcatgtgtctctctactgatttatagaggctcggaactctatctcctccgaaggccgttagctgtaaaggagcatcactagttgctacaattagtttatacaaccaaatagttgttacttaaaatgtctgaaaacacagcattaatatagtagaaatatttcatgcttaatgaagcattggatagtactttgttttgttggcgccgtcgtatatcatcaatttgacttctcagttgattgatcctacgtccatcttcttctatctgcaaagtacatacaacagaatatacagtacaacaccacaaggatccaaaacaaaggttaacaccataaacatggcagcgcggacaaaattacatgttgtgcagataattgtgactactcgaattattcccctatgctgaagccatgtcgctaaccagggaggagtatactactgggttttggtcagggctaggggcatctcatttttccttgcccttgggcctcagcagtcagtagaggtcctcttttccccttggttaggtcaaggttgttcgatgttttcaagggcgcgtgtgtaaccaaacacccta

SEQ ID NO: 48 - DNA, 1795 bp, Zea mays
  source 1..1795: mol_type = genomic DNA
ttgtatatatataaaaaaggaggctttgtggctgacaccgccatcccccaaataaaataggcattcgcagctggctgaagagaggggatgtcgcattaaaaaaaagatgaaaatacctctggctaaaaaatgagtgtcggtgtatatataatactaaacaaaaccagattttttttacaatagacttaaatcaaagaacgtggtgtgggacagacaaaatcactacgtacgactacagagtgtgccatagacaaatggctcaaaaaaaaacaaaaaaaaaagaaacaaaaaagaacgagcgttgcgctgtgcaacacccgttccgcttcaatgccctgccgtccttggctcctccttccgcctgttctgttctgctctgctctgtttctctctccctctccctcaggcactgtagcaggggcgtgtggggcgagtccctgtgcgcgcgcctacgacgaacgagaggacactgccacttcgaactgggaattaaatttgcatggggccttttggcccgtgcagatgggaggttggggttgccgttgccttggtggaccaccaccacgccccccgtgcactgtgcgccattcatcaccatatgacctacagtacgtaggagtagggctttactttgtcatcgcaaatccaggctgcactgcactcgacgatcatctgctggcggtttagcaactaacgacgtacgggcgacatcctttgatcaggccgcctttgtgtagcaagcacagatttattaattacgtacgtatacgcaacagcccgacagatgacactgcttcttactaattatttgatgcacacatggtgtaccatgcgaaacgggaaaaaaaggctgatagatgccaacgagttacggacgagtacgtacaatgttgttgttacacaagctattcggctctaggtaacaaactagtacagcgagcagaacatcgcattaaggatggcagtaaaaaccaagcgcacaactttcatttcgtctcttatttattcatcgcagtattagtagtgtactactgaactagctagtgttcttcgtaattaatattttatttgacaatttaatcttagatcagctgtgtgtgcatcaagacacggggaccacgccgaaacagttcccggtcgagaagcccctgtcgccatgccatgcaacggtcgtgcgtgcgagaggcgaatcagtggacgacggccgtagtgtcagcaagagtgagcaagcccctggcgccggcgtagccagcggcgcaggctaaagccgcagggcagggacgagacgactagtacgtactagcaacgaagcgagcgaacgcgcgccccaaagtgcaaagcggccaggccaggcatggtgcgttgggtacgcagggtgtaattgcgagtgctactccgcctaggactactctactctactactactcgtcacggctgtactagcagtacactgtagcacacctggattacgggctacccagggctggacagggggcaggcagctgtatactaggtgtttaacggaacggaaaccgagcgatgattgccttgattgggttcgggggcctttgaccacctgccacttttctgacttctgtctctctctctcccccatagaggaaccgaggagggagagagggacgcgtttgtgtcgcccgcggcccagtagcccgttggacggatgggctggtttgggagggctgacaccgacgttacgtaagtccaattgtctggacctggggcctgggttaggtttcgacgacaagattattcagcttttgttttgttaagtatc

SEQ ID NO: 49 - DNA, 3690 bp, Zea mays
  source 1..3690: mol_type = genomic DNA
ggagtagttcttgatattgtattgtgatggtgttaaagctatatacaatcatgaagggtgtaattattgtgaagacattcactttgacgctggaagggggtgaaaaaacatgagcttttatacgtccttctttctcgcgtcttttaatggaaaagattcagactgtagatctatgcgacgatccgatgatgccgttactccaccacgcacagcagtcggcagagcagaggcaggcagtattgcagcccagcacacacgcaccgctcgttgcgcaggcgtgtggcgtccaatcacccccgtagtagtgggccgtaacactctccgccacaggacaggctcattaaccccggtcacactgcgcttgcacagcacgggcacgggcacgggacagcgcgcggctcgcatcgcatcgcgtcaatcgtttcgacgggccgggcgggcgacgcccacacgccacgcccacgccaacgagagccggcagcgtgcgtgctgtcagccgcgataactctccattaactaacccatgcgatggccagctcgcggctccggtgtcaacctgacaaaacccgtggcccggtcggcggcccgcccccgcccccgcccccatcgtccgtcggttttggtcgggcgaccgagccgtgcccggtatgggagtaggcccggccgaggctgcgtgcgagacaagggcgcgctcggcctccggcctctggctccggctgcgcgacctgacgcggataggcagccaaaggcgtgcgtagccgcgtagggctgtttggatttttcatggtagtagcttgtctgtctgaggtagccaggggtgtagatggaagatggtccgtccgatccacaagggatgatgatgatgacgaattgatgagttcaaatgtctagctacctacctacctacctagctagccgggtctgctggagcctgcacattgcatgcagggtcgaggggaaccgtgccgccgcacgtaccgggtgtacattgttgaccaaaacatggcctgcctgcctgcctgccacgtactatcgtgagcttagcgaaacttgttcctcgaatcaatgccagtgcctgtgcctgcactgcgactgcgagggagcgagcgagcagcagtacccccctcgtcacagtacgtgtcgatatgccgtatgcgtgctagtgctaaaaaaacactggcagcgcatataactactgcactgtaacttctttcgtctggaatagttaggtacctactgtgcacagaatctgcatctgcatgcgcgcgtccttgttttttgatgggtaagtataaagcattggtacactggaaacactaatcaatcgatgagcaccggtccagtgatgacatcttgaattttttttcctccctccaaaaggacggataactcctgctcgtttactgtacgtagatgctgtagatacaatggaatcgtggagcatttgatcagacagacggtcctccacacgcgccgccgcggttttgagttgactcctgccccccccggcagaagccacgaggcccatccacgccacggccatcgagtcccgccggcattggcagttggatcgatggcacggcgtcccactcgctcccacctcattcatcagtgtcagcgtgcgtgcccccatccggccaacaccaacaaacaacccaaggcaaaccaaaccaccctccagagtccccagctctgaacctctcccatccatccgccggccagggacaaaggccgcccgaggagccagacagccccacgggcgggcaggctgcgggcgctaggcgatatatatcccactgcgcacccgcacagcctgcctgcccccgcgcgcgcaacaacatccgccgtgcccaggagacgccgaggcaccgctccggcgctccgcctccgctccgcaagtccgcagtccccacaccacacgtggtaggggcgccacaacaactgctccgctggctcgcctcctcctcctcgtgcgcccgttctgccactgccaggccggcgcccgccagggacaggacagcatgtggtcagtgtacagaattcacttctccgtgccttctcctctcctccggggctacctgggcactggaccagaacattctgaagcccagcctgtcctggcccaggcctccaacggaaccggacccggagcggcctacttggcctagcgttaatttgtttttttttgtttgggctccggtaaatcggataagaaaaattaccatcctggatttaaaaaaaatactatccctgatttcggatactcggtaattaaaattttactactgtagttcgatgtaccccgagttcgcataaaccgaaacttgcatggaattgcagcagtaatttatgtcatggaatttatgtgcgctaccaggtcatgaagttcctctcctgtgcaaaaggaagcaaaccgtacgtacgtacgtgtgtggcgcgtggcaccgctagcgctagcgctagccctgtccggcgtcacatgatgcaaacgaagtggccgtaactacactacagacccctcgatcggcactgttctttccatccccacctccgcgtgccacgccgcacttggcaaaacggaatgctgccctgccggctgccggtacctgtatttactaccgccaccattccaccaacacaggccacagcacagcacagcagcatggcgcgtcgctgtgcactgtaccgctacgcccccctgcggcctgcgtacaccaccatcgagggcatgccacgccgagaagcggtgctctgctagtaaggaacgggcatctgatccgacggccgggcggggcggtgtgcccggtacccgtgtcgccgcatgcacgtagagcgcttgcgcttccttccaaccagcaaccaccgcaccatcagggagtaaatgcccatcacccgcgctctcacctctttcacggtcgcggaccggaggcctacccgtatcggcgtactagtgcgtctcacaaaggtgtccgggcgcgcccgcaggcctagagcgagggcctcgtaacgctgccccgaaaccgaaacgtgggcgtgtatccctccagtccagcgggattgcatcacctgcgccggcgccgcgccatgcatggcatggcagcaccagctgctgctgcccgccatgcatgcatggttgattggcagcttgttttgttcgtgcggtcgtgcctgtgtgtgctgatgtcgtcgtctacgcaatacagtacgacggagctagctaagctaccagtagactagagtactccgctccgctgcacattgacgatttttgattttggatcgaatattcgaatggcattggcatggtgtcgagtacgtagtgtacgtagtacttcttttcttgaggataggctgctctggctctctcccgaataagctcgtgtatggatggcagcagggcacccccatcgatcacctgcggtgcggaggtacgggcggggctgactttttccgcagaacgaaccggcaccaagatttattacagaatttacagcaagtcaacactttcactgtgatcgcactgtccgtgaattaacgcggcagctgtacgagcgttcctttccgtttccgtttccgtttcggctgcacgcggcactgtgacatgcgtattcaaggtctgctagctgtcgtcgtccccttccgtttcaaattgcaaatctagtctatatatacaagtcagacggaatatat

SEQ ID NO: 50 - DNA, 1742 bp, Zea mays
  source 1..1742: mol_type = genomic DNA
ccgaactgagagagtaattaatagcatcacagtgtaatatacgagtataaatgaaaactgcgcgtgggcagatctaatcatctaatacgcttaggcagaaggtggttaaacagaagcatgatgtgcatgttaccgaccctgctgcgcagaatgggccttcgagatttaaggaaatcccccatgtaatcaatccatcatcaaactgaggattttggagccttgtgaaaccagcatctacccaacagcaaggaagcaagcaggctagctagggagtagcagtagctagcagtagcccttctggttgctggtgcagcatcagcgtccacagtagtccaggttgggtctggcaacatggcattatatgtcggccgtgaaacattcttggggtgttaatgtgttatatcccaggcacaagcacaacacacgaacgaggttgccgcgtactacgtacaacctggaaaatggcttgccgttgtggacacgcgtagtaataacgcggagcaagctcttgtaatggacagctgtaaactgtaatggaactgttcaaattgcactacaaatggaacccggccgaggtcgtccaggcaggatgcctggaacagatagatacatgcagagtacgtaagtgagtactaaacaaatctgaacaaaggaaagctttcctgaaagtcgccaggcgcaggcgaagtactgatgaatggcgaacacgcaagtgcttgctcccacagacccacactcccagctgatcggcagccatcagctcagaggctcaggcgttctctaaaaaaaatctgccaactatatgggaatccaaggcctcgctgcattggagagcatggatctcatctgtgacaccgacaccgaccgactggcagccgcgcgctggtgttttgatgtcgaacgcggcaaggccggtgcatttgctcgcgtcgtcgtatcgtacgcccccggcggggccatacgcccatacctgccgcgcattaaaaatcctccacgtacgtggtcggccgacgggccatgcgcctgccactccacgccgcggcacggcgtggcactggcaatgttagcacgacaagggcgccactccgcgctggcacacgcgacgtgctggccgccggcccgcggcgcgctgccgcacgcggcttcacgttgcttggtctgccgctgctggcgctcgctcgacgacaccgggcgggcgggcgggcggcacggcaggactccggcacccctctggctctactatccgttcggctttatccacctgcgacggccctacgttcgtctacgtgccgtggagagcacagggcgccacggcgccgtgtttcttcggtgcttgtctggcccgtttggcaacggtggttccttaaaatgcagatccgctcgctgaagagtgaagaccgaccgactcggggatgcgtgagagagaaaatggtgatgaggccaagatcgagtcaatatacacgacaagatggaaggtacgattggatcccgcccgtcgacactcaagccagactacattgatagctagctgatgcctgacgtgtattttggttgcagtgacaggatgaggccgatttaggattttgttttagcgtaggtatgccatagtaattaataatttatccaccaaaatctggtatactctttgatatattataactcggtgtagtctatataatgtggtaagcaattttaaaacaggtcaaaacaggtaaaaattatagatcaagtagcattcatag

SEQ ID NO: 51 - DNA, 1636 bp, Zea mays
  source 1..1636: mol_type = genomic DNA
tgggaatgagggctaaagctagctaatagaagaaacaaaaaacgtgttatccaaaggcttcagtaacaagtctcgtgatatccaaagtaatggatatcgatcgtgctctcgttgcacgattgtgcaattccaactctgaccatgaatcagaattcagaaagctcggaactgaaacccgacggacgaattctcggtgttttcgtataatccgtgcaagaatctgactggttgagcagtgcaggtcctgcacgatgtggtgccctagcttcgcgtgccagaaaagcaggaccagcccgccgccccagtcaccgggcgggcccaccctcgaccgcagccgcgctttcaactgttcaagtaccgacccaaccgtcgccggtacgtcgtccgtccaggctacccagtagccctcatcggcgggcggcggccagcaccaccgccacggcccccagacccgaacgtcgccattcaatgcggcgcacctacaatttcgcaacgaccggccgggtacgtacgcgaccccgcaggcccctgcagtggtgccgctctgcagccctcgttggctgtctgccggtcgccgtcgtcgggagcgcttgggatggtgggggggccaaaccggcggggtagccgcccaggctaggtcgaccagagccggcccatgtggcccttcgcttggtcgggcagcaacgctacgctgtgtggcctgtggctgcctgtctgccgtccccggcttgggttgcaccgagcagcagagctgcagcagagacgtgacgtcgagcctgcaatttgcgcatcaggacacagccaccggctgggcacgtgaacgcaacgcacagctcaaccgagagggtcgtggcgatcacgtgactgctgcacccaaattggctgcggcgtgttgtgtttaagcttccagctggatctgaaatcgatgtgaatgatttgccgcgaactgtaatgaaccgtatttaactcttgtgtagctcgcggggatgatgggttgcagcacaggtgttgggccgaaatcctatggcccatcgctggtgaaacagggactgctcactggcttccacacacacacacacagaggaatgttcggtgcgaattgtgatcctggacctggagggagggagagggggtgcaaatatcctgactcctgagagcgtaataaattttggtcagatcaacgacgttcttcagaaacagaggtgcatcatggcgaaagaaaaatgctggcgcgacgttcatggcaggtagacacgacacacaaaggattactctatcttgtgaaagatctcttgtatactgtcatcacatttactgctgtagatagagctatgtgtaatggatgttcagacaggcgtgaaggactctgcaacttcctttgtcattggctcgttgattcttctcaataatggaattgagatccctgggccctggggtggtaagacaacgtgataatggagtgcatgtatcctagcctcctagtagggagatcgagaccggaataaagactattattcctctgaatagctgtacagctatatacgtataatctgcaaagctatatcttaatcttgttctgatggcctacattgcgtcattatctgctttgctttggtcttcccctcccctcaatgca

SEQ ID NO: 52 - DNA, 2937 bp, Zea mays
  source 1..2937: mol_type = genomic DNA
cagagccgagcacatcgtagtagataaaaggaaggaacacgcggtcctttttctagaagtttcggaacccaattgcacgagtcagcagctcatgttaatgttagcttttttttccaagccgaaccgagagcttgcaagccgccagccacaccaagtcgaagtggagtttcagcccactttcgtcgggaacggaggccgcggcccaatggatccgacagggtgatctgagctcctgtcggcgggccgctgggagcagagttcctttgcgacaagcccacaaggaactcaggtccggagtctgaaacagtggcccaacgaaccggattttccgtagcgtagacaactgttccacggaaacgtaggcgtggacctccgtgggctggtggtagccgacacgaggaatagtaggacacctgcgtgactgaagaggaacagccacggttggtggtgttcgagtttgcgtcattctgaagcctgaacagctagcatcggctttgactgctctcgcaacagcaagtcagcgacccagcttattacctacgtctacatgccaattgacatgacttctcatggctaccctttttatgactgcgccttaagactagtaaaactcacgaaagggggagactgtgaaacgatccagttcggcccgtccccgaccgctcccgctcccgctcctgcatgtggtgccaaaagggtcaatggagctagctagatactcgtgagaactgattatgtctcctaatcatttaatcaatcatgagattgtttatgctcgtggttatgattaggccaagagacgaagacaagcggcgcacactcaagtgatgccccccagagccttcttgccatgcactactactactacggcgccaccgaacaggctgtccccgctttgcacgtccaggaaatcaaaactccggtatagaagtagaaggacggcagctgcagcgcatgtaatggccgaccagggcggcagtagaaaaaaaaaggtcgaggaattcggttcctttcctccaggcagcccttgggctagcatcgttggcaactcgctatcctgctctaggacctcagtcagtgcaaatcttccactagccatttgttaatggcgaccagcgctagtagctagctagctatctactcgacacagtgagcgtcgcccgagcgagaactcaacgcgccgaggcaccaggggcccggggtcctgttcctgtagcctagtccacacaagctgcaggtgcgtgacacgcacaaaccacgccacgcttaattaaccacgtcattccacgatctgttcgtttggcgcaatgattgtctcagcaacggaacaaacaagggttcggctcatcatcagggggggtaatgcatggaggcgaccacctaacgagaggtcttacccttgaccacaccggccggccggacgggggcctgatcctggctaaaacgtgctaacaacggttctcataccaactgttggtcgtgtggatgtacataaccaactgttgcagcacacgtgcagcaaggatttctagacacgtctatatagtacgaaaggagacacgtatggacgaagcagttttatagaagaagaagaaaaaaaaacccaagcgattctgccatcataattgtgccgctgcagtcgcgccttaatagaatacgctaatggagacgaaaagcctggttgttgtaggttgagatgtcaggacgcggctatagagggagctgaggagaactttagtacagttcggtttcttggttcctaatgaacaatgatatgcatgcatgcgtagctaataaggggggtgccccagctgcccttgaattcaggctggggggcgggctccacggcgtctgcataccacaaccgacaggaacgggataatattattattctctccaccggactcattagcgtttattaggaaccaaacgtcccctcacaagacaacaaaagtagtcgagtagatggcgcttatattaactttaagataaccctacatacgttagggtttttgtttttcttcctttttttaacaaataaagtacacattttatttgcactggttagcaggaactgccccctttcacacgctcttgatttcaggacaattttttgaggcattacgtaccgatgtaatatatcaacacgagatacatagtaataactaatttttagttttaccattatacaaatataatcatcatattcgaacatgttatgccctcaccttaagagctaacaaaaccctcggtgttaatcccaatagccaattgtaatcggttgcaccgtcgggaacataaaaaccacatcggccgccaatcataaaaccttgccaacgaattgtcgagaactcactagaaccctaggcaacaaaaaaaaatgcagttctagtagtatgaacagattgtagataggccatcttcgtgctggagcttctttcccccctgaataattaagatttatctcatgatatattcgcagcttaatacatgtatcatgtatgtacactgttgcttgtcgtctggtggactggtgggcctatagactatagtataagcaggcactctggcatggcagccaaaatcagagagacagtagtctcacaacatagtcccgaatcatttaattatccaggggacccaacaatctgcgagcaggagtgtcgtattgccacaaacaaagtgctcgtggcgtcgtataaaagaaaaataggggacaaaatttcattattcggaactttctctcgcacactatggcagaataaagcgaacactactacagatcctacgaccactggatcagcaaccggcaaagggagtactactgtatgtcaaagtgcaagctccagtctatatctagttgttgcgagtagcaaattttgcaaccgcactcaagggacagagagcgggccatatttctgaaacataaatatgacg

SEQ ID NO: 53 - DNA, 1941 bp, Zea mays
  source 1..1941: mol_type = genomic DNA
gaaggaaaaaaaaacatgccatgcaacatgcatgcatgcgtgtatgtgtgtgtgttgcactgcacgcgctgcctgtgtgtaggcacctttgccttcgctggcaataaaaaagcatacggaaacagtactgttaactagtagatctggagagatcgagtgggtggactggacgagctgtggcatctgcatttggcgtacctatactatacagatccaatccacgggggaaaaaggtatagatcaggagagaacgcactgggctgggctcgaaacgaagtggccaagaattgtccttatttgcagcggcggtgaaaatgaaatcctcttggatcttttgatcgggagaaaagcgggcctgcatctgcatgcgtgacgcacccagccacgtgacagaggatgcctgatccgacggtcgtatctgtccgcggcatctttgcgcaaaaaaaaaaaaaaatcatggagggcagatagcgccggtgtgccagccagccagccagccttgggggaggggggccgtcaggatcaggactcaggatttggaccacggcaaagggcctgggaccctcgcccttgttcaatcatttgcacgagcgaatgctgcgctgcgccctgcaggattcatccgactggaggccgagggtgcatgcacgtcagtcgcattgcacggccatggacacgtacaacgcaagcttgtgactctgtgactgggagcggtgagcatgtgagggtgttagactggaggaggactcgagtgacgacgaatcgttgcagactgctggcttagttgccactgtgtgtgactgtggtaagtacagcaggacatgtggcattggcatgcatggcggtgaattaagaccagcgtagtaatcatgcatgcatggccagttcatctcgtctcgctcgcgttactgcagagtctcgctgtagtatatctgtactgtacccgggccgggcggacggacacacagcagcacagcagatccatcaatgccgttgtactactacacacacagctacgcactcagtgcatgcatggagagactggagagtatagtagtactctacatacacacacacgacacgagggtacatcaacagctcatcgccttcatcaggtcagtcattcgccgagtcaccagcagcctggccgcccttttgcttctgcactgccacaggagtagtactactgtactagctagcaaggccatcgatcgctagctgtacgcgcttgcgcctttctttgtggctggctccgtcgcttttccccccttttccgagaaacgggtcgggcgggttcgggcaggcggacacgacggacatcctgtccacgagaagaatttctggacggggttttttttctctctctctctctctcccaaactcagcggcaaaagtttctcaggtccacggcgggcgccgaggcagacgctttcagtgcgttcagattcagatagatgaacgcccgcggtaccggcttccagacaaacatgcctttttttttttgaacgagtatgatgaggagaaaacaaggcggaattgttccaggaaagacggatgcgtcttgactgcgtttcggtgaaatttgaaaaaggtcgcgaaaaaaagtgatccttcagggtgggcccgccggagcacggcagatggacggaatccacacatgacgctgtgttgtttgactaccatgtactagtagtgtttagttataatgcactctccctgagccctgacttaccacgtcactttaaaaaatgctcagacatatataaacgcgatgcatttaactttctactcctggttgacatggaaagacagacaaagataatagtataatactagcacaccacatagatcagggtcagagaggaataattacgcgcgctcgcgcggtctgggcaacgtctgcgtgcctcgagtttgatttgaccaaaaaaaaaaaagaaa

SEQ ID NO: 54 - DNA, 1560 bp, Zea mays
  source 1..1560: mol_type = genomic DNA
tagtactactgtctactcctacgtacgtacgtacgaccgggccgttttgcacgctctggaactggtagttcgctagctagtacgtgcggagagccggagacgaagcgacggatccgttgatcgagcgcgtcggtcccgatggcgacaggagcgcactagtacgtgctagtcgcgcgcgttgtttcggcacatcttccatcgatcgcccggccggccgttgacgacacgcgcgcactgcacatacgtaccacagcgtcgtcgcgcagccgtccgttttctgcaaccagctatagctgcctagctacctagctagtctaccagcagacagacgccaacctgacccttgacttgcgcgttgtttgcgcgcgcgcggggccgggcacacgctctcgcgtacgtacccgtccacttcctcacgcgagaataaacggcttggccttagcttggcgtggcccggaggagtcgttattattattgccacatgcagctctggagacgagacagcatctgccgtgtgtcttcgcgcggaaggaaagaatccagagcgtggccgcgccgcgcaggcagtaggcgacgagacgtacggaggccgcggccgaaagcgagaggtcgggtcgggtcgtcgccgcggaccgacgacgagctagcgaggttgcccccgtgacgcgtcggcaataacgcccgcgcgcgcgcgtatatatatatatatgcccagatcgacacacggctcgtcgaaataattagccggcaggcgtttgtgggcgcggtgcgcgcgtacgtgcacacgcatgaggtcgagggccgaccgatcgccccggcggcgactgacgacccatcgtcgcgccgtggacgattcgaatttggggatccggacgccgccatgggtcgatctgcatgccggtcaaatgaaaaccgtgtggatggagatgggggatggggatgggttgctccgatccggcgtcgctagctagctactacctgtgctagctagtggtggcgtccacggcgccacccacctccactcaggcgtattcgttgctagtacaagtacgagaaccacttgctacgactcgtcgtcctgctaggtgctagctcgtccatgcatgctagctagacgccactgggtgcatgagtgggtggcgtggcatgcaggaaatatgagctagctagagagcgaaattaggacgacgggttaattagagagatagcagctgctatctatatagttagtagcctattattactagggctaggacgagctgatgagggaccgacagggagagagattctctttgtttgctgctaaccccgtgctcctgctcgtgaaggaaaggacggccgatcaggggagaaagagagagagagagagaaagctgtatgtatgcggtgtaggattattacagtttagcttctctctcttggcaagtagagtaaacaaacaaatcaatccactagatgggacgactggacgagaaagggagagagagaggaacaattcaagggccgccggagtgcgtgactgactgattatatatcggaaatggacgaccgaccggatgg

SEQ ID NO: 55 - DNA, 2113 bp, Zea mays
  source 1..2113: mol_type = genomic DNA
acggtcatgggggcacgtgaaagcgtacgcgtgcgcggcgggcgcagcgtgcgcacgttggaccgagcgcggaacggaaggcccgaacccgaagcggccaaacccgccgcggaaaacgtacgagcggggaaggaggcgtgggcgcccatgctgctggggccgccgttagcctcgccggcgcgcgatcgtaccgcgcggcggcggcttttttccatgggcggtgccgccccgccggcgggccctccgtccctatagtcccgtcgcaagcggccgctggtttggttggtcgcttcgctcggcaggaagcgagcactgttgacgggttggcgcggcgcggggggctcgcttttcttgccgtcgactcgtggctggctgcaccacctgactggcggcgggcccaccgaggcggggaagtgggaggagacaggccggatccgcgaccgcgaccggggcggggagggccccccaccccacgtctgtctgctctgggaagtgaaaccaaaccagcagacgaggacgagacaagccagcagccagctcccgtgttgctgccgccggccgctctctgcccgcagcccgcagcccacagacaggaggggcgtggggggcccgggccggagagagccacctcgcgactcgcgccgtcgccgctcgcaacgactttaactgccacatttatggccctgctataaaacaaactgccgcatttaataggctaccggccaggcctgagagagattcgggcagaatggggaatgcagatgcatcagtggtgcagcagtgctacagtaccagcgtattgcattgcatcgccggaacgcgagcaagattccttcgtcgtgaccgctccaaaacgacccttgccttggcttgcactgtcaggtggaagcattgttcactgtcggatacatacatgagatcatgtgtgcgacagactgtacagcaaacagctagctcacatcacaaaacatgtggccggggcgcacacagactaataagctctcgttaatttagtgtacgccgaccgcgcgcgtcggtctcactttgctgctttgcatcagatcaggtaggtaggtaggttatatatgtcattttgttaagaaccatttgctaattaacaacctgcgccgttttggcgccaaagttttcagacagagttaggtcaaaccaatcctctcttctattttattcatcaacgcgtattaacacaaccaaccacgtatagaacgactcctaaaaagaggctctttcttctgcagttgttttttttttctcataatgtatgagcttcgaaaagttcagcaattgcttgctgcacttggattacacatgttcattggtcgagtacgtgtcgtttcagctggatttgatgctgcgatgtagtaaccagctatagtgcacgcgtgggccctttttatcacccccaaacccacctttaaggaaatcgtaaaggagatacgtacatctctatctagcttcaacgaaaggtgcgtgccaaaaacaatggcagatgactagattttgtgattggatgatatggaaagcaattattaatcatcttttgatatctcgcggtcggattcatttgattgatagagagccggcgttccacttttcatatttttgcccctactagcagacagtggctctgtaacttccaatatggatttgtgggactgaagggttaattattatctgtattctgtataatcggtggctgtatgtacgatataatcattttgaaacataacggtacattatttctcattcctgtccaagaagcttaagtcatgtatggatctaactaacaacaggctaactaacttcttagtctatgatgatccaaatatacaaactaaaatatgagtccgctagttaataggactagtagttgttagtttcatatatcaatccagttgttactctctggtaacatcaatctaacaagagttcagtccatgctgtttgtctcacgcaaatatatgtgttacgttgaacgccattcgatagtggtatattatcaacttatcttgtggttaatatgattagcaaaagaataacaccatttgtcactatattttacttctatagtataagtagtttttaggaatac

SEQ ID NO: 56 - DNA, 1580 bp, Zea mays
  source 1..1580: mol_type = genomic DNA
gttccgtccgcatctgcacaagtgatcgaacggcacaccgcacacgcaaccggccggggccgtgaatagtttcctgaagacgattccccaacgacgacgacgacgacgcgcacagccacgcggcacagtgctcatcaagtacacagtagcacacacagcgtgcgtgcggctgcaattaatcatatatacgggttatttaagggcaggcaaagcaaggcgaggcaagcaggaggccgacggcgaggtcacccacccagcggcggcgtaccagcacacgctacgccagtcgggcagtcgtgtgtttttttatgatagaattttcttcgtagacatatagatgctggtattgattgagatgtggctagagtgatcggccggcatgcatgcgatgcgatgcgttgcgacggaggggagatccttctcctgctatctcgtctcgtctctatactagctccgcgccaagaacgtcgtcacagaacgtacttactaccagcagggggccgggcccgggaggggctctggccggtcgacagatcgttggcaaccaccgccggccactcattgagcgagatgcatgggctagtaggctaatcctatattttttttaacacgcatgcatatgctgatcaatgcgaacagacggactgaactcgacccatctggatgtagcagtagtggtggctgctgtttggctggcatggcatggcatgcgtccggcaggcacgcatgacagcatccatccagtaccttattgtctacatcgaatattttgaaaattttaaattcgctggttttcgtcgactggctatatatagcgcatggaaatgaatttaatttagctgcagatggaaggttgttagaggacgaaggagctcaaatcgagcttttgcatggaataatttatacgttcccagcatatatatgcatacatagacatagagctgaaccggcgcatgtctacctactacatacaaacgaggcagatgaggttgctagtagctcgacgatcatgcccttgttcttctttattccccatttaatagcaaatagagagagagagagattaaaaaaagggaaattgttcagtcaggagtcaaattccttctctatctagaggcaatacacgcagtatatatacacatatctctgtcaccggattattgaaacttttgtttaagtagtatagtagtatatagcaagcaagattttaaaattttgggtgccctcaactatacatacaaaattcaccattcacatggcctgcttgccactccttcggtccttaagagctgctgtgccctgccctgcgcagactgcatgagcgcgcgcaagaatgttcagaaggcaacagaacaggtcgtgcagaaattaaattaaaatagaagaatccatgtgagctaagctaagcatgcagcagccgctccgtcaaatatgtcaatgcatcaaggagagtagagctcccaatcctgctgcttgccacccgtcgtggacgcacacgcatggatccgtgttgcatatggccagctcgatcggagcagcggcaggcgtcatcatccgtgacagcaacggcgcaaccaccg

SEQ ID NO: 57 - DNA, 1967 bp, Zea mays
  source 1..1967: mol_type = genomic DNA
gctggtgcgcgtctcaaaggtgctcgaggacttccgcgcctccaacgccgagggttcgcaccattcgcctccctttattttggctcctgtttttgaacttcggagcggcaaatttggtgcgttcgaacgagtgtagcagtagtgttgatgcaccgctcagtggactttgcgcctggggctagagtttgagcgtatcctttccaattcttgccgcatttggtccttttgcttttctgttctttttttttcaactccattagaacagtgtattttttgtcctgtgatcctgccgtccttagtcctgagtgcatttgtattacaacctgcgtcgtggtttgctttgatgtttctgttgtgtactgatcacaatagtgctgctgtttctcatccacatcgtgcttttatcaacatgtctgttcacatttgcagttacatataacttgcacatcggctctctgatgctaatatcgtcacacaaagtttgaacgttttcttaggtttgacatgtgctaagtggtgcattcagtgtttgtaatgtagctttccagtgatatattttggaatctcccaatcttaggttatagaatgcaatcgtctttccacaaacttataactgtttaccattgggttgcatcttgcaattttagacatgattgttgttagatacctgtacgcctcctagatatatagtaaaattgccttgcaaaagtctttgcatgttttcatggctgtatattagtgggagtaccagcacattgagcacaagcacagacccaattaattgtttgatataatttattgatggtagaattctagttacgcgcataagggtcagtgtagcagtagatcttattttgatggcattctttttctcgtatattggctggccctacttcagttgaagcctcacccatctatattatgtagtaatgcattgagattattgctctcttcctttggtgctgtgagctgaataaactgatacttagactattttaccttgcagtgtacacatttgaacctgatatatccaaacaagagcgagctgcaatccatgagatgtgtaggaaaatgggcatgatatccaaaagttctgggtgagtaagataactaggattcattgagcaaaattgtttcatcgaagagttgtaaggaatctatggttttgcttgcgccatcctaatgttatctttacaaaagttacttgttaaatgtctattctgagttctatttgacttcaggaacggggaacgtcgacgcctttctgtttataaaagaaaacagaagcgggggcctgaattggaacaaggccctagctaccttgggttttctgaagaggctaggcatgttttgcaggatttatttatgcattatcctcctggtgatgctgatttaagtggggatgttgaccagaattctagtgataaggctgcaaacatcaaatggagaacagatagtgcgttttgcaggcccacaatgagtaaacttgatataacgaagaaagttgagatgctcgcctcaaaaataaatggatccgaacagttgagaaaggtctcacattctggcttgataataatatcttcctctctttgcttagttgctgctacttattagttttatttatgagacaatcttattgatagctaaagcctaaaggtttacttttgaacaggtcatggaagatagaacaaaacttcctatttcatccttcaaggatgtcatcacttcaactttggaaaatcaccaggtatagtttttagctccaaaggtttctcacatttgctagaccacatcttcaactcacatggtgcactggtgctagtttttcctctcagatgtgcagggaaactgtttttttttcattaaagaagcatagcataatacaacaatccccaatccccaaggccccaaccaaaaaacagaaaggaaaaactgggtttccatcgtgaattttagaacatgcatac

SEQ ID NO: 58 - DNA, 1606 bp, Zea mays
  source 1..1606: mol_type = genomic DNA
tcgcgttctacgcgaaccacagtctaagcgagagagtttacccggtccatttcgtttatccgttacaaatgttggctgacagctgtataaatccatcagctttcacagtcacagtaaggacgtttacatggtcaagtactggtcaagaatacgaacgccagaacacgtatgaaagaacacccccaatcatatatatgtacatgccgccggctggcgggccttaataattctgtaaaaaggtttaacataattggccgtaaaaaattttcactggcagatagctagggcgtggcgatcgagtgtggtgtgtgtgtcagctggcaagttgcgagtgctcagcgtctactacgatccacgacgcatgtacgtatcccggtggatctatcccatgcaatgccaacgcactggtcgcgctagtacgtgccactttattatttattttgagaccggcccggcccggcccggtcatgcatatgtgtggcgcttgtagaaggctgcttgcctgctctgcttgtcatcatcacacacggttgagacctgtcgacttgagatcaatcaactaacaagctagtgtagtgtagtgttgcgttttgattgagaaaaacgaacctgatgtattcactaaaacggccacacattattgtcgcttcgttgggcaatcatatcggatgtttacggtcgaggtacaatgcatggctatatgtgcacaaaaagaaaagctgatgaaccggccagcagcatcgtcgtccgtatccatgcaatttatcgtactgccagaaacgtgaagacacagcaggacttgcacattctttgactgatttgttttaagccgatgcgtcttcgtaagtagtatctcggttagttgtccttcagtggttgtcaggcgcccaaccaacaggatggcatgcgggagatgcggcgtcacctggttgcttcttcgcctcttctttttgcccaagtttcttggtcactttagctcacttttggactactgcgaaatttacccggccggttctgcgtatacaccacagggtcgtgatactttttttactttcaggtcgcacttgcatcactgcatggttcctccggaagcagatagtcttaaagttacgtttggtaaaggtgtggaacgaccgaacgagtaaactgattggtgcgccgcttgtacttgtgatcttctgaaccagagttcagtccacctaccacggtatgtgtggtgggaatacccattcgcaacaaggggacaggtaacttgtttctgatttatagcattgccgtgtgaaaggttggaaccaaagtatgtcgtgtgtaccagtgtatgtggagaccgtgtcttaggtgaggacaagaaaattgttttaaaagattttccatttgctcaagaataaaatttcacatatccctagtgtgatacagaaataactgaataaggtctaagttggtatgcaaacttgttcccgcaaatccgagactaaaaaaacatgtggtgcccacatgtatcagtcttgtaaaccaagagttcaatatgcctaccacaatatgtatgaaagctactccctatagagattcacttgcctcagatttaccatcactacagcaagagagc

SEQ ID NO: 59 - DNA, 2765 bp, Zea mays
  source 1..2765: mol_type = genomic DNA
tgcatacaattacgctaaatggtgcttcgctgagaatattgtagtacctgcagtttgtcagtagtccccttggagtacataaaggaaaccctccagtcatatgcatgtaagacctttggctcatcctcaatactaagttttaagtacaaatcttgtccggccctgtttgtgaatgttatatacgggcgtacatggataacctgttgttgcataacatgaagtcaactgattgacactttaatatgaagttgtagattaacatgcagctacctttgttggaacagcttgatacgaggatggctttgaacaaagaagaatatgcatgcattttccatcatcatcataggcagaaagatcaactgctccatcctgaatatggaatcagtactatttaattgtagtggtagaaaacaagaaaaagaagaaaagaagatccaaactaaccatatcgccaagaggcgacaattctttcacagatccaaaccgtccaccatgtccaacagatgatagaagtcctaatcccttaaaatttaatccagacgctatagtgtatccctcaaccaattcatcgtgttttatatcatagagttgcttctcacccctttccagatgtgaacgtgaaagaaggcgcctcttctctttccttccagaaatatcaatgagacgcagagtaagtggaggcaatctagaaaaagatatccaatagggaacatatattctgataagccttgccatcaaatagtcactatcactgctttgttcaagcagaatctgtacaaccctacagaagaatgaaagacaaacaggataaaagatgattagtgaaaggttatggcattttatccaccataattttttcatgtttgtgttgttaccttcctgataaagagcttcttagattgataaatttggaaggtgcttgggttggatgtgatataacaacaggctcctaggaccagaaaatcatttcaatagtatgtagaaaaaaaatagaaaagcaaggcatctataaaaggaagtaattttatccatacatgcatcagttcccatcctccatgtggaaccagggataagtacaaagggtttcttggatcaacattgtgaacctttacagcctcacccggacttaaggtgccaagggagcaacttgaatcttcattatcgaggtggctagaactgacagtataatgtgcagcaatgggaagatagtatgtcaaacacaacggggacttaataatgatgcaccaatcatatattggttccatctgtacatctttgcctatttccttgccttctatgctcaagcaaaaccacagtccatgacaagaattcgaggtgccatctacctgtgagcagaatagcaagttttccgattcatacagatctgatacacataaatctaggatctcctcctttctaaactcggtttgactgcgcctctcctggacatcactccaggagtaattttcatgatcatgatgttttgccggccttaactgcaacatatagggcataagaggatgtgagaggcctgatagtgggactggaatagtagaattgggctccagcgttccaatcaggatttgatcctctttaccaaaactgtctccctgttcatctgcccgtgcactttgagaaattcttccttttagacgtaaatcaacaatgaaattggttgtattagtaactgacacaagggaacgtatgctaacgtatctgttgccttccttgactgttgcttcactagcaatcacacaattaccaaacctccaacatgctgcaggtacagcatagttcaacttcatgcttgtccatggcccttcttttgaagggctaatctgaatatatccagtcttgctttcaggactggctctgttcccttgttgtacactagctctaccagaaacaagtaccgcaaatcttatttttccactaggtttaacagtttcctgttcctgcaaagaattgtcaatataattttataaaatattgtaagtatatttcgcagaagcacactttgtgtggactctattttgcaaaagtgactcaatacatgattctccaaatgtcagagttctcaccactgtcttagttgacataaggtctcctaaagccaaatcaaatttgacagagtccggattagaggtaggagggagcttttgcacaacttgctccaatggagcagagtatataccaactggttgacctgttctcaacataaactataaactagagttgggaaaaaacaaaaaaaaaggcatttgttgtgaactaaaatcacgaacctttaccagcatcaagcacaagaagctctagagtatagctgtcctgaaacattgcagtgaaaatgaaatgttacttaaatgaaagcatgcctcttggaaaattcaccattaataacagtagagaacaatttccatggatatctaaaccaaagagagtaaaagtgttccagaatgttttgcgtttacataccacacgttcgactttgaaaaagaacatttcgttccagttcacctttctggtgttctgtgacaagtgttctccaatagcagcacaagtacgggcactctgttgttgaacagatatggaatggttttctgaaaagagccgcacagcagtcatatattcaccagtttctaatccttcatcatccttgacctgggatgggaacctaagtttagaacagccaaaaaagtccttcgcaacaacttacttagcaggcaagttaactgcaatacaaggttcagaattcggtgtaaaac

SEQ ID NO: 60 - DNA, 1725 bp, Zea mays
  source 1..1725: mol_type = genomic DNA
ccttttactttcctccagactaagcgatcggaacctgcttattatgccgcacaatctgctatttttcacttctgattacttgtagtggttcaatgaaaaaagaagagtacttatagtggttgcatgtacgtgatgaccgaggtacacaagtgttctacgtgaagtgttatagatcaagcacttgtgctgattggaattgttattttgtttgaactggccaccatgtcatgtagttctatgattacattgatggaaaataaatgcctttacccttttctgaaaaataaaattaggtatccatttaagaaggaacgggcttgtgtatttccagttgcttgttgccttgttgacttaaggaggtcaagccatgcataactttaatgctgcccctttacattattttttgggtaggttgtggttgaattggtgatatgtgtggtgtggtgtagcgaaaggtgctgcaggagaggtctgattattgtttttgaacttacccccttgttagggatgcaacagttgaagatttctaattgccaaatattcattattgttttgagaaaacttctaatttggtaatctttccttgatctattattattgttttaaaaaatataataattttgttgtaaacgaacatatagtaggcacaagaagctagtgattctctttgtttacttgatcatatagttttcaagattatgttggtacacaagttcaaaaattcacattggccgcgcatagcataattgatcatctacgagtggcaatgtggcataattgaagaaggctaactggctaagacaggcaaatcgacttgtttcactaataaccaataatggaaacttagacacactagaacccaccatttagaaatctagtattcatttattgtcatctaaataaccttctattgaaagccatctcccttagagtttccaaagctttgcccattggatttgccctagatttccaatttagttttggttacgttactaggagaaggagaataggagatgtcgcttgcagagaaagggggttgcggttatgtgtgagaagctcaacaatctagatcagttgtggtcttgtgaaagtaggaggttgtgatcgccaagatcaacatcatgcacatgaagatctattgttgtaaacgagtttcgaaccctaaacctaaccttccatacttcatacttgcattccgttttgccaaacaactagaactcgaccagaattttagtacaatagaaagttgccaatttggtttcttttcctataggtttgtttagatacttgctttatgacctttttcccaccaaaagattgcctaagcattgtgttatttttgaaggaaataattggaacgacttaagcccttaggctattggctgtgcttattatatattttgtttatggctacggggtggaggaccaggattccttaggtggtggtcgtggactcgtggtccgtttcaggccgaggtttgacaccccttcaccctgtagtggaagagaaggattaggcagaagggagaggtaggagaagactattgcacttcttgatcccactaagtacattggcttcgtttacaaggacatatgccaatacaagcaaggggctagtttccctctattagaccctataccttcctagcctctattgctaatcatctagctacttgcctttcgtgcctctacactgatatgcctttcccacataacatattgggc

SEQ ID NO: 61 - DNA, 1584 bp, Zea mays
  source 1..1584: mol_type = genomic DNA
gctctagtaaaattgtatttcctctggctggcgagcggaggacgacggagcagaagcagctggcctcagccgctggacagtgggcctcgactagcagcagataggaaaccgggatagagctgccttccccttccggctccgctcagtcaggcctcagatcggtcgaatccagcaccccctccagatttgcgtcaccaatcttcttcttcttccgccgccgccgccgctcccccacaaggaggttagctgctatccccaaatcgattcatcaatcatccgtgtccttccatttcattccagtcggtcgccgcagcacggaccgagaacagagcatcacgtcacatcaaactaacctaaccagcctcgtccctcgctgcgtatctgctgcactttcatcaacaccagtctttctcctcctggattgcattgcccaggcaagagaacgcacgcacaccgaccggaatagccatgatcttctgatccaatccaagatgggcctcaaggagcagcagctagacgccactgaccaaactcgtgatgccgccaactccctcgcttctgtttctgacgagcaccacgagggaccccgtgtctcaagctgcagcaccgacaaggattctggccttccaagttgccgagtctgccattgcgtggaacccgatctaagaggcgagtccgccctcggattcttgggcatcgtgcccccttcccctcccaggactgacactggggggccaaaggatgatgctgccaccagccccaagggggagatattcgtgtgcgctactgacgtcgaattgcagcagcagcaggaccatcttgtggatctagggtgttgttgcaagaacgagcttgcccttgcgcactatgcctgtgcgttgaagtggttcatcagccatggatccaccgcctgcgagatctgtggaactgttgctgcaaatgtaaggcctgacgatttcaacaaggttctcgcgtccctcaaggattaccaagctctcagggaaagtacatccacatactggtggttgcagcagcatagtggtgttgatccagacgctgttgcagcaatacgaaggcacgagatctcatcctggttcaatcctcacgtgcctatctcccaaggccacattgatcaaccgcatccctcaaccaataattcttctgttcttgagcagcatactagtgttgtggcaaacacaagatggagtttggagagtactggagtttttattgctatctgcctggttgtcattattcttgcatggttggtcgctccacatgttggcaaggtatgctgcaacttctgctaaagagtagttagtactacgtacttgtcctttacaaatcataaagcggagaagcattttctgtgcagaaagctgctgtaatctgtcttcatatgcttcttggaggtctatgcatattgactgtagtaatatccctgagatttgtaagtaggcggtgacatatttcatttttccttttagccagatttttcacctctgactgagtttaaatgtcaaaaaaataaataaatatgcaggttttcccgagaatccagtatgggtctatgcaatattgggc

SEQ ID NO: 62 - DNA, 2420 bp, Zea mays
  source 1..2420: mol_type = genomic DNA
cggttgtacttgtaaatctagctgcttgtacgatagttatatgatgtgtttttttatccatagcgaccaccacgacaatctagacctagctagtgcgactgatttttacatcaatacatcgcaaaagctacgcacttagagagaaaaataaacattggaatttagagggtacagtatttagcacttcgttacagatgtgtgaaaatccggagccgattttgcaatagccgtggatcgctgaaatcagaccttccttgtgctgtctaaaataaataaatgttttgtgttcttcgtcggaacaagaacaagcaccggttgctcggaccgtggagaaaactggttgggctgtgccgagaaacaatgagcgggctgggctgcgtcttgacggcgggccaccaaactgtcccgccgtgcgcgcagggcaacaaccacaacgtcatgtgcggcttgcttaactgggcccgttagacgcgggcttcgtgtggccttggaacacggccgttcgacctggcttcacgtgacgtgaaccagagcggagcggggccatcgatttcggccgcgcgaaacgcgtgcgcgaggcctgcgaaaggccgctggatgaagctccctttgattgaagcccgtgtgggccgcaccgcatggtccggccggcgatcgtgaccgttggagtacgatttattcgatgcgtatgtactcagctcgatccatatacgatatgatagtacgtagacatcttagacgtaagttgtttaaggaactctctctctctctctctctctctctcgggtttctgtgttcatctcaaagttttttcagttcaaaaaccaattcgaaaacaaatcggcttaaaattcaggtaatcaggtcaagcgactttactctggtctgaataacttgagacatccgggttgccatggccgactctagacagcggccataaacacggtggtttctttttcttattgggatagtaggtcactccaaataaaggctattgccatatgctaaggagacggaatttgtgacgccatcgccaccgggttaacgttaatattctactactagagaatctagcttacgtttcggttccggccggccagtagaaaactctctctgaaccgaccggtcagaatcccctgctcggtgctcggttgcttggaccgcacgcacgcacccctatatcgtcagtgcctgtaacagttcttattcggtgattattattataatattattccacgtttgcacacaccgcacatccgcccgtttgtaaaactgtgagttgatcgtcgagacgaaaggtggagctggagtatatggctgggcttcgattgccgctcgcaggtcggtgtcggtcccgaacttttttactcgcgctccatcaccacatagcctggcgatcctatcgtctgcctacggggccgcagcggcgctcctccatctcctgggtctcgttgtagccacatatagagtagtagattgttcgtcctcgcaatgatccgtagtgcacaatgcccagtcgaatagtcgatgaatagcacatacacatatatgcgtgtgtgtggtcttgtcaaggttaactgctgcagagatgagatgccaaagaaaaaacacatattctaattaataaagctttgtgtgccgcgacaagctagctaggctactgtctcgtacgttcacgcggtctaaatcacgggcgcagcacaaattcgatggcagcctggactaaacgaggccgtggccgtcgtcaccattcaccgatccacaggattcacccgggggcaaaaccagcgcacattacctttgcaggacaggagttagaggcgcctttttcctggtccctctctctgctgagcacatgcagcagctagctagctcacgctactagtcactcgcgaagaacgaatccccggccggcgccactagttgtggctagctctcgcgtctttacattcgcagctgcagcgtccatttcacaggcagtatacatgcatgtgatcgagtggaaggaggagaggccaccgctggccgctgcccgctgcttttcacgtacaggcgccggcagtgcaatttggcgacgatgcgaggtgttcgccagtatgtctcgctgaaagggtgaagccggagaaagggggaagaatgtttgctgcggcggatggagataagatcgcatctcgatgggaattagaacggccgccggccgagtgtgtgtgtgtggactgtggatcgaacattccgccgcgcgcctgaaaggactgtcgaagcattgtgacatctgtcagtcgatcgatcgtgtggttaacttaacggatgctaaccctagcttcttttttctcttcagtctagctagctttctatcttgggagacagggacagcatttttctttttgtttttttagtggtacctttaattttgctggtgt

SEQ ID NO: 63 - DNA, 2011 bp, Zea mays
  source 1..2011: mol_type = genomic DNA
gcggctagtggatggatcacacatgtttcgaactatcttcttttccctgtatgaggtacagtagtagcttacggacgaagggatagatatatacataccatgctatgcgcgtctcacttgtgtacctacagctacagatgtgcatctctatcctatctcttcactctggccaccttttcttctagctcggaaggaaaaaaaaaagcatgtattattgcatcacttttttttttgcaagggatacggtgcagcagtactactactgccacgcgaatgttcattcacgccgcgtacgataggcaccgctgcatgtacccaccggcacagtactaacggtttagatgtctactacttattaattcaatcacgcgtctgcgagaaagcaagccgacgggcatcttctgcccgagtctctccgcgtttctgtaactagaattgtcatagtcagggttgccaaacatcagcatcccgaggcagtttctttaattctgcttttttttatatatgtaagtttgcttaccgaatgagctagttctaaacaaactcaaaaacaaaacagggcaactgggggtcccttgacattgcacagatggacctgaccactttgagattcccccggcttctatctccttttccctccccttggatcaaatgaacaaaggagcgcattctctctctctctctctctctctctctctctctctctaaaagattaaaaaaaagcctgcatgtagtgttctttgacaaggacaaggaagcccttttacatcaatacatcattcgtatgttgttgttttctgtgttctttgcgttcctttttttttccctccctccgccttattttttctacttgattgttgccaagatctggagcacctgatctgatcgtgtgcgctggtttactgaaccttgggagggctatacgcttcgtacgggacataccaatttcagagaattcagttatcaggtaggtggttcaatcattcctccttgtggatttgtgggaagtcagagctcgcaagcatcgccaaaacttagagaaaaaatagacctgaacctgagaggatgatgatccgggacgcagctttcgtatcatattcgccgctggtttcctcactgcaaggtgtgtaaagtgtaatcctcaaagtttagacgaagcagtaccaatcataggctcacaacgacactgtaaactgtactgcacagcagagcagctggatgatgaactaacagcttctcgttttttttacgcatcatattttttgtatccactggtctttatcactcaccttctcatttttttatgcatcatattttttgttcccttctccttaattcccatgcggtgaaggagagatgcgaactaacagtttggcgctgcactgttcgaccggctaaacacggggccaatgctctctgtacgtgcagatggataggatagtctttgattcttgtttcaagatgacgtggatagtctataatagctaaatgtttgcctcgactactaacttgccgatatgggcgagggtaactttaaattaaatttttaaagcatttgacttgttaaaaaaaaataaaagcctatattctttgttgatggagggagcaagtggctgaaaagccgttgccatttctgggcgctccctaaactacgcggcaagcaggctattgggagcccttgtcgctgtcgacgcgatgtgcggcctctttcttcgtcctcttggttaggtcttatctacatggtcacgcatccagtttattcattaggtacgctatctgtgtgatcgtatgttcagtttaatttatatgtgttagagtataaaaaaatttatgtaaattttacattagcttgagtcagtcagtcagagcaaattaatttagcggctagaccgctagaggctagtcgcgtgcgtgcgtcgctgatactcaccgtcagtccgtcaccgacacttggcctgggcggcgtgtagcagcagcacggatacgggcaatacggccgggtgcatggtct

SEQ ID NO: 64 - DNA, 1766 bp, Zea mays
  source 1..1766: mol_type = genomic DNA
gctggacttcagcgccataggtattttcgtcaagctcgttgccaaggtttatgacattgaggtcagcgccataggtcatggtgttaccatatcgttacgtcacttctacgctaacatcttgtcgatggtgtcaaactatgtaacctcgacgctatagaacatgaaatagacctagagtctcgaatcgattgtagttttgtctcaagtctaaacatgtgttgttgccctcttgttttgtattttgttcttttttttgttacgagagaagagatttaaaaaaaacacaagaattgacgtatctgtaacgagcagagtacacacgtgggctagctctccgctgaaaagaatacgatttacatacgtgtaaagattgtgcccactcggcagaaatttggtgatgcgggtccaggtcgttagcctcggatgcagcctgcaggcagcctgtggtgtggtgtggtgtggtccaaaaagggcgggaacagaaacgaggggctggacgcctggacccatggatcaggtggactggtggtccgtgtgggcgcaagcaccagtacagtacagtacttacccccgctcctgcatgcatcgtcctctgtaaacacaaagcacaaggctttacccgaaagcacaagctcacctaattaagctcatgtacgcttctggcgcgcacaatagacacgcccgtacgcaggagcacatggcaccaaccgaatgatttgagcaaccgtctccgcatctggaatccattccactcacccaaacagagctccagctccccctctatccagcgagctggacggacgggacggagcgtagactagcagaacagaagccaggcaggtcgtccggtcggggtcctttccctctttctctccgttttctccgctggggaaaaagaaaatcggaaaatgacgctccacggaagaagcgcgcgagccgatggcaatgcttcccgtcagcgtcgagcggcgagggtccccgagactttttcccccctcctcccctgcgtgccgcacacggccagaacggtccttgctgttgcggctttctatcttggaacagcgccggccggttgaatccgccgtggagtcgagagccgtagtgattcctagtgcaagttgcagagcggagcaaagcaagggaccttgccgcaaaaaccgtggcgggtgtcgtctaactttgtccgtcaagggtcgccgtcggccttgacaaaacggacagctgctgaccgtgacgagttagaagagagagagagagggagatagaagaaaaatcacccacctccggacctccccacacgaaacgaaaagctacgacctacctctcttccagacgtaacgtaagcatgaaacagaaagcactgcctgccggaaaaacaaaaacaaaaacaaaaaaaacccgaaagactaataaataattcaccgctcctctttcgcatttctccggatcttgtatgcatgatgtgtgtgtgtgtgcctgtggataattggacgcactccaccctacagtctcctctctcagcttcgttcctgcgcccccgtatcgtatcctaatgcatgctcacggctgggtcccgtgggccaccaggtttttaatgtgcccttctgtagccacacgcggagaggaaaaggaacccgcaagaaacgggagtggaatagaaggcgtctatttttgcacgatggtataggaagcctgctggcgggctcggcggtgcctcagagtcggcgcgtacaaaatgaagatgcgaagctgttggagttgaccta

SEQ ID NO: 65 - DNA, 1600 bp, Zea mays
  source 1..1600: mol_type = genomic DNA
ttcgtaataataatgcaatactgatggagacgtcgacgactggacacagattcatatataataatgcaatactaatggggcgccctcagtttcagtctttgcgctggaaatggccgatcattaaaaaaatttcttttgtcgaaattcatagacttgcgaaacgatcttgaaatatacttacttcaccatgttcctgcacccaaaaaaaaaagttctcccattcccatcctcttccaggaacaaaagcacagctaccctacccaggtgagggctgagggatgtgtagtagtactgtccatccctgcatggctgcatggcggaatgggcgccggagtcggcggcgcttcgagaatcatgcgtggcaggcagcgagaactccaatgcaaggcagcttgctgccatcgattgccatgactgaaacacgcatgcatgcatgcatgcagagttctagtgtctgcaatggacaatgatgaatcctcctctcccctgcattgcaattgcaaagcagcatgcaatgcaatgctctagatcttccggcgactgggcgccggcgaccacgcccgcccgcccaccaccaaccatacgcatgaattttaagctgcccctcatcaaccagtcatgagtcatcattgccatgcacccccccccccccccccccccccccccggccgcgtcgtccgccgtgcctgccatgccatcgccggaccagacacaatgattcgcccatgatcatcgccggaccggctagtcgatgtggatcgaagcaacgtacgcactgtacgctgtgctgcagtggcaccaccactgtatgtatccactgcaccgcttgttgcgcccacaccaagcacttggtagtttgcatgccccgcagagggtgcaggccggccatgcctgcaggctggctgcagccggctgcatgcatcggccaagcttggctgcagagctagcgatgcatactgggctactggcgtcgcaggcggcggtgatgcgtagtgcggcagtggtcgcggtcgcggtcgcagccggccagcgggaagcgccactggggttttggagagacgtgcatggcgctttctccgggcgctagctagcttagctcgagattgactggcaggctgcatgggcaggcagtgcgcctgcccgcgcctgacgacggctgtgcctcggctagcttgctgccagtgccagtgccaggcaagctgacgcccggtttcctccaccaccggccaccgccatccaggatcaggttcagaagacgagaggaaagtgtgcatgatggagaaatatactgtgagcttcagtttgcccgatgtcacagcagcgcgcgcggtgggcgagggaacaggaggacatgtgctcagctccatgacccggccctccaactgtttacggtcttcgtggccctctaacggttacagtgtgaggatagcgtgcgctccgtcaggccaagtcaaccggatgagtggacgcagcaaaacagtgttgcatgtcacacatgaatagaaattttttttccccttcgcggacccctttttattcaccggtggtgccagtaactctttcctccctacgcttacgtatcaatgtgcagatagctaga

SEQ ID NO: 66 - DNA, 2367 bp, Zea mays; identical to SEQ ID NO: 31
  source 1..2367: mol_type = genomic DNA

SEQ ID NO: 67 - DNA, 2071 bp, Zea mays; identical to SEQ ID NO: 44
  source 1..2071: mol_type = genomic DNA

SEQ ID NO: 68 - DNA, 1577 bp, Zea mays; identical to SEQ ID NO: 46
  source 1..1577: mol_type = genomic DNA

SEQ ID NO: 69 - DNA, 2098 bp, Zea mays
  source 1..2098: mol_type = genomic DNA
acggtcatgggggcacgtgaaagcgtacgcgtgcgcggcgggcgcaccgtgcgcacgttggaccgagcgcggaacggaaggcccgaacccgaagcggccaaacccgccgcggaaaacgtacgagcggggaaggaggcgtgggcgcccatgctgctggggccgccgttagcctcgccggcgcgcgatcgtaccgcgcggcggcggcttttttccatgggcggtgccgccccgccggcgggccctccctagtcccgtcgcaagcggccgctggtttggttggtcgcttcgctcggcagaaagcgagcactgttgacgggttggcgcggcgcggggggctcgcttttcttgccgtcgactcgtggctggctgcaccacctgactggcggcgggcccaccgaggcggggaagtgggaggagacaggccggatccgcgaccggggcggggagggccccccgccccacgtctgtctgctctgggaagtgaaaccaaaccagcagacgaggacgagacaagccagccagccagcccccgtgttgctgccgccggccgctctctgcccgcagcccacagacaggaggggcgtggggggcccgggccggagagagccacctcgcgactcgcgccgtcgccgctcgcaacgactttaactgccacatttatggccctgctataaaacaaactgccgcatttaagacgctaccggcctgagagagattcgggcagaatggggaatgcagatgcatcagtggtgcagcagtgctacagtaccagcgtattgcattgcatgttgcatcgccggaacgcgagcaagattccttcgtcgtgaccgctccacaacgacccttgccttggcttgcactgtcaggtggaagcattgttcactgtcggatacgtacatgagatcatgtgtgcgacagactgtacagcaaacagctagctcacatcacaaaacatgtggccgggcgcacacagactaataagctctcgttaatttagtgtacgccgaccgcgcgcgtcggtctcactttgctgctttgcatcagatcaggtaggtaggtaggttatatatgtcattttgttaagaaccatttgctaattaacaacctgcgccgttttggcgccaaagttttcagacagagttaggtcaaaccaatcctctcttctattttattcatcaacgcgtattaacacaaccaaccacgtatagaacgactcctaaaaagaggctctttcttctgcagttgttttctttctttctcataatgtatgagcttcgaaaagttcagcaattgcttgctgcacttggattacacatgttcattggtcgagtacgtgtcgtttcagctggatttgatgctgcgatgtagtaaccagctatagtgcacgcgtgggccctttttatcacccccaaacccacctttaaggaaatcgtaaaggagatacgtacatctctatctagcttcaacgaaaggtgcgtgccaaaaacaatggcagatgactagattttgtgattggatgatatggaaagcaattattaatcatcttttgatatctcgcggtcggattcatttgattgatagagagccggcgttccacttttcatatttttgcccctactagcagacagtggctctgtaacttccaatatggatttgtgggactgaagggttaattattatctgtattctgtataatcggtggctgtatgtacgatataatcattttgaaacataacggtacattatttctcattcctgtccaagaagcttaagtcatgtatggatctaactaacaacaggctaactaacttcttagtctatgatgatccaaatatacaaactaaaatatgagtccgctagttaataggactagtagttgttagtttcatatatcaatccagttgttactctctggtaacatcaatctaacaagagttcagtccatgctgtttgtctcacgcaaatatatgtgttacgttgaacgccattcgatagtggtatattatcaacttatcttgtggttaatatgattagcaaaagaataacaccatttgtcactatattttacttctatagtataagtagtttttaggaatac

SEQ ID NO: 70 - DNA, 1580 bp, Zea mays; identical to SEQ ID NO: 56
  source 1..1580: mol_type = genomic DNA

SEQ ID NO: 71 - DNA, 1972 bp, Zea mays
  source 1..1972: mol_type = genomic DNA
gctggtgcgcgtctcaaaggcgctcgaggacttccgcgcctccaacgccgagggttcgcaccattcgcctccctttattttggctcctgtttttgaacttcggagcggcaaatttggtgcgttcgaacgagtgtagcagtagtgttgatgcgccgctcagtggactttgcgcctggggctagagtttgagcgtatcccttccaattcttgccccatttggtccttgtgcttttctgttctttttttccaactccattagaacagtgtattttttgtcctgtgatcctgtcgtccttagtcctgagtgcatttgcattacaacctgctttgtggtttgctttgatgtttctgttgtgtactgatcacaatagtgctgctgtttctcatccacattgtgcttttatcaacatgtctgttcacacttgcagttacatataatttgcacatcggctctctgatgctaatattgtcacacaaagtttgaacgttttcttaggtttgacatgtgctaagtggtgcattcagtgtttgtaatgtagctttccagtgatatattttggaattggccaatcttaggttatagaatgcaatcgtctttccacaaacttataactgttttccattgggttgcatcttgcaattttagacatgattgttgtgagatagctgtacgcctcctagataatatagtaaaattgccttgcaaaagtctttgcatgttttcatggctgtatattagtgggagtaccagcacattgagcacaagcacagacccaattaattgtttgatataatttattgatggtagaattctagttacgcgcataagggtcagtgtagcagtagatcttcttattttgatggcatttttttttctcgtatattggctggccctacttcagtttaagcctcacccatctatattatgtagtaatgcattgagattattgctctcttcctttggtgctgtgagctgaataaactgatacttagactattttaccttgcagtgtacacatttgaacctgatatatccaaacaagagcgagctgcaatccatgagatgtgtaggaaaatgggcatgatatccaaaagttctgggtgagtaagataactaggattcattgagcaaaattgtttcatcgaagagttgtaaggaatctatggttatgcttgcgccatcctaatgttatctttacaaaagttacttgttaaatgtctattctgagttctatttaacttcaggaacggggaacgtcgacgcctttctgtttataaaagaaaacagaagcgggggcctgaattggaacaaggccctagctaccttgggttttctgaagaggctaggcatgttttgcaggatttatttatgcattatcctcctggtgatgctgatttaagtggggatgttgaccagagttctagtgataaggctgcaaacatcaaatggagaacagataatgcgttttgcaggcccgcaatgagtaaacttgatataacgaagaaagttgagatgcttgcctcaaaaataaatggatccgaacagttgagaaaggtctcacattctggcttgacaatagtatcttcctctctttgcttagttgctgctacttattagttttatttatgagacaatcttatttatagctaaagcttaaaggtttacttttgaacaggtcatggaagatagaacaaaacttcctatttcatctttcaaggatgtcatcacttcaactttggaaaatcaccaggtatagtttttagctccaaaggtttctcacatttgctagaccacatcttcaactcacatggtgcactggtgctattttttcctctcagatgtgcagggaaactgttttttttttcattaaagaagcatagcataatacaacaatccccaatccccaaggccccaaccaaaaaacagaaaggaaaaactgggtttccactgtgaattttggaacatgcatac

SEQ ID NO: 72 - DNA, 20 bp, Zea mays
  source 1..20: mol_type = other DNA
agcaccggttgctcggaccg

SEQ ID NO: 73 - DNA, 20 bp, Zea mays
  source 1..20: mol_type = other DNA
tacagaaacgcggagagact

SEQ ID NO: 74 - DNA, 20 bp, Zea mays
  source 1..20: mol_type = other DNA
taacgagcagagtacacacg

SEQ ID NO: 75 - DNA, 20 bp, Zea mays
  source 1..20: mol_type = other DNA
tgaaagcgatgcggtttaga

SEQ ID NO: 76 - DNA, 20 bp, Zea mays
  source 1..20: mol_type = other DNA
tacaatgtacagtctagcca

SEQ ID NO: 77 - DNA, 20 bp, Zea mays
  source 1..20: mol_type = other DNA
acgagaccatccaatgatcg

SEQ ID NO: 78 - DNA, 20 bp, Zea mays
  source 1..20: mol_type = other DNA
tggagagtaataggatggca

SEQ ID NO: 79 - DNA, 20 bp, Zea mays
  source 1..20: mol_type = other DNA
tgaaaccaaaccagcagacg

SEQ ID NO: 80 - DNA, 20 bp, Zea mays
  source 1..20: mol_type = other DNA
taggtttgacatgtgctaag

SEQ ID NO: 81 - DNA, 20 bp, Zea mays
  source 1..20: mol_type = genomic DNA
cttcgtagacatatagatgc

SEQ ID NO: 82 - DNA, 988 bp, Zea mays
  source 1..988: mol_type = other DNA
ccgtggagaaaactggttgggctgtgccgagaaacaatgagcgggctgggctgcgtcttgacggcgggccaccaaactgtcccgccgtgcgcgcagggcaacaaccacaacgtcatgtgcggcttgcttaactgggcccgttagacgcgggcttcgtgtggccttggaacacggccgttcgacctggcttcacgtgacgtgaaccagagcggagcggggccatcgatttcggccgcgcgaaacgcgtgcgcgaggcctgcgaaaggccgctggatgaagctccctttgattgaagcccgtgtgggccgcaccgcatggtccggccggcgatcgtgaccgttggagtacgatttattcgatgcgtatgtactcagctcgatccatatacgatatgatagtacgtagacatcttagacgtaagttgtttaaggaactctctctctctctctctctctctctcgggtttctgtgttcatctcaaagttttttcagttcaaaaaccaattcgaaaacaaatcggcttaaaattcaggtaatcaggtcaagcgactttactctggtctgaataacttgagacatccgggttgccatggccgactctagacagcggccataaacacggtggtttctttttcttattgggatagtaggtcactccaaataaaggctattgccatatgctaaggagacggaatttgtgacgccatcgccaccgggttaacgttaatattctactactagagaatctagcttacgtttcggttccggccggccagtagaaaactctctctgaaccgaccggtcagaatcccctgctcggtgctcggttgcttggaccgcacgcacgcacccctatatcgtcagtgcctgtaacagttcttattcggtgattattattataatattattccacgtttgcacacaccgcacatccgcccgtttgtaaaactgtgagttgatcgtcgagacgaaaggtggagctggagta

SEQ ID NO: 83 - DNA, 1001 bp, Zea mays
  source 1..1001: mol_type = other DNA
ctctccgcgtttctgtaactagaattgtcatagtcagggttgccaaacatcagcatcccgaggcagtttctttaattctgcttttttttatatatgtaagtttgcttaccgaatgagctagttctaaacaaactcaaaaacaaaacagggcaactgggggtcccttgacattgcacagatggacctgaccactttgagattcccccggcttctatctccttttccctccccttggatcaaatgaacaaaggagcgcattctctctctctctctctctctaaaagattaaaaaaaagcctgcatgtagtgttctttgacaaggacaaggaagcccttttacatcaatacatcattcgtatgttgttgttttctgtgttctttgcgttcctttttttcccctccctccgccttttttctacttgattgttgccaagatctggagcacctgctctgatctgattgtgtgcgctggtttactgaacctttcggagggctatacgcttcgtacggggacataccaatttcaaagaattcagtcatcaggtaggtggttcaatcataccgatggtttcctcactgcatcactcaccttctcatttttacgcatcataattttttgttcccttctccttaattcccatgcggtgaaggagagatgtgaactaacagtttggcgctgcactgttcgaccggctaaacacggggccaatgctctctgtacgtgcagatggataggatagtctttgattcttgtttcaagatgacgtggatagtctataatagctaaatgtttgcctcgactactaacttgccgatatgggcgagggtaactttaaattaaatttttaaagcatttgacttgttaaaaaaaataaaagcctatattctttgttgatggagggagcaagtggctgaaaagccgttgccatttctgggcgctccctaaactacgcggcaagcaggctattgggagcccttgtcgctgtcgacgcgatgtgcgg

SEQ ID NO: 84 - DNA, 1000 bp, Zea mays
  source 1..1000: mol_type = other DNA
acgtgggctagctctccgctgaaaagaatacgatttacatacgtgtaaaggttgtgcccactcggcagaaatttggtgatgcggggccaggtcgttagcctcggatgcagcctgcaggcagcctgtggtgtggtgtggtccaaaaagggcgggaacagaaacgaggggctggacgcctggacccatggatcaggtggtggtggtccgtgtgggcgcaagcaccagtacagtacagtacagtacttccccccccgctcctgcatgcatcgtcctctgtaaacacaaggctttacccgaaagcacaagctcacctaattaagctcatgtacgcttctggcgcgcacaatagacacgcccgtacgcaggagcacatggcaccaaccgaatgatttgagcaaccgtctccgcatctggaatccattccactcacccaaacagagctccagctccccctctatccagcgagctggacgggacgggacggagcgtagactagcagaacagaagccaggcaggtcgtccggtcggggtcctttccctctttctctccgttttctccgctggggaaaaagaaaatcggaaaatgacgctccacggaagaagcgcgcgagccgatggcaatggttcccgtcagcgtcgagcggcgatggtccccgagactttttccccccctcctcccctgcgtgccgcacacggccggaacggtccttgctgttgcggctttctatcttggaacagcgccggccggttgaatccgccgtgttcctagtgcaagttgcagagcggagcaaagcaagggaccttgccgcaaaaaccgtggcggggtgtcgtctaactttgtccgtcaagggtcgccgtcggccttgacaaaacggacagctgctgaccgtgacgagttagaagagagagagagagggagatagaagaaaaatcacccacctccggacctccccacacgaaacgaaaagctacgacctacctctcttccagacgtaacgta

SEQ ID NO: 85 - DNA, 990 bp, Zea mays
  source 1..990: mol_type = other DNA
aaaccgcatcgctttcaggctttatttaatctctgcgtcaaggaaaggagcaatcttctgcgagaaaagaagaaggtctcgcgtccatgtatgcagttgaactgtttactcatgagaatgagaggggggagagagacatcagacatcccagtgaaggtttcagagacgcgactgttgatccgatggccccggcccctcatcagcaataatcagctggaccgaaaaagatacacgtactgggattttccatgtaccccccaaaaacaatattagctcatgcatgaactgggcgattatatcgtcatttaatttgtgggttacatgttgtttatgcgtagagagaagatagagttggtagcaagtcccggtttgtgcatagcaagatgtgatttttgtccattaattcgtcagtaggtgatgattgactctgacgatgagaccgacaggcggacgcatatattatgcatcgatgtgcattctgtgtgtgatttgtgactcaatattattcctctggctagtcttgttcccggccatttctccctgaattcccctctgatctttttttaatattttatgtaaagaaagaaaaaaacactgaaggccgacgatatgcgcgtgaggacgacaatcgcacacgtgtttgcatttgataacattttatcttgtctagcagacattagtttgattaacttgtttcatgccaatcattaatttggagtatatatgattgatatcttattatcctgtccgtttgtatattatttaagtagcatggattggagtactacaccgttaacaatacattccacaaatacctttccctccatccaaattaaggcttaattttagataatgacgcatcttcttcctatagcccaccctatgcacaggtaagcctcacaaggtacgtagtttttacactaattattagctttgcaaattgcaatatagctacaagttttgcatcggctgatttttttgattaattgg

SEQ ID NO: 86 - DNA, 993 bp, Zea mays
  source 1..993: mol_type = other DNA
ctagactgtacattgtacttgaatactctgggtgaccaacccccagaacacgtccttctatctacagatggcaattaaaaaaagataaattgcatacttcaacaagtgaattacaagcacatttacatttccattgttttgctaccatgctttgatgctttcattgaatgggtacaggtgttaagcagcagagtgacagaagatgacaacatatattcgtatagttctgaaaggttatattttaacctatttgtatgcgatcaaccaaaaatggacaattataagtatggtcaagtaagatgatagtatagcataactcaaatattgttattgtggaatatcacattggtatcaattatgacagaggcacaaaatttcaagaataaatgttaaacataaataagcaagagcacaaaaagttgaggtgtggtaccttcaaagcacgctgcaaaaggggggaaacaacaaacagaaaagggtataagaagcaagttcaaatcttcgaatgaaaaaaattaagttcaagacttggaatgaaaaaattaagtgcagaaagtatagcagcagttgacacaaagggtataatgctacaatggaaacagttcaattagcctttcacaacttcatgagaaaaatacatcaaggatctcccctactgtgacttcgttactaaatttcattctgtgtatcatgataatgatgcatatagtgaggactagcatgcctcaaacaaaaagctgcctacaaaaatgtccattcgctcacttgcagcttgaacagagtattagcatgcaaaattttgcacagatttacaaatttcttagattatgattcccatcaaaggtaacagatattgtcattcctacaaactattgcatgcggtctgttttcaaactgtttaatgatattcaccaaaaaagacaaaaacaaacttttcaaactgtgttgcatagcaaaattacagaagaaactataaaatctaaacaaagaaga

SEQ ID NO: 87 - DNA, 998 bp, Zea mays
  source 1..998: mol_type = other DNA
tcattggatggtctcgtacgtaagtttactacccgacgttgcaccgcgaaattcaaatgccagtgccgaactaagtttggctgtcatacatcgtcagaaggaaaacagaacccccaaaaaaaaacataatgaggtattcccatcagaggtaagcagcttaggcggctgagttgaatttgactgaataatatccatgcttgtgtgtgcgtgcctaacgcattgtcgtcgtgttctacgtacgctgctgcatgctactactttgctactaaaagtcatgcatcgtctcaggtagagctaggctctaataatgtactagtttatttgaaacgtacgtccggcatacggaacaagtagtaataatgagcatcagatcgcgtgcaatgcagaggatagcagaatatacttgtatgtagctatgtataggccacgacttgcgtaaagctctaacggctgttcacgaccccaacgtcgtacgagaggccgggaaacaatcgtccagcaggccggcggccggctagctcggttgccgtaagctacgattcttagcacattaatattacattacatgcatgcatatcccgcaccgctgtagcatctatacacagagtactccacatctacatctcctggaggtcgatcgacctggctagtactagtagtacacaggccgcgggcgcgcgcgtttggtgcacgtcgtacacaacccccttgaggaataatcgcatgcctcgacgacgacgacgtcggagtcagcggcgtggcaacaatgttggttagttgggcaaattaaattgaactcccgaagagagacggttgaactgatgactgagatgagtcgtcgtaaacgtgtgtgccaaggttgggcagggttgggttgggttgggccgggcgtacgtatacgtctctgatgcatcgtccccctaaggacggccgacgacacacatctgtgacagtaactgacaaggactgcatcactacgacgatgcacgcgtcattcg

SEQ ID NO: 88 - DNA, 999 bp, Zea mays
  source 1..999: mol_type = other DNA
gcaaggagtattttttgaaatttctaggcgttttcgaaataatcataaatgttggtggttgtgatgccttttatgtaaccgcgtagaagaagagagagagatagctatgcttatgataaagaagatgtgtgttattgtttgtatatgataatttttatttaccatgtgggtattgatagtgatgcctacatgatgaatcattttgaggagcaatgcttgttttcattattggccccaatgttttccaagattggacattatggaaggccttgacctcccaccggtttattaggtaggtctcctttatcttgcactaagacttctcatgttcatatccttaggcattggacatatggaccatacaaaggtgaaaggaccatcaagatcctatttaggtccattaaatatatatgtagttagaagtagaagtggcaagtcaagaggactggtatgtcatacgtcacatgggtgcatgtcacatcggagtgagctagcgctatcaagaagaaaataaaggtaaatgtagcaatgggtatattagtctactatttgtacatgtgatcctgaaagagacattaatatgttggcctagattggacttcggtcttataggacatgataagtaataactgaaactactttcccgttccgtttatcttatttttcttcttcgcttcttccatctctatccctatccctatccctctctctctcttttaaattcttccccaaatatacatatatatccctattgcatccctggatcgaaagggacatgacaattcgtatgagatctaggctcttcatgcaggtaattcctttattaccctcttggtcttgagtgacaatcatcattaactagtgttttcattagacttgcacttccattccaggtctgtaatctagtcattttggactagagcaattcaccatcacttgtaattggtaagcaacttattctttttaatttgcactatcatggaggatgtggaagcgtga

SEQ ID NO: 89 - DNA, 997 bp, Zea mays
  source 1..997: mol_type = other DNA
acgaggacgagacaagccagcagccagctcccgtgttgctgccgccggccgctctctgcccgcagcccgcagcccacagacaggaggggcgtggggggcccgggccggagagagccacctcgcgactcgcgccgtcgccgctcgcaacgactttaactgccacatttatggccctgctataaaacaaactgccgcatttaataggctaccggccaggcctgagagagattcgggcagaatggggaatgcagatgcatcagtggtgcagcagtgctacagtaccagcgtattgcattgcatcgccggaacgcgagcaagattccttcgtcgtgaccgctccaaaacgacccttgccttggcttgcactgtcaggtggaagcattgttcactgtcggatacatacatgagatcatgtgtgcgacagactgtacagcaaacagctagctcacatcacaaaacatgtggccggggcgcacacagactaataagctctcgttaatttagtgtacgccgaccgcgcgcgtcggtctcactttgctgctttgcatcagatcaggtaggtaggtaggttatatatgtcattttgttaagaaccatttgctaattaacaacctgcgccgttttggcgccaaagttttcagacagagttaggtcaaaccaatcctctcttctattttattcatcaacgcgtattaacacaaccaaccacgtatagaacgactcctaaaaagaggctctttcttctgcagttgttttttttttctcataatgtatgagcttcgaaaagttcagcaattgcttgctgcacttggattacacatgttcattggtcgagtacgtgtcgtttcagctggatttgatgctgcgatgtagtaaccagctatagtgcacgcgtgggccctttttatcacccccaaacccacctttaaggaaatcgtaaaggagatacgtacatctctatctagcttcaacgaaaggtgcgtgccaaaaacaatgg

SEQ ID NO: 90 - DNA, 999 bp, Zea mays
  source 1..999: mol_type = other DNA
aagtggtgcattcagtgtttgtaatgtagctttccagtgatatattttggaatctcccaatcttaggttatagaatgcaatcgtctttccacaaacttataactgtttaccattgggttgcatcttgcaattttagacatgattgttgttagatacctgtacgcctcctagatatatagtaaaattgccttgcaaaagtctttgcatgttttcatggctgtatattagtgggagtaccagcacattgagcacaagcacagacccaattaattgtttgatataatttattgatggtagaattctagttacgcgcataagggtcagtgtagcagtagatcttattttgatggcattctttttctcgtatattggctggccctacttcagttgaagcctcacccatctatattatgtagtaatgcattgagattattgctctcttcctttggtgctgtgagctgaataaactgatacttagactattttaccttgcagtgtacacatttgaacctgatatatccaaacaagagcgagctgcaatccatgagatgtgtaggaaaatgggcatgatatccaaaagttctgggtgagtaagataactaggattcattgagcaaaattgtttcatcgaagagttgtaaggaatctatggttttgcttgcgccatcctaatgttatctttacaaaagttacttgttaaatgtctattctgagttctatttgacttcaggaacggggaacgtcgacgcctttctgtttataaaagaaaacagaagcgggggcctgaattggaacaaggccctagctaccttgggttttctgaagaggctaggcatgttttgcaggatttatttatgcattatcctcctggtgatgctgatttaagtggggatgttgaccagaattctagtgataaggctgcaaacatcaaatggagaacagatagtgcgttttgcaggcccacaatgagtaaacttgatataacgaagaaagttgaga

SEQ ID NO: 91 - DNA, 986 bp, Zea mays
  source 1..986: mol_type = other DNA
tgctggtattgattgagatgtggctagagtgatcggccggcatgcatgcgatgcgatgcgttgcgacggaggggagatccttctcctgctatctcgtctcgtctctatactagctccgcgccaagaacgtcgtcacagaacgtacttactaccagcagggggccgggcccgggaggggctctggccggtcgacagatcgttggcaaccaccgccggccactcattgagcgagatgcatgggctagtaggctaatcctatattttttttaacacgcatgcatatgctgatcaatgcgaacagacggactgaactcgacccatctggatgtagcagtagtggtggctgctgtttggctggcatggcatggcatgcgtccggcaggcacgcatgacagcatccatccagtaccttattgtctacatcgaatattttgaaaattttaaattcgctggttttcgtcgactggctatatatagcgcatggaaatgaatttaatttagctgcagatggaaggttgttagaggacgaaggagctcaaatcgagcttttgcatggaataatttatacgttcccagcatatatatgcatacatagacatagagctgaaccggcgcatgtctacctactacatacaaacgaggcagatgaggttgctagtagctcgacgatcatgcccttgttcttctttattccccatttaatagcaaatagagagagagagagattaaaaaaagggaaattgttcagtcaggagtcaaattccttctctatctagaggcaatacacgcagtatatatacacatatctctgtcaccggattattgaaacttttgtttaagtagtatagtagtatatagcaagcaagattttaaaattttgggtgccctcaactatacatacaaaattcaccattcacatggcctgcttgccactccttcggtccttaagagctgctgtgccctgccctgcgcagactgcatgagcg

SEQ ID NO: 92 - DNA, 22 bp, Zea mays
  source 1..22: mol_type = other DNA
tgacaagaccacacacacgcat

SEQ ID NO: 93 - DNA, 23 bp, Zea mays
  source 1..23: mol_type = other DNA
gtgacggactgacggtgagtatc

SEQ ID NO: 94 - DNA, 23 bp, Zea mays
  source 1..23: mol_type = other DNA
atgatgtgtgtgtgtgtgcctgt

SEQ ID NO: 95 - DNA, 21 bp, Zea mays
  source 1..21: mol_type = other DNA
tgcccacttttgctttcacat

SEQ ID NO: 96 - DNA, 23 bp, Zea mays
  source 1..23: mol_type = other DNA
tgaactgttgaaacaccaaatgc

SEQ ID NO: 97 - DNA, 20 bp, Zea mays
  source 1..20: mol_type = other DNA
tcagcaccggccgaacttat

SEQ ID NO: 98 - DNA, 22 bp, Zea mays
  source 1..22: mol_type = other DNA
gctttgcttgcacttggttgat

SEQ ID NO: 99 - DNA, 25 bp, Zea mays
  source 1..25: mol_type = other DNA
gcttcttggacaggaatgagaaata

SEQ ID NO: 100 - DNA, 24 bp, Zea mays
  source 1..24: mol_type = other DNA
agcagcaactaagcaaagagagga

SEQ ID NO: 101 - DNA, 19 bp, Zea mays
  source 1..19: mol_type = other DNA
aagcagcaggattgggagc

SEQ ID NO: 102 - DNA, 21 bp, Zea mays
  source 1..21: mol_type = other DNA
tgtgctgcaaggcgattaagt

SEQ ID NO: 103 - DNA, 21 bp, Zea mays
  source 1..21: mol_type = other DNA
tgtgctgcaaggcgattaagt

SEQ ID NO: 104 - DNA, 21 bp, Zea mays
  source 1..21: mol_type = other DNA
tgtgctgcaaggcgattaagt

SEQ ID NO: 105 - DNA, 20 bp, Zea mays
  source 1..20: mol_type = other DNA
ccgggtgaatcagcgtttat

(19) United States
(12) Patent Application Publication
(10) Pub. No.: US 2025/0137004 A1
(43) Pub. Date: May 1, 2025

(54) TARGETED INSERTION SITES IN THE MAIZE GENOME

(71) Applicant: SYNGENTA CROP PROTECTION AG, Basel (CH)

(72) Inventors: Yinping Lucy Qin, Durham, NC (US); Mark Rose, Durham, NC (US); Zhongying Chen, Durham, NC (US); Heng Zhong, Durham, NC (US); Weining Gu, Durham, NC (US); Wenling Wang, Durham, NC (US); Qiudeng Que, Durham, NC (US); Ailing Zhou, Durham, NC (US); Mary-Dell Chilton, Durham, NC (US)

(73) Assignee: SYNGENTA CROP PROTECTION AG, Basel (CH)

(21) Appl. No.: 18/985,681

(22) Filed: Dec. 18, 2024

Related U.S. Application Data
(62) Division of application No. 17/822,537, filed on Aug. 26, 2022, now Pat. No. 12,209,247.
Application No. 17/822,537 is a division of application No. 16/757,851, filed on Apr. 21, 2020, now Pat. No. 11,459,577.
(60) Provisional application No. 62/599,831, filed on Dec. 18, 2017.

(51) Int. Cl. C12N 15/82; A01H 6/46

(52) U.S. Cl. CPC C12N 15/8213; A01H 6/4684

(57) ABSTRACT

The present invention relates to methods and compositions for targeted insertion of polynucleotide molecules into ideal target sites in the genome of a maize plant. The present invention relates to maize recombinant molecules comprising heterologous sequences and also to methods of integrating a DNA of interest into a target maize genomic locus in a maize genome. The present invention also relates to regenerated maize plants or plant parts comprising the recombinant molecules and/or a DNA of interest.

### RELATED APPLICATIONS

This application claims the benefit of provisional application 62/599,831 filed Dec. 18, 2017 and incorporated by reference in its entirety herein.

### STATEMENT REGARDING ELECTRONIC SUBMISSION OF A SEQUENCE LISTING

A sequence listing in ASCII text format, submitted under 37 C.F.R. § 1.821, entitled “81487_ST25.txt”, 206 kilobytes in size, generated on Dec. 14, 2017 and filed via EFS-Web is provided in lieu of a paper copy. This sequence listing is hereby incorporated by reference into the specification for its disclosures.

### BACKGROUND OF THE INVENTION

Recent advances in the field of targeted genomic modifications have made it so that routine targeted modifications for agrobiotechnological approaches may soon be possible. Significant advances include the development of methods and compositions to target and cleave genomic DNA by site specific nucleases (e.g., Zinc Finger Nucleases (ZFNs), Meganucleases, Transcription Activator-Like Effector Nucleases (TALENS) and Clustered Regularly Interspaced Short Palindromic Repeats/CRISPR-associated nucleases (CRISPR/Cas) with an engineered crRNA/tracr RNA), to induce targeted mutagenesis, to induce targeted deletions of DNA sequences, and to facilitate targeted recombination of an exogenous donor DNA polynucleotide, such as a transgene, within a predetermined genomic locus. However, this predetermined genomic locus is not obvious. Many sites in the genome are non-ideal for targeted genomic modifications, particularly for targeted insertion of a DNA of interest, due a number of factors, including highly repetitive nucleotide sequences, methylation, chromatin structure, epigenetic modifications such as acetylation, and other characteristics that result in a high level of recombination or a poor level of expression of introduced coding sequences. Therefore, there is a need in the art to identify ideal target sites within a genome for targeted modifications such as transgene insertion. The present invention addresses these shortcomings in the art by providing ideal target sites for a maize genome.

### SUMMARY OF THE INVENTION

The present invention provides a method of integrating a DNA of interest into a target maize genomic locus, wherein the target maize genomic locus comprises a nucleic acid sequence of at least 100, at least 500, at least 750, at least 1,000, at least 1,250, or at least 1,500 contiguous nucleotides having at least 70%, at least 75%, at least 80%, at least 85%, at least 90%, at least 93%, at least 95%, at least 96%, at least 97%, at least 98%, at least 99%, or 100% identity to at least 100, at least 500, at least 750, at least 1,000, at least 1,250, or at least 1,500 contiguous nucleotides of SEQ ID NO: 1 through SEQ ID NO: 71, or a complement thereof, comprising introducing into a maize cell: (a) a first nucleic acid molecule comprising at least 50, at least 60, at least 70, at least 80, at least 90, at least 100, at least 110, at least 120, at least 130, at least 140, or at least 150 contiguous nucleotides, wherein said contiguous nucleotides have at least 80% identity to at least 100, at least 110, at least 120, at least 130, at least 140, or at least a 150 contiguous nucleotides of SEQ ID NO: 1 through SEQ ID NO: 71, or a complement thereof, and further comprising a DNA of interest; and (b) a second nucleic acid molecule comprising a nucleotide sequence encoding a nuclease for site-directed cleavage at a genomic nuclease cleavage site of, adjacent to, or proximal to the genomic nucleotide sequence of SEQ ID NO: 1 through SEQ ID NO: 71, or a complement thereof, that corresponds to the contiguous nucleotides of (a), under conditions wherein expression of the second nucleic acid molecule can occur to produce the nuclease and the nuclease can cleave the nucleotide sequence at the genomic nuclease cleavage site, whereby the DNA of interest is integrated at the genomic nuclease target cleavage site in the maize genome.

In some embodiments, the first nucleic acid molecule and the second nucleic acid molecule can be introduced into the maize cell by biolistic nucleic acid delivery, via an Agrobacterium, by co-transformation, and/or with a T-DNA vector in any combination and/or order. In some embodiments, the first nucleic acid molecule and the second nucleic acid molecule can be present on a single nucleic acid construct or on separate nucleic acid constructs.

In some embodiments, the first nucleic acid molecule and/or the second nucleic acid molecule can be transiently expressed in the maize cell or can be stably integrated into the maize genome of the maize cell.

In some embodiments, the DNA of interest comprises at least one expression cassette. In some embodiments, the DNA of interest comprises a transgene.

In another embodiment, the present invention provides a method of making a maize plant cell comprising a DNA of interest, said method comprising: (a) selecting a target maize genomic locus, wherein the target maize genomic locus comprises a nucleic acid sequence of at least 100, at least 500, at least 750, at least 1,000, at least 1,250, or at least 1,500 contiguous nucleotides having at least 70%, at least 75%, at least 80%, at least 85%, at least 90%, at least 93%, at least 95%, at least 96%, at least 97%, at least 98%, at least 99%, or 100% identity to at least 100, at least 500, at least 750, at least 1,000, at least 1,250, or at least 1,500 contiguous nucleotides contiguous nucleotides of SEQ ID NO: 1-71 or a complement thereof; (b) selecting a site specific nuclease that specifically binds to and cleaves a genomic nuclease cleavage site within said target maize genomic locus; (c) introducing said site specific nuclease and a DNA of interest into the maize plant cell; (d) allowing the DNA of interest to insert into the target maize genomic locus; and (e) selecting maize plant cells that comprise the DNA of interest inserted into the target maize genomic locus.

The site specific nuclease of the method described above may be introduced into the maize plant cell either as a polypeptide or as nucleic acid molecule, which is transcribed and/or translated in the plant cell to produce the site specific nuclease. The site specific nuclease may be transiently expressed in the plant cell. The site specific nuclease may not be expressed in the maize cell, and may only be present in the maize cell as an active nuclease. The site specific nuclease and the DNA of interest may be introduced into the cell simultaneously or not simultaneously.

In some embodiments of the methods of the invention, the site specific nuclease is a CRISPR-associated nuclease, a zinc finger nuclease, a TALEN, or a meganuclease, singly or in combination. In some embodiments of the methods of the invention, the site specific nuclease is a CRISPR-associated nuclease, such as Cas9 or Cpf1, and the method includes at least one additional nucleic acid molecule encoding a guide RNA, which is also introduced into the maize cell. The guide RNA may be a single guide RNA or a dual guide RNA. The additional nucleic acid molecule(s) may be DNA molecule(s) that can be expressed in the maize cell to produce the guide RNA, or it may be RNA molecule(s), the guide RNA itself, which is introduced into the maize cell.

The present invention further provides a method of producing a maize plant, plant part, or progeny thereof comprising a DNA of interest, such as a transgene, integrated into the genomic nuclease cleavage site in the maize genome, comprising regenerating a maize plant from the maize cell produced by the method described herein. Accordingly, the present invention provides a maize plant, plant part, or progeny thereof comprising the transgene integrated into the genomic nuclease cleavage site in the maize genome, produced by the method of this invention.

The present invention also provides a maize recombinant polynucleotide, wherein the recombinant polynucleotide comprises a nucleic acid sequence of at least 50 nucleotides, at least 60, 70, 80, 90, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or at least 20,000 nucleotides, and has at least 70%, at least 75%, at least 80%, at least 85%, at least 90%, at least 93%, at least 95%, at least 96%, at least 97%, at least 98%, at least 99%, or 100% identity to at least 50 contiguous nucleotides, 60, 70, 80, 90, 100, 150, 200, 250, 300, 250, 400, 450, 500, 600, 700, 800, 900, 1000, 1500, 2000, 2500, 2750, 2900, or at least 3000 contiguous nucleotides of SEQ ID NO: 1-71 or a complement thereof, and wherein the recombinant polynucleotide further comprises a DNA of interest, wherein the DNA of interest is inserted into the nucleic acid sequence to produce said recombinant polynucleotide.

In some embodiments, the recombinant polynucleotide comprises a DNA of interest which is a transgene. In some embodiments, the DNA of interest comprises at least one gene of interest. In some embodiments, the DNA of interest comprises at least one expression cassette. In some embodiments, the DNA of interest comprises two or more expression cassettes. In some embodiments, the DNA of interest does not encode a polypeptide. In some embodiments, the DNA of interest comprises regulatory sequences.

The present invention also provides a maize plant, plant part, or plant cell comprising the recombinant polynucleotide described above.

### BRIEF DESCRIPTION OF THE SEQUENCES IN THE SEQUENCE LISTING

The nucleic acid sequences listed in the accompanying sequence listing are shown using standard letter abbreviations for nucleotide bases, as defined in 37 C.F.R. § 1.822. The nucleic acid and amino acid sequences listed define molecules (i.e., polynucleotides and polypeptides, respectively) having the nucleotide and amino acid monomers arranged in the manner described. The nucleic acid and amino acid sequences listed also each define a genus of polynucleotides or polypeptides that comprise the nucleotide and amino acid monomers arranged in the manner described.

Only one strand of each nucleic acid sequence is shown, but the complementary strand is understood as included by any reference to the displayed strand. As the complement and reverse complement of a primary nucleic acid sequence are necessarily disclosed by the primary sequence, the complementary sequence and reverse complementary sequence reference to the nucleic acid sequence, unless it is explicitly stated to be otherwise (or it is clear to be otherwise from the context in which the sequence appears). Furthermore, as it is understood in the art that the nucleotide sequence of an RNA strand is determined by the sequence of the DNA from which it was transcribed (but for the substitution of uracil (U) nucleobases for thymine (T)), an RNA sequence is included by any reference to the DNA sequence encoding it. In the accompanying sequence listing:

SEQ ID NOs: 1 to 61 are nucleotide sequences of identified safe harbor sites, ideal for targeted integration, within the genome of the maize elite inbred line AX5707.

SEQ ID NOs: 62 to 71 are nucleotide sequences of identified safe harbor sites, ideal for targeted integration, within the genome of the maize variety B73.

SEQ ID NOs: 72 to 81 are nucleotide sequences of target maize genomic loci for targeted insertion of a DNA of interest using the CRISPR-Cas9 system

SEQ ID NOs: 82 to 91 are nucleotide sequences of maize genomic fragments from the elite inbred line AX5707 which can be used as homologous arms for recombination into a safe harbor site. SEQ ID NOs: 92 to 105 are primers useful to the invention.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The following definitions and methods are provided to better define the present invention and to guide those of ordinary skill in the art in the practice of the present invention. Unless otherwise noted, terms used herein are to be understood according to conventional usage by those of ordinary skill in the relevant art. Definitions of common terms in molecular biology may also be found in Rieger et al., Glossary of Genetics: Classical and Molecular, 5^{th} edition, Springer-Verlag: New York, 1994.

The terms “nucleotide sequence” “nucleic acid,” “nucleic acid sequence,” “nucleic acid molecule,” “oligonucleotide” and “polynucleotide” are used interchangeably herein to refer to a heteropolymer of nucleotides and encompass both RNA and DNA, including cDNA, genomic DNA, mRNA, synthetic (e.g., chemically synthesized) DNA or RNA and chimeras of RNA and DNA. The term nucleic acid molecule refers to a chain of nucleotides without regard to length of the chain. The nucleotides contain a sugar, phosphate and a base which is either a purine or pyrimidine. A nucleic acid molecule can be double-stranded or single-stranded. Where single-stranded, the nucleic acid molecule can be a sense strand or an antisense strand. A nucleic acid molecule can be synthesized using oligonucleotide analogs or derivatives (e.g., inosine or phosphorothioate nucleotides). Such oligonucleotides can be used, for example, to prepare nucleic acid molecules that have altered base-pairing abilities or increased resistance to nucleases. Nucleic acid sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. Unless otherwise indicated, a particular nucleic acid sequence of this invention also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid molecule is used interchangeably with gene, cDNA, and mRNA encoded by a gene.

The term “derived from” in the context of a molecule refers to a molecule isolated or made using a parent molecule or information from that parent molecule. For example, a Cas9 single mutant nickase and a Cas9 double mutant null-nuclease are derived from a wild-type Cas9 protein.

A “coding sequence” is a nucleic acid sequence that is transcribed into RNA such as mRNA, rRNA, tRNA, snRNA, sense RNA or antisense RNA. In some embodiments, the RNA is then translated in an organism to produce a protein.

By the term “express” or “expression” of a polynucleotide coding sequence, it is meant that the sequence is transcribed, and optionally translated.

“Expression cassette” as used herein means a nucleic acid molecule capable of directing expression of a particular nucleotide sequence in an appropriate host cell, comprising a promoter operably linked to the nucleotide sequence of interest, typically a coding region, which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette may also comprise sequences not necessary in the direct expression of a nucleotide sequence of interest but which are present due to convenient restriction sites for removal of the cassette from an expression vector. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation process known in the art. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue, or organ, or stage of development. An expression cassette, or fragment thereof, can also be referred to as “inserted sequence” or “insertion sequence” when transformed into a plant.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). In some embodiments, a gene refers to only the coding region. A gene may be “isolated” by which is meant a nucleic acid molecule that is substantially or essentially free from components normally found in association with the nucleic acid molecule in its natural state. Such components include other cellular material, culture medium from recombinant production, and/or various chemicals used in chemically synthesizing the nucleic acid molecule.

The term “native gene” refers to a gene as found in nature. The term “chimeric gene” refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Promoter” refers to a nucleotide sequence, usually upstream (5′) to its coding sequence, which controls the expression of the coding sequence by providing the recognition for RNA polymerase and other factors required for proper transcription. “Promoter regulatory sequences” consist of proximal and more distal upstream elements. Promoter regulatory sequences influence the transcription, RNA processing or stability, or translation of the associated coding sequence. Regulatory sequences include enhancers, promoters, untranslated leader sequences, introns, and polyadenylation signal sequences. They include natural and synthetic sequences as well as sequences that may be a combination of synthetic and natural sequences. An “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. The meaning of the term “promoter” includes “promoter regulatory sequences.”

“Intron” refers to an intervening section of DNA which occurs almost exclusively within a eukaryotic gene, but which is not translated to amino acid sequences in the gene product. The introns are removed from the pre-mature mRNA through a process called splicing, which leaves the exons untouched, to form an mRNA. For purposes of the present invention, the definition of the term “intron” includes modifications to the nucleotide sequence of an intron derived from a target gene, provided the modified intron does not significantly reduce the activity of its associated 5′ regulatory sequence.

“Exon” refers to a section of DNA which carries the coding sequence for a protein or part of it. Exons are separated by intervening, non-coding sequences (introns). For purposes of the present invention, the definition of the term “exon” includes modifications to the nucleotide sequence of an exon derived from a target gene, provided the modified exon does not significantly reduce the activity of its associated 5′ regulatory sequence.

A “transgene” refers to a nucleic acid molecule that has been introduced into the genome by transformation and is stably maintained. A transgene may comprise at least one expression cassette, typically comprises at least two expression cassettes, and may comprise ten or more expression cassettes. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

As used herein the term transgenic “event” refers to a recombinant plant produced by transformation and regeneration of a single plant cell with heterologous DNA, for example, an expression cassette that includes one or more genes of interest (e.g., transgenes). The term “event” refers to the original transformant and/or progeny of the transformant that include the heterologous DNA. The term “event” also refers to progeny produced by a sexual outcross between the transformant and another line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. For example, “event MIR604,” “MIR604” or “MIR604 event” as used herein, means the original MIR604 transformant and/or progeny of the MIR604 transformant (U.S. Pat. Nos. 7,361,813, 7,897,748, 8,354,519, and 8,884,102, incorporated by references herein).

As used herein, “heterologous” refers to a nucleic acid molecule or nucleotide sequence not naturally associated with a host cell into which it is introduced, that either originates from another species or is from the same species or organism but is modified from either its original form or the form primarily expressed in the cell, including non-naturally occurring multiple copies of a naturally occurring nucleic acid sequence. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., present in a different copy number, and/or under the control of different regulatory sequences than that found in the native state of the nucleic acid molecule. A nucleic acid sequence can also be heterologous to other nucleic acid sequences with which it may be associated, for example in a nucleic acid construct, such as e.g., an expression vector. As one nonlimiting example, a promoter may be present in a nucleic acid construct in combination with one or more regulatory element and/or coding sequences that do not naturally occur in association with that particular promoter, i.e.,, they are heterologous to the promoter.

An “isolated” nucleic acid molecule or nucleotide sequence or an “isolated” polypeptide is a nucleic acid molecule, nucleotide sequence or polypeptide that, by the hand of man, exists apart from its native environment and/or has a function that is different, modified, modulated and/or altered as compared to its function in its native environment and is therefore not a product of nature. An isolated nucleic acid molecule or isolated polypeptide may exist in a purified form or may exist in a non-native environment such as, for example, a recombinant host cell. Thus, for example, with respect to polynucleotides, the term isolated means that it is separated from the chromosome and/or cell in which it naturally occurs. A polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid sequence molecules and nucleotide sequences of the invention can be considered to be “isolated” as defined above.

Thus, an “isolated nucleic acid molecule” or “isolated nucleotide sequence” is a nucleic acid molecule or nucleotide sequence that is not immediately contiguous with nucleotide sequences with which it is immediately contiguous (one on the 5′ end and one on the 3′ end) in the naturally occurring genome of the organism from which it is derived. Accordingly, in one embodiment, an isolated nucleic acid includes some or all of the 5′ non-coding (e.g., promoter) sequences that are immediately contiguous to a coding sequence. The term therefore includes, for example, a recombinant nucleic acid sequence that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant nucleic acid sequence that is part of a hybrid nucleic acid molecule encoding an additional polypeptide or peptide sequence. An “isolated nucleic acid molecule” or “isolated nucleotide sequence” can also include a nucleotide sequence derived from and inserted into the same natural, original cell type, but which is present in a non-natural state, e.g., present in a different copy number, and/or under the control of different regulatory sequences than that found in the native state of the nucleic acid molecule.

The term “isolated” can further refer to a nucleic acid molecule, nucleotide sequence, polypeptide, peptide or fragment that is substantially free of cellular material, viral material, and/or culture medium (e.g., when produced by recombinant DNA techniques), or chemical precursors or other chemicals (e.g., when chemically synthesized). Moreover, an “isolated fragment” is a fragment of a nucleic acid molecule, nucleotide sequence or polypeptide that is not naturally occurring as a fragment and would not be found as such in the natural state. “Isolated” does not necessarily mean that the preparation is technically pure (homogeneous), but it is sufficiently pure to provide the polypeptide or nucleic acid in a form in which it can be used for the intended purpose.

In representative embodiments of the invention, an “isolated” nucleic acid molecule, nucleotide sequence, and/or polypeptide is at least about 5%, 10%, 15%, 20%, 25%, 30%, 40%, 50%, 60%, 70%, 75%, 80%, 85%, 90%, 95%, 97%, 98%, 99% pure (w/w) or more. In other embodiments, an “isolated” nucleic acid, nucleotide sequence, and/or polypeptide indicates that at least about a 5-fold, 10-fold, 25-fold, 100-fold, 1000-fold, 10,000-fold, 100,000-fold or more enrichment of the nucleic acid (w/w) is achieved as compared with the starting material.

“Wild-type” nucleotide sequence or amino acid sequence refers to a naturally occurring (“native”) or endogenous nucleotide sequence or amino acid sequence. Thus, for example, a “wild-type mRNA” is an mRNA that is naturally occurring in or endogenous to the organism.

As used herein, the phrases “operably linked,” “operatively linked,” “operatively associated” or “in operative association” and the like, mean that elements of a nucleic acid construct such as an expression cassette or nucleic acid molecule are configured so as to perform their usual function. Thus, regulatory or control sequences (e.g., promoters) operatively associated with a nucleotide sequence are capable of effecting expression of the nucleotide sequence. For example, a promoter is operably linked with a coding sequence or functional RNA when it is capable of affecting the expression of that coding sequence or functional RNA (i.e., the coding sequence or functional RNA is under the transcriptional control of the promoter). Coding sequences in sense or antisense orientation can be operably-linked to regulatory sequences. The control sequences need not be contiguous with the nucleotide sequence of interest, as long as they function to direct the expression thereof. Thus, for example, intervening untranslated, yet transcribed, sequences can be present between a promoter and a coding sequence, and the promoter sequence can still be considered “operably linked” to the coding sequence.

“Primers” as used herein are isolated nucleic acids that are annealed to a complementary target DNA strand by nucleic acid hybridization to form a hybrid between the primer and the target DNA strand, then extended along the target DNA strand by a polymerase, such as DNA polymerase. Primer pairs or sets can be used for amplification of a nucleic acid molecule, for example, by the polymerase chain reaction (PCR) or other nucleic-acid amplification methods.

A “probe” is an isolated nucleic acid molecule that is complementary to a portion of a target nucleic acid molecule and is typically used to detect and/or quantify the target nucleic acid molecule. Thus, in some embodiments, a probe can be an isolated nucleic acid molecule to which is attached a detectable moiety or reporter molecule, such as a radioactive isotope, ligand, chemiluminescent agent, fluorescence agent or enzyme. Probes according to the present invention can include not only deoxyribonucleic or ribonucleic acids but also polyamides and other probe materials that bind specifically to a target nucleic acid sequence and can be used to detect the presence of and/or quantify the amount of, that target nucleic acid sequence.

A TaqMan probe is designed such that it anneals within a DNA region amplified by a specific set of primers. As the Taq polymerase extends the primer and synthesizes the nascent strand from a single-strand template from 3′ to 5′ of the complementary strand, the 5′ to 3′ exonuclease of the polymerase extends the nascent strand through the probe and consequently degrades the probe that has annealed to the template. Degradation of the probe releases the fluorophore from it and breaks the close proximity to the quencher, thus relieving the quenching effect and allowing fluorescence of the fluorophore. Hence. fluorescence detected in the quantitative PCR thermal cycler is directly proportional to the fluorophore released and the amount of DNA template present in the PCR.

Primers and probes are generally between 5 and 100 nucleotides or more in length. In some embodiments, primers and probes can be at least 20 nucleotides or more in length, or at least 25 nucleotides or more, or at least 30 nucleotides or more in length. Such primers and probes hybridize specifically to a target sequence under optimum hybridization conditions as are known in the art. Primers and probes according to the present invention may have complete sequence complementarity with the target sequence, although probes differing from the target sequence and which retain the ability to hybridize to target sequences may be designed by conventional methods according to the invention.

Methods for preparing and using probes and primers are described, for example, in Molecular Cloning: A Laboratory Manual. 2nd ed., vol. 1-3, ed. Sambrook et al., Cold Spring Harbor Laboratory Press, Cold Spring Harbor, N.Y., 1989. PCR-primer pairs can be derived from a known sequence, for example, by using computer programs intended for that purpose.

The polymerase chain reaction (PCR) is a technique for “amplifying” a particular piece of DNA. In order to perform PCR, at least a portion of the nucleotide sequence of the DNA molecule to be replicated must be known. In general, primers or short oligonucleotides are used that are complementary (e.g., substantially complementary or fully complementary) to the nucleotide sequence at the 3′ end of each strand of the DNA to be amplified (known sequence). The DNA sample is heated to separate its strands and is mixed with the primers. The primers hybridize to their complementary sequences in the DNA sample. Synthesis begins (5′ to 3′ direction) using the original DNA strand as the template. The reaction mixture must contain all four deoxynucleotide triphosphates (dATP, dCTP, dGTP, dTTP) and a DNA polymerase. Polymerization continues until each newly-synthesized strand has proceeded far enough to contain the sequence recognized by the other primer. Once this occurs, two DNA molecules are created that are identical to the original molecule. These two molecules are heated to separate their strands and the process is repeated. Each cycle doubles the number of DNA molecules. Using automated equipment, each cycle of replication can be completed in less than 5 minutes. After 30 cycles, what began as a single molecule of DNA has been amplified into more than a billion copies (2³⁰=1.02×10⁹).

The oligonucleotides of an oligonucleotide primer pair are complementary to DNA sequences located on opposite DNA strands and flanking the region to be amplified. The annealed primers hybridize to the newly synthesized DNA strands. The first amplification cycle will result in two new DNA strands whose 5′ end is fixed by the position of the oligonucleotide primer but whose 3′ end is variable ('ragged' 3′ ends). The two new strands can serve in turn as templates for synthesis of complementary strands of the desired length (the 5′ ends are defined by the primer and the 3′ ends are fixed because synthesis cannot proceed past the terminus of the opposing primer). After a few cycles, the desired fixed length product begins to predominate.

“Detection kit” as used herein refers to a kit used to detect target DNA from the events of interest in a sample comprising nucleic acid probes and primers of the present invention, which will be processed specifically under optimum conditions to a target DNA sequence, and other materials necessary to enable nucleic acid hybridization and/or amplification methods.

As used herein, the terms “transformed” and “transgenic” refer to any plant, plant cell, callus, plant tissue, or plant part that contains all or part of at least one recombinant (e.g., heterologous) polynucleotide. In some embodiments, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The term “plant part,” as used herein, includes but is not limited to embryos, pollen, ovules, seeds, leaves, stems, shoots, flowers, branches, fruit, kernels, cars, cobs, husks, stalks, roots, root tips, anthers, plant cells including plant cells that are intact in plants and/or parts of plants, plant protoplasts, plant tissues, plant cell tissue cultures, plant calli, plant clumps, and the like. As used herein, “shoot” refers to the above ground parts including the leaves and stems. Further, as used herein, “plant cell” refers to a structural and physiological unit of the plant, which comprises a cell wall and also may refer to a protoplast. A plant cell of the present invention can be in the form of an isolated single cell or can be a cultured cell or can be a part of a higher-organized unit such as, for example, a plant tissue or a plant organ.

The term “transformation” as used herein refers to the transfer of a nucleic acid molecule into the genome of a host cell, resulting in genetically stable inheritance. In some embodiments, the introduction into a plant, plant part and/or plant cell is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethylene glycol-mediated transformation, protoplast transformation, or any other electrical, chemical, physical and/or biological mechanism that results in the introduction of nucleic acid into the plant, plant part and/or cell thereof, or any combination thereof.

Procedures for transforming plants are well known and routine in the art and are described throughout the literature. Non-limiting examples of methods for transformation of plants include transformation via bacterial-mediated nucleic acid delivery (e.g., via bacteria from the genus Agrobacterium), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation., sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell Mol Biol Lett 7:849-858 (2002)).

Agrobacterium-mediated transformation is a commonly used method for transforming plants because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. 1993, Plant Cell 5:159-169). The transfer of the recombinant binary vector to Agrobacterium can be accomplished by a tri-parental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen and Willmitzer 1988, Nucleic Acids Res 16:9877).

Transformation of a plant by recombinant Agrobacterium usually involves co-cultivation of the Agrobacterium with explants from the plant and follows methods well known in the art. Transformed tissue is typically regenerated on selection medium carrying an antibiotic or herbicide resistance marker between the binary plasmid T-DNA borders.

Another method for transforming plants, plant parts and plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacteria or a bacteriophage, each containing one or more nucleic acids sought to be introduced) also can be propelled into plant tissue.

Thus, in particular embodiments of the present invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

The term “introducing” or “introduce” in the context of a plant cell, plant and/or plant part means contacting a nucleic acid molecule with the plant, plant part, and/or plant cell in such a manner that the nucleic acid molecule gains access to the interior of the plant cell and/or a cell of the plant and/or plant part. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into plant cells in a single transformation event, in separate transformation events, or, e.g., as part of a breeding protocol. Thus, the term “transformation” as used herein refers to the introduction of a heterologous nucleic acid into a cell. Transformation of a cell may be stable or transient. Thus, a transgenic plant cell, plant and/or plant part of the invention can be stably transformed or transiently transformed.

As used herein, “stably introducing.” “stably introduced.” “stable transformation” or “stably transformed” in the context of a polynucleotide introduced into a cell, means that the introduced polynucleotide is stably integrated into the genome of the cell, and thus the cell is stably transformed with the polynucleotide. As such, the integrated polynucleotide is capable of being inherited by the progeny thereof, more particularly, by the progeny of multiple successive generations. “Genome” as used herein includes the nuclear and/or plastid genome, and therefore includes integration of a polynucleotide into, for example, the chloroplast genome. Stable transformation as used herein can also refer to a polynucleotide that is maintained extrachromasomally, for example, as a minichromosome.

Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

“Primary transformant” and “T0 generation” refer to transgenic plants that are of the same genetic generation as the tissue that was initially transformed (i.e., not having gone through meiosis and fertilization since transformation). “Secondary transformants” and the “T1, T2, T3, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The “transformation and regeneration process” refers to the process of stably introducing a transgene into a plant cell and regenerating a plant from the transgenic plant cell. As used herein, transformation and regeneration includes the selection process, whereby a transgene comprises a selectable marker and the transformed cell has incorporated and expressed the transgene, such that the transformed cell will survive and developmentally flourish in the presence of the selection agent. “Regeneration” refers to growing a whole plant from a plant cell, a group of plant cells, or a plant piece such as from a protoplast, callus, or tissue part. Regeneration methods from a transformed plant cell, for example a transformed maize cell, are well-known in the art.

“Transient transformation” in the context of a polynucleotide means that a polynucleotide is introduced into the cell and does not integrate into the genome of the cell.

Transient transformation may be detected by, for example, an enzyme-linked immunosorbent assay (ELISA) or Western blot, which can detect the presence of a peptide or polypeptide encoded by one or more nucleic acid molecules introduced into an organism. Stable transformation of a cell can be detected by, for example, a Southern blot hybridization assay of genomic DNA of the cell with nucleic acid sequences which specifically hybridize with a nucleotide sequence of a nucleic acid molecule introduced into an organism (e.g., a plant). Stable transformation of a cell can be detected by, for example, a Northern blot hybridization assay of RNA of the cell with nucleic acid sequences which specifically hybridize with a nucleotide sequence of a nucleic acid molecule introduced into a plant or other organism. Stable transformation of a cell can also be detected by, e.g., a polymerase chain reaction (PCR) or other amplification reaction as are well known in the art, employing specific primer sequences that hybridize with target sequence(s) of a nucleic acid molecule, resulting in amplification of the target sequence(s), which can be detected according to standard methods. Transformation can also be detected by direct sequencing and/or hybridization protocols well known in the art.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 70%, least about 75%, at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues to about 150 residues in length. Thus, in some embodiments of this invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In a further embodiment, the sequences are substantially identical over the entire length of the coding regions. Furthermore, in representative embodiments, substantially identical nucleotide or protein sequences perform substantially the same function (e.g., conferring increased resistance to a nematode plant parasite, reducing the growth of a nematode plant parasite, reducing cyst development).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e.,, the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity.” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W. T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially identical when the two sequences hybridize to each other under stringent conditions. In some representative embodiments, two nucleotide sequences considered to be substantially identical hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the present invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2×SSC. 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1×SSC. 0.1% SDS at 50° C. or in 7% sodium dodecyl sulfate (SDS), 0.5 M NaPO₄, 1 mM EDTA at 50° C. with washing in 0.5×SSC. 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1×SSC. 0.1% SDS at 50° C., or in 7% sodium dodecyl sulfate (SDS), 0.5 M NaPO₄, 1 mM EDTA at 50° C. with washing in 0.1×SSC, 0.1% SDS at 65° C.

In the current state of the art, introducing a DNA of interest into a maize cell is typically done using Agrobacterium-mediated transformation or biolistic bombardment. These methods rely on the random insertion of the DNA of interest, such as a transgene, into the maize genome. The expression of foreign genes in plants can be influenced by their chromosomal position, for example due to chromatin structure and/or the proximity of transcriptional regulation elements close to the integration site (See for example, Weising et al., 1988, “Foreign Genes in Plants,” Ann. Rev. Genet. 22:421-477). A high-quality transgenic event is preferred to not be in a promoter or gene region of the genome. A high-quality transgenic event also must not have negative effects on the agronomic performance of the transgenic plant. Additionally, a high-quality transgenic event is the result of a single, intact, transgene insertion, with little or no transgene rearrangement, and without contamination by extraneous heterologous DNA, such as DNA from the backbone of a vector used during the transformation process. A high-quality transgenic event also is preferred to lack introduced ORFs, which potentially may be expressed in the transgenic plant.

It is common to produce hundreds of different events and screen those events for a single event that has desired molecular qualities and transgene expression levels and patterns for commercial purposes. The identified event which satisfies all criteria required for a high-quality event which may be used for commercial purposes is considered an elite event. The elite event is characterized by its exact genomic location, as it is that location which is responsible for the molecular qualities, transgene expression levels, and agronomic performance of the event. The effort required to identify an elite event is on the scale of a large research program. Therefore, there is a desire in the art for novel, more efficient methods of introducing a DNA of interest into a maize cell to produce a high-quality transgenic event.

The recent development of methods and compositions which make targeted genomic insertion relatively less labor intensive provide a critical piece for the technical solution for improved methods of insertion of heterologous nucleic acids into a genome of interest. The present invention includes ideal genomic locations, or loci, for methods for targeted genomic insertion. Successful targeted insertion into any one of these genomic loci can produce a high-quality transgenic event.

Ideal target sites for genomic modifications, in particular for targeted insertion of a DNA of interest into a maize genome, must satisfy a number of criteria. These desirable genomic target sites may also be referred to as “ideal genomic loci”, “target genomic loci”, “safe harbor sites”, or “safe harbors”, and refer to regions of contiguous nucleic acids in the genome that are the selected or preferred site for insertion of a nucleotide sequence of interest (for example, a donor sequence) into the genome. Based on the current knowledge of plant genome organization, gene structure and expression, DNA recombination, genome engineering and GM product regulatory requirements, the following artificially defined criteria were used to identify ideal genomic loci that are suitable for targeted integration and stable expression: (1) regions that contain mostly unique sequences and may be suitable for targeted integration mediated by homologous recombination; (2) regions that are not part of a known functional gene, including those encoding for miRNAs; ideally, these regions should be at least 2 Kb upstream of any known open reading frame or 1 Kb downstream from the 3′-untranslated region (3′-UTR) of a gene, so that integration of a DNA of interest may not interrupt endogenous gene sequences or affect function of neighboring endogenous genes; (3) regions that are not close to heterochromatic regions with highly repetitive sequences such as pericentromeric regions that may result in unstable expression of transgenes or potential silencing of inserted transgenes; (4) regions that do not contain known cis-acting elements such as enhancers or repressors so that transgene expression pattern and level is not altered unexpectedly when inserted; (5) regions that have empirical data showing good transgene expression, if possible. An example of a target maize genomic locus may comprise a nucleic acid sequence of at least 10 nucleotides, at least 20, 25, 30, 40, 50, 60, 70, 75, 80, 90,100, 150, 200, 250, 300, 250, 400, 450, 500, 600, 700, 800, 900, 1000, 1500, 2000, 2500, 3000, 3500, 4000, 4500, or at least 5000 nucleotides, and have at least 70%, at least 75%, at least 80%, at least 85%, at least 90%, at least 93%, at least 95%, at least 96%, at least 97%, at least 98%, at least 99%, or 100% identity to at least 10 nucleotides, at least 20, 25, 30, 40, 50, 60, 70, 75, 80, 90, 100, 150, 200, 250, 300, 250, 400, 450, 500, 600, 700, 800, 900, 1000, 1500, 2000, 2500, 2750, 2900, or at least 3000 nucleotides of any one of SEQ ID NO: 1-71 or a complement thereof.

Targeting insertion of a DNA of interest into safe harbors identified by this criteria is likely to result in a transgenic plant which has minimal issues regarding stable expression levels. Targeting the DNA of interest to a safe harbor also eliminates significant screening required to identify events produced by random genomic insertion which satisfy the “safe harbor” criteria. Additionally, the identification of safe harbor sites which are highly conserved in more than one maize variety indicates that the safe harbor in one maize variety is likely to be a safe harbor in a different maize variety. This is important for introgression of the DNA of interest into multiple varieties for commercial agricultural use.

As used herein a “DNA of interest”, “nucleic acid of interest”, or “nucleotide sequence of interest”, is defined as a nucleic acid/DNA sequence that has been selected for site directed, targeted insertion into the maize genome. A nucleic acid of interest can be of any length, for example between 2 and 50,000 nucleotides in length (or any integer value there between or there above), preferably between about 1,000 and 5,000 nucleotides in length (or any integer value there between). A DNA of interest may comprise one or more gene expression cassettes that further comprise actively transcribed and/or translated gene sequences. Conversely, the DNA of interest may comprise a polynucleotide sequence which does not comprise a functional gene expression cassette or an entire gene (e.g., may simply comprise regulatory sequences such as a promoter), or may not contain any identifiable gene expression elements or any actively transcribed gene sequence. The DNA of interest may optionally contain an analytical domain, such as a domain that may contain specifically designed restriction enzyme sites, zinc finger binding sites, engineered landing pads, or engineered transgene integration platforms. Upon insertion of the nucleic acid of interest into the maize genome, the inserted sequences may be referred to, for example, as the “inserted DNA of interest”. Further, the nucleic acid of interest can be DNA or RNA, can be linear or circular, and can be single-stranded or double-stranded. It can be delivered to the cell as naked nucleic acid, as a complex with one or more delivery agents (e.g., liposomes, poloxamers, T-strand encapsulated with proteins, a Feldan Shuttle (U.S. Patent Publication No. 20160298078, herein incorporated by reference) etc.,) or contained in a bacterial or viral delivery vehicle, such as, for example, Agrobacterium tumefaciens or an adenovirus.

A DNA of interest may further comprise a “gene of interest”. “Gene of interest” refers to any gene which, when transferred to a plant, confers upon the plant a desired characteristic such as antibiotic resistance, virus resistance, insect resistance, disease resistance, or resistance to other pests, herbicide tolerance, improved nutritional value, improved performance in an industrial process or altered reproductive capability. The “gene of interest” may also be one that is transferred to plants for the production of commercially valuable enzymes or metabolites in the plant.

A “donor”, “donor molecule”, or “donor sequence” as used herein comprises a DNA of interest. The donor molecule may further comprise homologous arms or other nucleic acid sequences useful for recombination of the nucleic acid of interest into the target site of the host genome. The donor sequence may comprise one or more transgenes, expression cassettes, or other nucleotide sequences of interest. A donor molecule may be single stranded, partially double-stranded, or double-stranded. The donor molecule may be a natural or a modified polynucleotide, a RNA-DNA chimera, or a DNA fragment, either single—or at least partially double-stranded, or a fully double-stranded DNA molecule, or a PGR amplified ssDNA or at least partially dsDNA fragment. In some embodiments, the donor DNA molecule is part of a circularized DNA molecule. A fully double-stranded donor DNA is advantageous since it might provide an increased stability, since dsDNA fragments are generally more resistant than ssDNA to nuclease degradation. In some embodiments, the donor polynucleotide molecule can comprise at least about 100, 150, 200, 250, 300, 250, 400, 450, 500, 600, 700, 800, 900, 1000, 1500, 2000, 2500, 3000, 3500, 4000, 4500, 5000, 7500, 10000, 15,000 or at least about 20,000 nucleotides, including any value within this range not explicitly recited herein. In some embodiments, the donor DNA molecule comprises a heterologous nucleic acid sequence. In some embodiments, the donor DNA molecule comprises at least one expression cassette. In some embodiments, the donor DNA molecule may comprise a transgene. In some embodiments, the donor DNA molecule comprises an allelic modification of a gene which is native to the target genome. The allelic modification may comprise at least one nucleotide insertion, at least one nucleotide deletion, and/or at least one nucleotide substitution. In some embodiments, the allelic modification may comprise an INDEL. In some embodiments, the donor DNA molecule comprises at least 50 nucleotides, at least 60, 70, 80, 90, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or at least 20,000 nucleotides, which have at least 70%, at least 75%, at least 80%, at least 85%, at least 90%, at least 93%, at least 95%, at least 96%, at least 97%, at least 98%, at least 99%, or 100% identity to at least 50 contiguous nucleotides, 60, 70, 80, 90, 100, 150, 200, 250, 300, 250, 400, 450, 500, 600, 700, 800, 900, 1000, 1500, 2000, 2500, 2750, 2900, or at least 3000 contiguous nucleotides of SEQ ID NO: 1-71 or a complement thereof, and wherein the donor molecule further comprises a DNA of interest. In some embodiments, the donor DNA molecule comprises at least 100 contiguous nucleotides at least 90% identical to a genomic nucleic acid sequence, and optionally may further comprise a heterologous nucleic acid sequence such as a transgene.

A “homologous” nucleic acid sequence is a nucleic acid sequence naturally associated with a host cell into which it is introduced. A homologous nucleic acid sequence can also be a nucleic acid sequence that is naturally associated with other nucleic acid sequences that may be present, e.g., in a nucleic acid construct. As one nonlimiting example, a promoter may be present in a nucleic acid construct in combination with one or more regulatory elements and/or coding sequences that naturally occur in association with that particular promoter, i.e., they are homologous to the promoter.

In some instances “homologous” may be used to refer to the relationship of a first gene to a second gene by descent from a common ancestral DNA sequence. In such instances, the term “homolog” indicates a relationship between genes separated by the event of speciation (or an “ortholog”) or to the relationship between genes separated by the event of genetic duplication (or a “paralog”). In other instances “homologous” may be used to refer to the level of sequence identity between one or more polynucleotide sequences, in such instances the one or more polynucelotide sequences do not necessarily descend from a common ancestral DNA sequence. Those with skill in the art are aware of the interchangeably of the term “homologous” and appreciate the proper application of the term.

Targeted genomic insertion methods of the invention require a site-directed nuclease and a nucleic acid molecule comprising the DNA of insertion as well as at least one homologous arm which is important for homologous recombination of the nucleic acid molecule into the target genomic locus. The target genomic locus comprises a nuclease cleavage site, which may be a targeted site for a site-directed nuclease.

A “nuclease cleavage site” or “genomic nuclease cleavage site” is a region of nucleotides that comprise a nuclease cleavage sequence that is recognized by a specific nuclease, which acts to cleave the nucleotide sequence of the DNA of the genomic nuclease cleavage site in one or both strands. Such cleavage by the nuclease enzyme initiates DNA repair mechanisms within the cell, which establishes an environment for homologous recombination to occur. In the methods herein wherein a first nucleic acid molecule comprises, for example, at least about 100 contiguous nucleotides having, for example, at least 90% identity with a target site in the genome of the cell, the first nucleic acid molecule may be integrated into the genome of the cell via homologous recombination, thereby integrating the one or more DNAs of interest into the genome of the cell.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends.

The nuclease of the methods of the invention may be engineered to target the nucleic acid sequence of the genomic nuclease cleavage site. In some embodiments, the genomic nuclease cleavage site may be unique to the maize genome. In other embodiments, the genomic nuclease cleavage site may occur infrequently in the maize genome. “Infrequently” may be less than 500 occurrences, less than 400, 300, 200, 100, 90, 80, 70, 60, 50, 40, 30, 20, 10, 5, or less than 3 occurrences in the maize genome.

In some embodiments, the nuclease in the methods of the invention may be a site specific nuclease, such a meganuclease, a zinc finger nuclease, a TALEN, or a CRISPR-associated nuclease. In some embodiments, the meganuclease is a homing endonuclease, for example I-SceI or I-CreI. In some embodiments, the CRISPR-associated nuclease is a Cas9, CpfI, or dCas9 (“dead” Cas9) or dCpf1 (“dead” Cpf1). For the “dead” CRISPR-associated nuclease, the nuclease activity of the RNA binding protein is inactivated.

In some embodiments, the site specific nuclease is a TALEN or a zinc finger nuclease. In some embodiments, the TALEN or zinc finger nuclease may be chimeric. The TALEN and/or zinc finger nuclease may bind to the maize genomic target site and cleave the maize genomic target site, where upon the DNA of interest integrates within or proximal to the maize genomic target site. In an embodiment, integration of the DNA of interest occurs within the maize genomic target site may result in rearrangements. In some embodiments, the rearrangements may comprise deletions, insertions, inversions, and repeats. In one embodiment, integration of the DNA of interest may occur proximal to the maize genomic target site. According to an aspect of the embodiment, the integration site of the DNA of interest is proximal to the target maize genomic locu, and may integrate within 1.5 Kb, 1.25 Kb, 1.0 Kb, 0.75 Kb, 0.5 Kb, 0.25 Kb, 0.15 Kb, 0.10 Kb, 50 bp, 25 bp, 10 bp, or within 5 bp to the maize genomic target site. Insertion within a genomic region proximal to the maize genomic target site is known in the art, see for example for zinc finger nucleases US Patent Pub No. 2010/0257 638 A1 (herein incorporated by reference in its entirety). As used herein, the terms “adjacent” or “adjacent to” with regard to one or more nucleotide sequences of this invention means immediately next to (e.g., with no intervening sequence) or separated by from about 1 base to about 1,000 bases (e.g., 1, 2, 3, 4, 5, 6, 7, 8, 9, 10, 15, 20, 30, 40, 50, 100, 200, 500, or 1,000 bases), including any values included within this range but not explicitly recited herein.

Zinc finger, meganuclease, and TALE DNA binding domains can be “engineered” to bind to a predetermined nucleotide sequence, for example via engineering (altering one or more amino acids) of the recognition helix region of a naturally occurring zinc finger. Similarly, TALENs and meganucleases can be “engineered” to bind to a predetermined nucleotide sequence, for example by engineering of the amino acids involved in DNA binding (for example, the repeat variable diresidue or RVD region in a TALEN). Therefore, engineered DNA binding proteins (zinc fingers, meganucleases, or TALENs) are proteins that are non-naturally occurring.

Non-limiting examples of methods for engineering DNA-binding proteins are design and selection. A designed DNA binding protein is a protein not occurring in nature whose design/composition results primarily from rational criteria. Rational criteria for design include application of substitution rules and computerized algorithms for processing information. Such rational criteria can be applied for the design of zinc fingers. TALENs, meganucleases, or CRISPR-associated nucleases. See, for example, U.S. Pat. Nos. 6,140,081, 6,453,242, 6,534,261; see also WO 98/53058; WO98/53059; WO 98/53060; WO 02/016536 and WO03/016496 and U.S. Publication Nos. 20110301073, 20110239315 and 20119145940. A “selected” zinc finger protein, CRISPR-associated nuclease, meganuclease, or TALEN is a protein not found in nature whose production results primarily from an empirical process such as phage display, interaction trap or hybrid selection. See e.g., WO 96/06166; WO 98/53057; WO 98/54311; WO00/27878; WO 01/60970 WO 01/88197 and WO 02/099084 and U.S. Publication Nos. 20110301073, 20110239315 and 20119145940.

In some embodiments, the DNA-binding domain is an engineered domain from a TAL effector derived from the plant pathogen Xanthomonas (see, Miller et al. (2011) Nature Biotechnology 29 (2): 143-8; Boch et al. (2009) Science 29 Oct. 2009 (10.1 126/science.117881) and Moscou and Bogdanove, (2009) Science 29 Oct. 2009 (10.1126/science.1178817; and U.S. Patent Publication Nos. 20110239315, 20110145940 and 20110301073).

In some embodiments, the DNA-binding domain may be associated with a cleavage (nuclease) domain. For example, homing endonucleases may be modified in their DNA-binding specificity while retaining nuclease function. In addition, zinc finger proteins may also be fused to a cleavage domain to form a zinc finger nuclease. The cleavage domain portion of the fusion proteins disclosed herein can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). Non limiting examples of homing endonucleases and meganucleases include I-SceI, I-Ceul, PI-Pspl, PI-See, I-SceIV, I-Csml, I-PanI, I-Scell, I-PpoI, I-SceIII, I-CreI-TevI, I-TevII and I-TevIII. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. One or more of these enzymes (or functional fragments thereof) can be used as a source of cleavage domains and cleavage half-domains.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites proximal to the recognition site and have separable binding and cleavage domains. For example, the Type IIS enzyme Fokl catalyzes double stranded cleavage of DNA at 9 nucleotides from its recognition site on one strand and 13 nucleotides from its recognition site on the other. See, for example, U.S. Pat. Nos. 5,356,802, 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins may comprise the cleavage domain (or cleavage half domain) from at least one Type IIS restriction enzyme and one or more zinc finger binding domains, which may or may not be engineered. An exemplary Type IIS restriction enzyme, whose cleavage domain is separable from the binding domain, is Fokl. This particular enzyme is active as a dimer (Bitinaite et al. (1998) Proc. Natl. Acad. Sci. USA 95:10,570-10,575). Accordingly, for the purposes of the present disclosure, the portion of the Fokl enzyme used in the disclosed fusion proteins is considered a cleavage half-domain. A cleavage domain or cleavage half-domain can be any portion of a protein that retains cleavage activity, or that retains the ability to multimerize (e.g., dimerize) to form a functional cleavage domain. Exemplary Type IIS restriction enzymes are described in International Publication WO 2007 1014275, incorporated by reference herein in its entirety. To enhance cleavage specificity, cleavage domains may also be modified. Non-limiting examples of modified cleavage half-domains are described in detail in WO 2007/014275, incorporated by reference in its entirety herein. In certain embodiments, the cleavage domain comprises an engineered cleavage half-domain (also referred to as dimerization domain mutants) that minimize or prevent homodimerization. Such embodiments are known to those of skill the art and described for example in U.S. Patent Publication Nos.20050064474; 20060188987; 20070305346 and 20080131962, the disclosures of all of which are incorporated by reference in their entireties herein. Amino acid residues at positions 446, 447, 479, 483, 484, 486, 487, 490, 491, 496,498, 499, 500, 531, 534, 537, and 538 of FokI are all targets for influencing dimerization of the Fokl cleavage half-domains.

The term “CRISPR-associated protein”, “Cas protein”, “CRIPSR-associated nuclease” or “Cas nuclease” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the Cas mutant or Cas variant substantially retains the nuclease activity of the Cas protein, such as a Cas9 variant described herein which is operably linked to a nuclear localization signal (NLS) derived from a plant. In certain embodiments, the Cas nuclease is mutated such that one or both nuclease domains are inactive, such as, for example, a catalytically dead Cas9 referred to as dCas9, which is still able to target to a specific genomic location but has no endonuclease activity (Qi et al., 2013, Cell. 152:1173-1183, hereby incorporated within). In some embodiments, the Cas nuclease is mutated so that it lacks some or all of the nuclease activity of its wild-type counterpart. The Cas protein may be Cas9, Cpf1 (Zetsche et al., 2015, Cell, 163:759-771, hereby incorporated within) or another CRISPR-associated nuclease.

As used herein, the term “guide RNA” or “gRNA” generally refers to an RNA molecule (or a group of RNA molecules collectively) that can bind to a Cas protein and aid in targeting the Cas protein to a specific location within a target polynucleotide (e.g., a DNA). A guide RNA can comprise a crRNA segment and/or a tracrRNA. The present invention further provides a guide RNA of the invention comprising a tracrRNA, wherein the tracrRNA comprises a nucleic acid sequence that is capable of binding to protein. A guide RNA of the invention also encompasses an engineered chimeric single guide RNA (sgRNA), where the crRNA segment and the tracrRNA segment are located in the same RNA molecule. A “guide RNA” also encompasses, collectively, a group of two (“dual guide RNA”) or more RNA molecules, where the crRNA segment and the tracrRNA segment are located in separate RNA molecules. As used herein, the term “crRNA” or “crRNA segment” refers to an RNA molecule or portion thereof that includes a polynucleotide targeting guide sequence, a stem sequence, and, optionally, a 5′-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention further provides an engineered guide RNA comprising a chimeric crRNA segment comprising a guide RNA sequence capable of hybridizing to a genomic target sequence, a CRISPR repeat sequence and a bait RNA segment capable of hybridizing to a donor DNA molecule. In some embodiments, the guide RNA, either as a sgRNA or as two or more RNA molecules, does not contain a tracrRNA, as it is known in the art that some CRISPR-associated nucleases, such as Cpf1, do not require a tracrRNA for its RNA-mediated endonuclease activity (Qi et al., 2013).

The present invention also provides methods which include a nucleic acid molecule comprising a nucleic acid sequence encoding a guide RNA of the invention. The nucleic acid molecule may be a DNA or an RNA molecule. In some embodiments, the nucleic acid molecule is circularized. In other embodiments, the nucleic acid molecule is linear. In some embodiments, the nucleic acid molecule is single stranded, partially double-stranded, or double-stranded. In some embodiments, the nucleic acid molecule is complexed with at least one polypeptide. In some embodiments, the polypeptide is a carrier protein for mediating delivery of, for example, the guide RNA, a nuclease, and optionally a donor molecule. In some embodiments, the polypeptide is a Feldan Shuttle (U.S. Patent Publication No. 20160298078, herein incorporated by reference). The nucleic acid molecule may comprise an expression cassette capable of driving the expression of the guide RNA. The nucleic acid molecule may further comprise additional expression cassettes, capable of expressing, for example, a nuclease such as a CRISPR-associated nuclease.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the nucleotide sequence that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide. For HR-directed integration, the donor molecule contains at least 1, typically at least 2 regions of homology to the genome (“homology arms”) of least 50-100 base pairs in length. See, e.g., U.S. Pat. No. 9,045,763. In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break for HR mediated integration or having no homology to the nucleotide sequence in the region of the break for NHEJ mediated integration, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another. In any of the methods described herein, additional zinc-finger proteins, meganucleases, CRISPR-associated nucleases, or TALENs can be used for additional double-stranded cleavage of additional target sites within the cell.

Accordingly, the present invention provides a maize recombinant polynucleotide, wherein the recombinant polynucleotide comprises a nucleic acid sequence of at least 50 nucleotides, at least 60, 70, 80, 90, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or at least 20,000 nucleotides, and has at least 70%, at least 75%, at least 80%, at least 85%, at least 90%, at least 93%, at least 95%, at least 96%, at least 97%, at least 98%, at least 99%, or 100% identity to at least 50 contiguous nucleotides, 60, 70, 80, 90, 100, 150, 200, 250, 300, 250, 400, 450, 500, 600, 700, 800, 900, 1000, 1500, 2000, 2500, 2750, 2900, or at least 3000 contiguous nucleotides of SEQ ID NO: 1-71 or a complement thereof, and wherein the recombinant polynucleotide further comprises a DNA of interest, wherein the DNA of interest is inserted into the nucleic acid sequence to produce said recombinant polynucleotide.

In further embodiments, the recombinant polynucleotide comprises a DNA of interest inserted proximal to a nuclease cleavage site within the recombinant polynucleotide.

In some embodiments, the recombinant polynucleotide comprises a DNA of interest which is a transgene. In some embodiments, the DNA of interest comprises at least one gene of interest. In some embodiments, the DNA of interest comprises at least one expression cassette. In some embodiments, the DNA of interest comprises two or more expression cassettes. In some embodiments, the DNA of interest does not encode a polypeptide. In some embodiments, the DNA of interest comprises regulatory sequences.

In some embodiments, the recombinant polynucleotide comprises a nucleic acid sequence of at least 50 nucleotides, at least 60, 70, 80, 90, 100, 150, 200, 250, 300, 250, 400, 450, 500, 600, 700, 800, 900, 1000, 1500, 2000, 2500, 3000, 3500, 4000, 4500, 5000, 7500, 10000, 15,000 or at least 20,000 nucleotides, and has at least 70%, at least 75%, at least 80%, at least 85%, at least 90%, at least 93%, at least 95%, at least 96%, at least 97%, at least 98%, at least 99%, or 100% identity to at least 50 contiguous nucleotides, at least 60, 70, 80, 90, 100, 150, 200, 250, 300, 250, 400, 450, 500, 600, 700, 800, 900, 1000, 1500, 2000, 2500, 2750, 2900, or at least 3000 contiguous nucleotides of SEQ ID NO: 1, 8, 9, 13, 21, 22, 23, 31, 37, 39, 40, 44, 46, 55, 56, 57, 62-71, or a complement thereof.

The present invention also provides a maize plant, plant part, or plant cell comprising the recombinant polynucleotide described above.

In another embodiment, the present invention provides a method of integrating a DNA of interest into a target maize genomic locus in a maize genome, wherein the target maize genomic locus comprises a nucleic acid sequence of at least 50, at least 60, at least 70, at least 80, at least 90, at least 100, at least 110, at least 120, at least 130, at least 140, at least 150, 200, 250, 300, 250, 400, 450, 500, 600, 700, 800, 900, 1000, 1500, 2000, 2500, 2750, 2900, or at least 3000 contiguous nucleotides having at least 70%, at least 75%, at least 80%, at least 85%, at least 90%, at least 93%, at least 95%, at least 96%, at least 97%, at least 98%, at least 99%, or 100% identity to at least 50 contiguous nucleotides, at least 60, at least 70, at least 80, at least 90, at least 100, at least 110, at least 120, at least 130, at least 140, at least 150, 200, 250, 300, 250, 400, 450, 500, 600, 700, 800, 900, 1000, 1500, 2000, 2500, 2750, 2900, or at least 3000 contiguous nucleotides of SEQ ID NO: 1 through SEQ ID NO: 71, or a complement thereof, comprising introducing into a maize cell: (a) a first nucleic acid molecule comprising at least 50, at least 60, at least 70, at least 80, at least 90, at least 100, at least 110, at least 120, at least 130, at least 140, or at least 150 contiguous nucleotides, wherein said contiguous nucleotides have at least 70%, at least 75%, at least 80%, at least 85%, at least 90%, at least 93%, at least 95%, at least 96%, at least 97%, at least 98%, at least 99%, or 100% identity to at least a 50, at least 60, at least 70, at least 80, at least 90, at least 100, at least 110, at least 120, at least 130, at least 140, or at least a 150 contiguous nucleotides of SEQ ID NO: 1 through SEQ ID NO: 71, or a complement thereof, and further comprising a DNA of interest; and (b) a second nucleic acid molecule comprising a nucleotide sequence encoding a nuclease for site-directed cleavage at a genomic nuclease cleavage site of, adjacent to, or proximal to the genomic nucleotide sequence of SEQ ID NO: 1 through SEQ ID NO: 71, or a complement thereof, that corresponds to the contiguous nucleotides of (a), under conditions wherein expression of the second nucleic acid molecule can occur to produce the nuclease and the nuclease can cleave the nucleotide sequence at the genomic nuclease cleavage site, whereby the DNA of interest is integrated at the genomic nuclease target cleavage site in the maize genome.

In some embodiments of the above method, the first nucleic acid molecule can comprise at least about 100, 150, 200, 250, 300, 250, 400, 450, 500, 600, 700, 800, 900, 1000, 1500, 2000, 2500, 3000, 3500, 4000, 4500, 5000, 7500, 10000, 15,000 or 20,000 nucleotides, including any value within this range not explicitly recited herein.

In some embodiments, the first nucleic acid molecule and the second nucleic acid molecule can be introduced into the maize cell by biolistic nucleic acid delivery, via an Agrobacterium, by co-transformation, and/or with a T-DNA vector in any combination and/or order.

In some embodiments, the first nucleic acid molecule and the second nucleic acid molecule can be present on a single nucleic acid construct and in some embodiments, the first nucleic acid molecule and the second nucleic acid molecule can be present on separate nucleic acid constructs.

In some embodiments, the first nucleic acid molecule and/or the second nucleic acid molecule can be transiently expressed in the maize cell. In some embodiments, the first nucleic acid molecule and/or the second nucleic acid molecule can be stably integrated into the maize genome of the maize cell.

In some embodiments of the methods of the invention, the first nucleic acid molecule is a donor molecule. In some embodiments, the DNA of interest comprises at least one expression cassette. In some embodiments, the DNA of interest comprises a transgene.

In another embodiment, the present invention provides a method of making a maize plant cell comprising a DNA of interest, said method comprising: (a) selecting a target maize genomic locus, wherein the target maize genomic locus comprises a nucleic acid sequence of at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides, and has at least 70%, at least 75%, at least 80%, at least 85%, at least 90%, at least 93%, at least 95%, at least 96%, at least 97%, at least 98%, at least 99%, or 100% identity to at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides of SEQ ID NO: 1-71 or a complement thereof; (b) selecting a site specific nuclease that specifically binds to and cleaves a genomic nuclease cleavage site within said target maize genomic locus; (c) introducing said site specific nuclease and a DNA of interest into the maize plant cell; (d) allowing the DNA of interest to insert into the target maize genomic locus; and (e) selecting maize plant cells that comprise the DNA of interest inserted into the target maize genomic locus.

The site specific nuclease of the method described above may be introduced into the maize plant cell either as a polypeptide or as nucleic acid molecule, which is transcribed and/or translated in the plant cell to produce the site specific nuclease. The site specific nuclease may be transiently expressed in the plant cell. The site specific nuclease may not be expressed in the maize cell, and may only be present in the maize cell as an active nuclease. The site specific nuclease and the DNA of interest may be introduced into the cell simultaneously or not simultaneously.

In some embodiments of the methods of the invention, the genomic nuclease cleavage site is within a target maize genomic locus which comprises a nucleic acid sequence of at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides, and has at least 70%, at least 75%, at least 80%, at least 85%, at least 90%, at least 93%, at least 95%, at least 96%, at least 97%, at least 98%, at least 99%, or 100% identity to at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides of SEQ ID NO: 1, 8, 9, 13, 21, 22, 23, 31, 37, 39, 40, 44, 46, 55, 56, 57, 62-71, or the complement thereof.

In some embodiments of the methods of the invention, the DNA of interest is inserted into the target maize genomic locus via homologous recombination. In other embodiments, the DNA of interest inserted into the target maize genomic locus via non-homologous end-joining. In some embodiments, the DNA of interest and/or the target maize genomic locus are modified during insertion of said DNA of interest into said target maize genomic locus.

In some embodiments of the methods of the invention, two or more DNAs of interest are inserted into two or more target maize genomic loci by any one of the methods described herein.

In some embodiments of the methods of the invention, the DNA of interest comprises at least one expression cassette. In some embodiments, the DNA of interest comprises a transgene. In some embodiments of the methods of the invention, the DNA of interest does not encode for a polypeptide. In some embodiments of the methods of the invention, the DNA of interest encodes for regulatory sequences.

In some embodiments of the methods of the invention, the site specific nuclease is a CRISPR-associated nuclease, a zinc finger nuclease, a TALEN, or a meganuclease, singly or in combination.

In some embodiments of the methods of the invention, the maize plant cell comprising the target maize genomic locus is transgenic, such that it contains a heterologous sequence in its genome prior to the practice of the method.

In some embodiments of the methods of the invention, the site specific nuclease is a CRISPR-associated nuclease, such as Cas9, and the method includes an additional nucleic acid molecule encoding a guide RNA, which is also introduced into the maize cell. The additional nucleic acid molecule may be a DNA molecule that can be expressed in the maize cell to produce the guide RNA, or it may be an RNA molecule, the guide RNA molecule itself, which is introduced into the maize cell.

In some embodiments, methods of integrating a DNA of interest into a target maize genomic locus comprise a nucleic acid molecule which is a donor molecule. The donor molecule may be a donor vector. The donor molecule may be part of the CRISPR-Cas nuclease system. The nucleic acid sequence of the donor molecule may comprise a DNA of interest and also one or more regions that share homology with the targeted genomic locus. Generally, the homologous region(s) of the donor molecule will have at least 50% sequence identity to a genomic sequence with which recombination is desired. In certain embodiments, the homologous region(s) of the nucleic acid of interest shares at least 70%, at least 75%, at least 80%, at least 85%, at least 90%, at least 95%, at least 96%, at least 97%, at least 98%, at least 99%, or 100% sequence identity with sequences located in the targeted genomic locus. However, any value between 1% and 100% sequence identity can be present, depending upon the length of the nucleic acid of interest. A DNA of interest can contain several, discontinuous regions of sequence sharing relatively high sequence identity to cellular chromatin. For example, for targeted insertion of sequences not normally present in a targeted genomic locus, the unique sequences which comprise the DNA of interest can be present in a donor nucleic acid molecule and flanked by regions of sequences that share a relatively high sequence identity to a sequence present in the targeted genomic locus.

In some embodiments, a donor nucleic acid molecule, which comprises a DNA of interest, is introduced into a host cell for targeted insertion into a safe harbor site in the genome, wherein the donor molecule also comprises homologous flanking sequences on one or both ends of the nucleic acid of interest. In such an embodiment, the homologous flanking sequences contain sufficient levels of sequence identity to a maize genomic sequence to support homologous recombination between it and the genomic sequence to which it bears homology. Approximately 25, 50, 100, 200, 500, 750, 1000, 1500, or 2000 nucleotides, with sequence identity of at least 70%, at least 75%, at least 80%, at least 85%, at least 90%, at least 95%, at least 96%, at least 97%, at least 98%, at least 99%, or 100%, between a donor and a genomic sequence will support homologous recombination there between.

In other embodiments of targeted recombination and/or replacement and/or alteration of genomic sequence at the safe harbor, the genomic sequence is altered by homologous recombination with the donor molecule. Such homologous recombination is stimulated by the presence of a double-stranded break in cellular chromatin, if sequences homologous to the region of the break are present. Double-strand breaks in cellular chromatin can also stimulate cellular mechanisms of non-homologous end-joining. In any of the methods described herein, the donor molecule can contain sequences that are homologous, but not identical, to genomic sequences in the safe harbor, thereby stimulating homologous recombination to insert a non-identical sequence in the safe harbor. Thus, in certain embodiments, portions of the donor molecule that are homologous to sequences in the safe harbor exhibit at least 70%. at least 75%, at least 80%, at least 85%, at least 90%, at least 95%, at least 96%, at least 97%, at least 98%, at least 99%, or 100% sequence identity to the genomic sequence that is replaced. In other embodiments, the homology between the donor and genomic sequence is higher than 99%, for example if only 1nucleotide differs as between donor and genomic sequences of over 100 contiguous base pairs. In certain cases, a non-homologous portion of the donor sequence can contain sequences not present in the safe harbor, such that new sequences are introduced into the safe harbor. In these instances, the non-homologous sequence is generally flanked by sequences of 50 to 2,000 base pairs (or any integral value there between) or any number of base pairs greater than 2,000, that are homologous or identical to sequences in the region of interest. In other embodiments, the donor sequence is non-homologous to the region of interest, and is inserted into the genome for example by non-homologous end-joining. In some embodiments, the sequence of the genomic safe harbor site and/or of the nucleic acid sequence of interest is altered by either the homologous recombination or the non-homologous end-joining. Such alterations may be, for example, the insertion and/or deletion of nucleic acids.

The donor molecule comprising the DNA of interest may be a linear or a circularized molecule. In some embodiments, the donor molecule is circularized and is preferably linearized in vivo by a nuclease, for example one or more of the same or different nucleases that are used to introduce the double-stranded break in the genomic safe harbor site. Synchronized cleavage of the chromosome and the donor molecule in the cell may limit donor DNA degradation (as compared to linearization of the donor molecule prior to introduction into the cell). The nuclease target sites used for linearization of the donor preferably do not disrupt the transgene(s) sequence(s). The transgene may be integrated into the genome in the direction expected by simple ligation of the nuclease overhangs (designated “forward” or “AB” orientation) or in the alternate direction (designated “reverse” or “BA” orientation). In certain embodiments, the transgene is integrated following accurate ligation of the donor and chromosome overhangs. In other embodiments, integration of the transgene in either the BA or AB orientation results in insertion and/or deletion of several nucleotides.

In some embodiments of the methods of the invention, the DNA of interest is integrated into the targeted genomic site of the host cell. In the case of multicellular species, such as maize, transgenic cells may be regenerated into maize callus, a maize plant part, or a maize plant. In some embodiments, the transgenic cell may be cultured to produce a transgenic plant, for example, comprising one or more DNA sequences of interest at one or more safe harbor sites in the genome of the transgenic plant.

The present invention further provides a method of producing a maize plant, plant part, or progeny thereof comprising a DNA of interest, such as a transgene, integrated into the genomic nuclease cleavage site in the maize genome, comprising regenerating a maize plant from the maize cell produced by the method described herein. Accordingly, the present invention provides a maize plant, plant part, or progeny thereof comprising the transgene integrated into the genomic nuclease cleavage site in the maize genome, produced by the method of this invention.

In some embodiments of the methods described above, the mutation comprises at least one nucleotide substitution, the deletion of at least one nucleotide, or a combination of substitution, deletion, and/or insertion, such as for example an INDEL.

In some embodiments of the methods described above, the nuclease for site-directed cleavage is a non-engineered nuclease. In some embodiments, the nuclease is an engineered nuclease with programmable cleavage target specificity. In some embodiments, the nuclease is a Cas9.

The present invention additionally provides a method of producing a plant, plant part, or progeny thereof comprising a transgene introduced at a nuclease cleavage site in a genome of a plant cell and lacking integration of a heterologous nucleotide sequence encoding a nuclease for site-directed cleavage of a nucleotide sequence at the nuclease cleavage site in the genome of the plant cell, comprising: a) introducing into the plant cell a nucleic acid molecule comprising a heterologous nucleotide sequence encoding a nuclease for site-directed cleavage of the nucleotide sequence at the nuclease cleavage site in the genome of the plant cell under conditions wherein expression of the nucleic acid molecule occurs transiently to produce the nuclease and the nuclease can cleave the nucleotide sequence at the nuclease cleavage site in the genome of the plant cell, thereby introducing a mutation at the nuclease cleavage site in the genome of the plant cell without integration of the heterologous nucleotide sequence encoding the nuclease into the genome of the plant cell; and b) regenerating a plant, plant part, or progeny thereof from the plant cell of (a). The nuclease cleavage site described above is located within a target genomic locus, which comprises a nucleic acid sequence of at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides, and has at least 70%, at least 75%, at least 80%, at least 85%, at least 90%, at least 93%, at least 95%, at least 96%, at least 97%, at least 98%, at least 99%, or 100% identity to at least 10, 20, 25, 30, 40, 50, 60, 70, 75, 80, 90, 100, 150, 200, 250, 300, 250, 400, 450, 500, 600, 700, 800, 900, 1000, 1500, 2000, 2500, or at least 3,000 contiguous nucleotides of SEQ ID NO: 1-71 or a complement thereof. In another embodiment, the present invention provides for the plant cell produced by the method described above. In a further embodiment, the present invention provides for a plant or plant part regenerated or derived from the plant cell produced by the method described above.

In some embodiments of the method described above, the transgene may comprise at least one, at least two, at least three, at least four, at least five, at least six, at least seven, at least eight, at least nine, at least ten, or ten or more expression cassettes.

In some embodiments of the methods described above, a second nucleic acid molecule comprising a DNA of interest is also introduced into the plant cell. In some embodiments of the methods described above, the first nucleic acid molecule and the second nucleic acid molecule are introduced at the same time, for example by co-transformation, biolistic nucleic acid delivery, or Agrobacterium-mediated transformation. In some embodiments, the first nucleic acid molecule and the second nucleic acid molecule are separate molecules. In some embodiments, a single nucleic acid molecule or construct comprises the first nucleic acid molecule and the second nucleic acid molecule described above.

In some embodiments of the methods described above, the nuclease for site-directed cleavage is a non-engineered nuclease. In some embodiments, the nuclease is an engineered nuclease with programmable cleavage target specificity. In some embodiments, the nuclease is a meganuclease, a TALEN, a zinc finger nuclease, or a CRISPR-associated nuclease, such as Cas9.

The present invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Example 1: Identification of Candidate Safe Harbor Sites in the Maize Genome

Syngenta elite inbred maize line AX5707 was sequenced and assembled using methods known in the art. The assembled reference genome was annotated using the computer software program MAKER (Cantarel et al. 2008, MAKER: An easy-to-use annotation pipeline designed for emerging model organism genomes. Genome Research. 18(1):188-196. doi:10.1101/gr.6743907), AX5707 mRNA-SEQ data, and information on plant proteins publicly available from Genbank. Sequences intervals which met certain criteria were selected as potential safe harbors for targeted insertion of a DNA of interest. These criteria including: 1) they do not encode genes including predicted gene models, co-localize with mapped RNA-SEQ data or protein coding sequences; 2) they do not encode smRNAs; 3) they are not repeated in the reference genome; 4) they are ≥1,500 bps; 5) they are ≥2,000 bps away from the nearest identified features such as gene models. 61 sequence intervals were identified. (Table 1). The genomic start and stop positions are as identified on the AX5707 reference genome, referred to as MAIZE_JHAX_REG_5. These candidate safe harbor sites were identified as ideal locations in the maize genome for targeted insertion, and may also be referred to as target maize genomic loci for targeted insertion of a DNA of interest.

TABLE 1
Candidate safe harbor sequence intervals in the AX5707 genome

| Chromosome | Genome | Genome end | | SEQ. |
|---|---|---|---|---|
| No. | start position | position | Length (bp) | ID. NO: |
| Chr_1 | 6,051,580 | 6,053,351 | 1,772 | 1 |
| Chr_1 | 9,302,205 | 9,300,245 | 1,961 | 2 |
| Chr_1 | 49,377,512 | 49,375,750 | 1,763 | 3 |
| Chr_1 | 230,909,826 | 230,911,851 | 2,026 | 4 |
| Chr_1 | 233,264,403 | 233,267,221 | 2,819 | 5 |
| Chr_1 | 240,799,470 | 240,801,585 | 2,116 | 6 |
| Chr_1 | 308,952,173 | 308,950,540 | 1,634 | 7 |
| Chr_1 | 325,437,387 | 325,434,968 | 2,420 | 8 |
| Chr_2 | 25,144,470 | 25,146,195 | 1,726 | 9 |
| Chr_2 | 75,055,711 | 75,053,789 | 1,923 | 10 |
| Chr_2 | 76,900,194 | 76,901,853 | 1,660 | 11 |
| Chr_3 | 12,722,668 | 12,724,219 | 1,552 | 12 |
| Chr_3 | 15,938,135 | 15,936,500 | 1,636 | 13 |
| Chr_3 | 58,267,137 | 58,265,554 | 1,584 | 14 |
| Chr_3 | 144,608,280 | 144,610,055 | 1,776 | 15 |
| Chr_3 | 168,663,077 | 168,661,053 | 2,025 | 16 |
| Chr_3 | 187,169,278 | 187,171,380 | 2,103 | 17 |
| Chr_3 | 192,200,564 | 192,199,030 | 1,535 | 18 |
| Chr_3 | 194,535,751 | 194,533,809 | 1,943 | 19 |
| Chr_3 | 199,586,759 | 199,588,327 | 1,569 | 20 |
| Chr_3 | 263,100,502 | 263,098,753 | 1,750 | 21 |
| Chr_4 | 11,109,020 | 11,107,079 | 1,942 | 22 |
| Chr_4 | 11,850,583 | 11,852,381 | 1,574 | 23 |
| Chr_4 | 28,740,222 | 28,741,822 | 1,601 | 24 |
| Chr_4 | 41,128,134 | 41,125,263 | 2,872 | 25 |
| Chr_4 | 122,196,154 | 122,198,795 | 2,642 | 26 |
| Chr_4 | 225,424,622 | 225,422,966 | 1,657 | 27 |
| Chr_4 | 241,199,476 | 241,201,387 | 1,912 | 28 |
| Chr_4 | 241,203,112 | 241,204,386 | 1,275 | 29 |
| Chr_4 | 260,439,892 | 260,441,993 | 2,102 | 30 |
| Chr_5 | 31,399,845 | 31,397,479 | 2,367 | 31 |
| Chr_5 | 52,271,003 | 52,269,062 | 1,942 | 32 |
| Chr_5 | 61,967,477 | 61,969,470 | 1,994 | 33 |
| Chr_5 | 106,828,555 | 106,826,658 | 1,898 | 34 |
| Chr_5 | 194,229,496 | 194,227,080 | 2,417 | 35 |
| Chr_5 | 227,918,248 | 227,916,660 | 1,589 | 36 |
| Chr_5 | 249,991,940 | 249,989,642 | 2,299 | 37 |
| Chr_5 | 254,507,410 | 254,510,012 | 2,603 | 38 |
| Chr_6 | 2,550,820 | 2,549,212 | 1,609 | 39 |
| Chr_6 | 5,357,797 | 5,356,004 | 1,794 | 40 |
| Chr_6 | 102,854,792 | 102,856,689 | 1,898 | 41 |
| Chr_6 | 125,539,340 | 125,536,747 | 2,594 | 42 |
| Chr_6 | 140,569,284 | 140,567,471 | 1,814 | 43 |
| Chr_6 | 172,684,264 | 172,686,334 | 2,071 | 44 |
| Chr_7 | 6,130,641 | 6,133,196 | 2,556 | 45 |
| Chr_7 | 22,848,628 | 22,850,204 | 1,577 | 46 |
| Chr_7 | 92,523,693 | 92,521,688 | 2,006 | 47 |
| Chr_7 | 123,048,334 | 123,046,540 | 1,795 | 48 |
| Chr_7 | 129,393,722 | 129,390,033 | 3,690 | 49 |
| Chr_7 | 143,964,001 | 143,965,742 | 1,742 | 50 |
| Chr_7 | 145,353,967 | 145,352,332 | 1,636 | 51 |
| Chr_7 | 154,264,096 | 154,267,032 | 2,937 | 52 |
| Chr_7 | 172,566,096 | 172,564,156 | 1,941 | 53 |
| Chr_7 | 179,903,048 | 179,901,489 | 1,560 | 54 |
| Chr_7 | 198,992,304 | 198,994,416 | 2,113 | 55 |
| Chr_8 | 30,777,043 | 30,778,622 | 1,580 | 56 |
| Chr_8 | 210,893,628 | 210,891,662 | 1,967 | 57 |
| Chr_9 | 90,581,099 | 90,582,704 | 1,606 | 58 |
| Chr_9 | 137,742,555 | 137,739,791 | 2,765 | 59 |
| Chr_10 | 18,312,696 | 18,314,420 | 1,725 | 60 |
| Chr_10 | 174,905,414 | 174,903,831 | 1,584 | 61 |

### Example 2: Selection of Candidate Maize Genome Safe Harbor Sites for Targeted Insertion

The above identified 61 AX5707 candidate safe harbor sequence intervals were blasted against the publicly available B73 genome (AGPv3/RefGen_v3; available at the maize genetics and genomics database website (Andorf et al., 2016. “MaizeGDB update: new tools, data and interface for the maize model organism database.” Nucleic Acids Res, 44 (dl): D1195-201). Only sequences shared between both B73 and AX5707 genomes with a minimum length of 1,500 bp were selected for further evaluation. Among them, only 1 or 2 of the best candidates from each chromosome were selected. As a result, 10 candidate safe harbor site sequences were selected for experimental validation of targeted insertion. Chromosome 9 and 10 did not have suitable sequence remaining as candidates. The potential safe harbors are described in Table 2. Table 2 indicates the genomic position of the safe harbor in both the AX5707 genome and the B73 genome. The B73 genomic locations are as found in the publicly available MAIZE_B73_REF_4 genome. These target maize genomic loci are particularly useful for targeted insertion of a DNA of interest, because each locus is present in the genome of more than one maize variety. Therefore, the target maize genomic locus is useful for targeted insertion of a DNA of interest into more than one variety of maize cell.

TABLE 2
Candidate safe harbor in AX5707 and B73 genomes

| | | Safe harbor | Genome start | Genome end | Length | SEQ. |
|---|---|---|---|---|---|---|
| Genome | Map | ID | position | position | (bp) | ID. NO: |
| AX5707 | Chr_1 | SH_Chr1.1b | 325,437,387 | 325,434,968 | 2,420 | 8 |
| B73 | Chr_1 | SH_Chr1.1b | 270,485,529 | 270,483,110 | 2,420 | 62 |
| AX5707 | Chr_2 | SH_Chr2.1t | 25,144,470 | 25,146,195 | 1,726 | 9 |
| B73 | Chr_2 | SH_Chr2.1t | 20,384,387 | 20,386,397 | 1715 | 63 |
| AX5707 | Chr_3 | SH_Chr3.1b | 263,100,502 | 263,098,753 | 1,750 | 21 |
| B73 | Chr_3 | SH_Chr3.1b | 218,255,241 | 218,253,476 | 1,766 | 64 |
| AX5707 | Chr_4 | SH_Chr4.1t | 11,850,583 | 11,852,381 | 1,574 | 23 |
| B73 | Chr_4 | SH_Chr4.1t | 11,050,139 | 11,051,738 | 1,600 | 65 |
| AX5707 | Chr_5 | SH_Chr5.1t | 31,399,845 | 31,397,479 | 2,367 | 31 |
| B73 | Chr_5 | SH_Chr5.1t | 26,056,598 | 26,054,232 | 2,367 | 66 |
| AX5707 | Chr_6 | SH_Chr6.1b | 172,684,264 | 172,686,334 | 2,071 | 44 |
| B73 | Chr_6 | SH_Chr6.1b | 152,128,279 | 152,130,349 | 2,071 | 67 |
| AX5707 | Chr_7 | SH_Chr7.1t | 22,848,628 | 22,850,204 | 1,577 | 46 |
| B73 | Chr_7 | SH_Chr7.1t | 20,083,153 | 20,084,729 | 1,577 | 68 |
| AX5707 | Chr_7 | SH_Chr7.2b | 198,992,304 | 198,994,416 | 2,113 | 55 |
| B73 | Chr_7 | SH_Chr7.2b | 172,546,393 | 172,548,490 | 2,098 | 69 |
| AX5707 | Chr_8 | SH_Chr8.1t | 30,777,043 | 30,778,622 | 1,580 | 56 |
| B73 | Chr_8 | SH_Chr8.1t | 25,491,600 | 25,493,179 | 1,580 | 70 |
| AX5707 | Chr_8 | SH_Chr8.2b | 210,893,628 | 210,891,662 | 1,967 | 57 |
| B73 | Chr_8 | SH_Chr8.2b | 178,200,769 | 178,198,798 | 1,972 | 71 |

### Example 3: Construction of CRISPR-Cas9 Expression and Targeting Donor Vectors

The following example describes construction of vectors used for CRISPR-Cas mediated targeted insertion, using the CRISPR-associated site specific nuclease Cas9. It is well-known in the art that there are many different nuclease-mediated targeted insertion systems, including ZFNs, meganucleases, and TALENS. The examples disclosed here do not limit the invention to any particular system of targeted insertion.

To demonstrate that the selected candidate safe harbor sites can be used for targeted insertion, a 20-nucleotide target sequence, which is followed by a 5′-NGG PAM site, within each safe harbor site was chosen for designing a single guide RNA (sgRNA) to test Cas9-mediated cleavage and gene targeting (Table 3). Cas9 and sgRNA-mediated targeted insertion in maize cells have been previously described in the art (WO16106121, herein incorporated by reference). Similar Cas9 and sgRNA expression vector designs were used for testing candidate safe harbor sites here. Each Cas9-sgRNA expression vector (vector ID's shown in Table 3) comprises a coding sequence for a Cas9 nuclease, operably linked to a promoter at its 5′ end and a terminator at its 3′ end, and also comprises a coding sequence for a sgRNA comprising a target sequence which targets the Cas9 to a genomic nuclease cleavage site within the target maize genomic locus, also referred to as the safe harbor. The sgRNA is operably linked at its 5′end to a rice ubiquitin promoter and at its 3′ end to a terminator. The sgRNA for each Cas9-sgRNA expression vector comprises a target sequence (SEQ ID NO: 72-81), as described in Table 3.

TABLE 3
Target sequences for CRISPR-Cas9 targeted insertion

| | | | Target | | |
|---|---|---|---|---|---|
| Safe | Target sequence | Target | sequence | Cas9- | |
| harbor | for Cas9-sgRNA | sequence | SEQ. ID. | sgRNA | Cas9-sgRNA |
| ID | vector design | name | NO. | vector ID | vector Alias |
| SHChr1.1b | 5’-AGCAC CGGTT | xZmSHChr1 | 72 | 23808 | SHChr1_Cas9 |
| | GCTCG GACCG-3’ | | | | |
| SHChr2.1t | 5’-TACAG AAACG | xZmSHChr2 | 73 | 23811 | SHChr2_Cas9 |
| | CGGAG AGACT-3’ | | | | |
| SHChr3.1b | 5’-TAACG AGCAG | xZmSHChr3 | 74 | 23812 | SHChr3_Cas9 |
| | AGTAC ACACG-3’ | | | | |
| SHChr4.1t | 5’-TGAAA GCGAT | xZmSHChr4 | 75 | 23813 | SHChr4_Cas9 |
| | GCGGT TTAGA-3’ | | | | |
| SHChr5.1t | 5’-TACAA TGTAC | xZmSHChr5 | 76 | 23814 | SHChr5_Cas9 |
| | AGTCT AGCCA-3’ | | | | |
| SHChr6.1b | 5’-ACGAG ACCAT | xZmSHChr6 | 77 | 23815 | SHChr6_Cas9 |
| | CCAAT GATCG-3’ | | | | |
| SHChr7.1t | 5’-TGGAG AGTAA | xZmSHChr7a | 78 | 23816 | SHChr7a_Cas9 |
| | TAGGA TGGCA-3’ | | | | |
| SHChr7.2b | 5’-TGAAA CCAAA | xZmSHChr7b | 79 | 23817 | SHChr7b_Cas9 |
| | CCAGC AGACG-3’ | | | | |
| SHChr8.1t | 5’-TAGGT TTGAC | xZmSHChr1 | 80 | 23818 | SHChr8a_Cas9 |
| | ATGTGCTAAG-3’ | | | | |
| SHChr8.2b | 5’-CTTCG TAGAC | xZmSHChr2 | 81 | 23819 | SHChr8b_Cas9 |
| | ATATAGATGC-3’ | | | | |

Donor vectors were also constructed to enable the targeted insertion of a DNA of interest at the target maize genomic locus to be mediated by homologous recombination. The donor vector may also be referred to as the donor molecule. A donor vector which contains at least one homologous “arm” flanking the 5′ and/or the 3′ end of the donor sequence can promote homologous recombination between the arm and the target genomic sequence, thereby leading to targeted insertion by homologous recombination. In these examples, the DNA of interest for each donor vector comprises the coding sequence for the selectable marker phosphomannose isomerase (PMI), which confers an ability to metabolize mannose (U.S. Pat. Nos. 5,767,378 and 5,994,629). The 5′ end of the PMI coding sequence is operably linked to a rice actin promoter and the 3′end of the PMI coding sequence is operably linked to a terminator. Immediately 3′ to the terminator is a homologous arm, comprising the sequence of SEQ ID NO: 82 to 91 for each corresponding safe harbor sequence, as described in Table 4. This homologous arm is sufficient to enable homologous recombination of the donor molecule into the target maize genomic locus at the cleaved genomic nuclease cleavage site. However, it will be appreciated by a person of skill in the art that the homologous arm can be of a different length, and/or that there can be an additional homologous arm adjacent to the DNA of interest at the 5′ end. It will also be appreciated by a person of skill in the art that the presence of the homologous arm(s) enables homologous recombination, however the DNA of interest may still integrate into the genome within the target maize genomic locus by different means, such as non-homologous end joining. These described donor vectors enable CRISPR-Cas9 mediated insertion of the PMI expression cassette at a particular target maize genomic locus by homologous recombination.

As positive controls for targeted insertion, 3 vectors (23813, 23818 and 23819) were constructed for testing intermolecular recombination between donor vectors and the target maize genomic loci (referred to as “target site” in Table 4 below).

TABLE 4
Donor vectors for CRISPR-Cas9 targeted insertion

| | | Targeted | | | Donor vector |
|---|---|---|---|---|---|
| | | insertion | | Safe harbor | safe harbor |
| | Cas9- | donor | | sequence | sequence |
| Safe | sgRNA | vector | Targeted insertion | interval in the | interval SEQ. |
| harbor ID | vector ID | ID | donor vector alias | donor vector | ID. NO. |
| SHChr1.1b | 23808 | 23829 | SHChr1_donor_V2 | xJHAXSHChrl | 82 |
| SHChr2.1t | 23811 | 23828 | SHChr2_donor_V2 | xJHAXSHChr2 | 83 |
| SHChr3.1b | 23812 | 23827 | SHChr3_donor_V2 | xJHAXSHChr3 | 84 |
| SHChr4.1t | 23813 | 23826 | SHChr4_donor_V2 | xJHAXSHChr4 | 85 |
| SHChr5.1t | 23814 | 23825 | SHChr5_donor_V2 | xJHAXSHChr5 | 86 |
| SHChr6.1b | 23815 | 23824 | SHChr6_donor_V2 | xJHAXSHChr6 | 87 |
| SHChr7.1t | 23816 | 23823 | SHChr7a_donor_V2 | xJHAXSHChr7a | 88 |
| SHChr7.2b | 23817 | 23822 | SHChr7b_donor_V2 | xJHAXSChr7b | 89 |
| SHChr8.1t | 23818 | 23821 | SHChr8a_donor_V2 | xJHAXSHChr8a | 90 |
| SHChr8.2b | 23819 | 23820 | SHChr8b_donor_V2 | xJHAXSHChr8b | 91 |

Controls: Intermolecular recombination mediated CRISPR-Cas9

| | | | | | |
|---|---|---|---|---|---|
| 23895, | 23813 | 23826 | SHChr4_donor_V2 | xJHAXSHChr4 | 85 |
| with | | | | | |
| SHChr4V2 | | | | | |
| target site | | | | | |
| 23890 with | 23818 | 23821 | SHChr8a_donor_V2 | xJHAXSHChr8a | 90 |
| SHChr8aV2 | | | | | |
| target site | | | | | |
| 23894 with | 23819 | 23820 | SHChr8b_donor_V2 | xJHAXSHChr8b | 91 |
| SHChr8bV2_ | | | | v2 | |
| target | | | | | |
| site | | | | | |

### Example 4: Targeted Insertion Into Target Maize Genomic Loci in Transiently Transformed Cells

Targeted insertion of the donor PMI expression cassette transgene sequence into different target maize genomic loci mediated by RNA-guided Cas9 cleavage was tested by co-delivering a Cas9-sgRNA expression vector along with the corresponding donor vector (Tables 3 and 4) using particle bombardment, following techniques described previously for targeted insertion into the MIR604 insertion site, which is known to be a good safe harbor site (WO16106121). Briefly, a DNA vector comprising a Cas9-sgRNA expression cassette and a donor vector were precipitated onto gold particles (0.6 μm in diameter; Bio-Rad). A total of 2×10¹⁰ molecules of Cas9-sgRNA expression vector and donor vector at 1:1 ratio were added to a tube of 20 μl prepared gold-glycerol slurry (60 mg/ml) and mixed well by finger tapping. 100 μL of CaCl₂ (2.5 M), and 10 μL of spermidine (0.1 M) were successively added and mixed by vortexing at room temperature. The mixture was then incubated on ice for 30 minutes. The DNA-coated particles were pelleted by centrifuging at 13,000 rpm for 1 minute. After discarding the supernatant, the particles were washed with 200 μL of absolute ethanol by vortexing for 30 seconds, centrifuging for 1 minute, and removing the supernatant and re-suspended in 20 μL of absolute ethanol. For each bombardment, 6 μL of the particle suspension was pipetted onto the center of macrocarriers. Bombardments of 3-day-old pre-cultured immature embryos in osmoticum medium were carried out using a Biolistic particle acceleration device (PDS 1000/He, Bio-Rad) under a chamber pressure of 27.5 mm of Hg at distances of 8, 10, and 65 mm from the rupture disc to the macrocarriers to the stopping screen to the target, respectively, with 1100 psi helium pressures and 3 shots per plates. The combination of Cas9-sgRNA vector, donor vector and numbers of target explants are listed in Table 5. Three days after bombardment, 25 bombarded embryos from each plate were removed for DNA extraction and PCR analysis to determine if targeted insertion of the DNA of interest (PMI expression cassette) into the safe harbor loci in the transformed cells was successful. Two samples were collected for each plate. Positive control experiments with sequences of targeted maize genomic loci cloned into vectors and co-delivered with their respective donors and Cas9-sgRNA expression vectors into maize cells were also performed to assay extra-chromosomal intermolecular recombination.

TABLE 5
Targeted insertion of target genomic loci by CRISPR-Cas9

| | | | | | Explants | # of |
|---|---|---|---|---|---|---|
| | | | | # of | to | mannose |
| Safe | Cas9 | Donor | Total # of | embryos | mannose | resistant |
| harbor ID | vector | vector | embryos | for 1^{st} PCR | selection | callus |
| SHChr1.1b | 23808 | 23829 | 153 | 25 × 2 | 103 | 18 |
| SHChr2.1t | 23811 | 23828 | 134 | 25 × 2 | 84 | 10 |
| SHChr3.1b | 23812 | 23827 | 155 | 25 × 2 | 105 | 35 |
| SHChr4.1t | 23813 | 23826 | 140 | 25 × 2 | 90 | 21 |
| SHChr5.1t | 23814 | 23825 | 121 | 25 × 2 | 71 | 21 |
| SHChr6.1b | 23815 | 23824 | 153 | 25 × 2 | 103 | 27 |
| SHChr7.1t | 23816 | 23823 | 161 | 25 × 2 | 111 | 16 |
| SHChr7.2b | 23817 | 23822 | 150 | 25 × 2 | 100 | 32 |
| SHChr8.1t | 23818 | 23821 | 158 | 25 × 2 | 108 | 13 |
| SHChr8.2b | 23819 | 23820 | 132 | 25 × 2 | 82 | 31 |
| Positive control | 23813 | 23895 | 135 | 25 × 2 | N.A. | N.A. |
| 1: 23895, with | | | | | | |
| SHChr4V2 | | | | | | |
| target site | | | | | | |
| Positive control | 23818 | 23890 | 132 | 25 × 2 | N.A. | N.A. |
| 2: 23890 with | | | | | | |
| SHChr8aV2 | | | | | | |
| target site | | | | | | |
| Positive control | 23819 | 23894 | 121 | 25 × 2 | N.A. | N.A. |
| 3: 23894 with | | | | | | |
| SHChr8bV2 | | | | | | |
| target site | | | | | | |

### Example 5: Molecular Demonstration of Targeted Insertion Into Selected Safe Harbor Loci in Transiently Transformed Cells

Genomic DNA was extracted from bombed maize embryo samples using Promega's Magnesil paramagnetic particles (www.promega.com). PCR primers were designed across the expected 5′ and 3′ homologous recombination junctions for detecting the targeted insertions (Table 6). For each of the expected recombination site, one primer was designed against a genomic sequence of the safe harbor interval, outside the targeted insertion site. The second primer (SEQ ID NO: 105) was designed against a sequence of the donor PMI expression cassette (Table 6). Table 6 also indicates the expected PCR product if targeted insertion was successful. Primers were also designed for the positive controls to assay intermolecular recombination.

TABLE 6
PCR primers for targeted insertion assay

| | SEQ ID NO. | Cas9- | | Expected |
|---|---|---|---|---|
| Safe harbor | of genomic | sgRNA | Donor | PCR product |
| ID | primer | vector ID | vector ID | (bp) |
| SHChr1.1b | 92 | 23808 | 23829 | 1603 |
| SHChr2.1t | 93 | 23811 | 23828 | 1603 |
| SHChr3.1 t | 94 | 23812 | 23827 | 1746 |
| SHChr4.1t | 95 | 23813 | 23826 | 1545 |
| SHChr5.1t | 96 | 23814 | 23825 | 1532 |
| SHChr6.1b | 97 | 23815 | 23824 | 1563 |
| SHChr7.1t | 98 | 23816 | 23823 | 1490 |
| SHChr7.2b | 99 | 23817 | 23822 | 1633 |
| SHChr8.1t | 100 | 23818 | 23821 | 1454 |
| SHChr8.2b | 101 | 23819 | 23820 | 1497 |
| 23895, with | 102 | 23813 | 23826 | 1559 |
| SHChr4V2 | | | | |
| target site | | | | |
| 23890 with | 103 | 23818 | 23821 | 1598 |
| SHChr8aV2 | | | | |
| target site | | | | |
| 23894 with | 104 | 23819 | 23820 | 1513 |
| SHChr8bV2_ | | | | |
| target site | | | | |

To detect targeted insertions in the transiently transformed embryos, two sequential PCR reactions were carried out on each sample to detect potential recombination products using PCR primers designed to amplify across the expected 5′ and 3′ homologous recombination junctions for detecting the targeted insertions (Table 6). The first PCR reaction was setup with 12.5 μl of Sigma JumpStart™ REDTaq ReadyMix™ Reaction Mix, 1 μl of each primers, 4 μl of gDNA and 6.5 μl of H2O. The second PCR reaction was setup with 12.5 μl of Sigma JumpStart™ REDTaq ReadyMix™ Reaction Mix, 1 μl of each primers, 2 μl of the first PCR product as template and 8.5 μl of H2O. PCR was performed on Applied Biosystems Veriti 96 Well Thermal Cycler with following amplification parameters: 95° C. for 5 minutes, 35 cycles of (95° C. 30 seconds, 55° C., 57° C. or 60° C. for 30 seconds as needed and 72° C. for 2 minutes), followed by 7 minutes at 72° C. and then hold at 4° C. until gel electrophoresis. After PCR, 10 μl of PCR product was run on a 1% agarose gel containing SyBR Safe DNA Gel Stain for visualization. PCR products of expected sizes were observed clearly in targeting experiments of 5 safe harbor sites, such that a call could be made regarding the successful targeted integration (Table 7). PCR products were cleaned up with EXO-SAP treatment before sending to Sanger sequence and subjected to Sanger sequencing. Sequencing analysis was carried out using SEQUENCHER™ software, and the sequence data was compared to the reference sequence. These results confirmed successful targeted insertion of the PMI expression cassette into the target genomic loci (SHChr5.1t and SHChr7.1t).

TABLE 7
Analysis of transiently transformed maize embryos for targeted insertion

| | # of | | |
|---|---|---|---|
| Safe | bombed | | Sequence results confirming |
| harbor ID | embryos | PCR results | targeted insertion? |
| SHChr1.1b | 50 | − | No data |
| SHChr2.1t | 50 | + | No |
| SHChr3.1 t | 50 | − | No data |
| SHChr4.1t | 50 | − | No data |
| SHChr5.1t | 50 | + | Yes, confirmed the linkage of PMI |
| | | | to region flanking xJHAXSHChr5 |
| SHChr6.1b | 50 | +/−? | No |
| SHChr7.1t | 50 | + | Yes, confirmed the linkage of PMI |
| | | | to region flanking xJHAXSHChr7a |
| SHChr7.2b | 50 | + | No |
| SHChr8.1t | 50 | − | No data |
| SHChr8.2b | 50 | − | No data |

### Example 6: Targeted Insertion Into Selected Safe Harbor Sites in Stably Transformed Cells

The remaining explants from each plate after sampling at 3 days post-bombardment were transferred onto callus induction media for 10 days. Induced calli were then transferred onto mannose selection media. After 4 weeks of culturing in mannose-contained medium the PMI resistant calli were individually sampled, subjected to DNA extraction and PCR analysis. The numbers of mannose resistant callus were list in Table 8. Genomic DNA was extracted separately from maize embryo and callus tissue. Two sequential PCR reactions were carried out on each sample to detect potential recombination products as described above for transient targeting assays (Table 6 and Table 7). The results of PCR and Sanger sequence analysis are summarized in Table 8. Positive PCR products of expected sizes were found in 6 of 10 tested safe harbor loci. Sanger sequence analysis further confirmed targeted insertion in 3 (SHChr4.1t, SHChr5.1t and SHChr6.1b) out of the 10 target maize genomic loci tested in stably transformed callus tissues. It should be noted that negative PCR or sequencing results do not mean that these safe harbor loci are not amenable to targeted insertion; only very limited experiments were done with each safe harbor locus.

TABLE 8
Analysis of stably transformed tissue for targeted insertion

| Safe harbor | # of calli | | Sequence results confirming |
|---|---|---|---|
| ID | sampled | PCR results | targeted insertion? |
| SHChr1.1b | 18 | no positive | No data |
| SHChr2.1t | 10 | 6 positive | No |
| SHChr3.1 t | 35 | 1 positive | No |
| SHChr4.1t | 21 | 1 positive | Yes, confirmed the linkage of PMI |
| | | | to region flanking xJHAXSHChr4 |
| SHChr5.1t | 21 | 1 positive | Yes, confirmed the linkage of PMI |
| | | | to region flanking xJHAXSHChr5 |
| SHChr6.1b | 27 | 1 positive | Yes, confirmed the linkage of PMI |
| | | | to region flanking xJHAXSHChr6 |
| SHChr7.1t | 16 | 12 positive | No |
| SHChr7.2b | 32 | No positive | No data |
| SHChr8.1t | 14 | No positive | No data |
| SHChr8.2b | 31 | No positive | No data |

1. A method of integrating a DNA of interest into a target maize genomic locus in a maize genome, wherein the target maize genomic locus comprises a nucleic acid sequence having at least 80% sequence identity to at least 100 contiguous nucleotides of SEQ ID NO: 1-71 or the complement thereof, comprising introducing into a maize cell:
a) a first nucleic acid molecule comprising at least 100 contiguous nucleotides with at least 80% identity to at least 100 contiguous nucleotides of SEQ ID NO: 1-71 or the complement thereof, and further comprising the DNA of interest; and
b) a second nucleic acid molecule comprising a nucleotide sequence encoding a nuclease for site-directed cleavage at a genomic nuclease cleavage site within the target maize genomic locus,
under conditions wherein expression of the second nucleic acid molecule can occur to produce the nuclease and the nuclease can cleave the nucleotide sequence at the genomic nuclease cleavage site, whereby the DNA of interest is integrated into the target maize genomic locus in the maize genome.

2. The method of claim 1, wherein two or more DNA of interest are inserted into two or more targeted maize genomic loci.

3. The method of claim 1, wherein the DNA of interest inserted into the target maize genomic locus via homologous recombination.

4. The method of claim 1, wherein the DNA of interest inserted into the target maize genomic locus via non-homologous end-joining.

5. The method of claim 1, wherein the DNA of interest and/or the target maize genomic locus are modified during insertion of said DNA of interest into said target maize genomic locus.

6. A method of producing a maize plant, plant part, or progeny thereof comprising a DNA of interest, comprising regenerating a maize plant from the maize cell produced by the method of claim 1.

7. A maize plant, plant part, or progeny thereof comprising a DNA of interest, produced by the method of claim 6.

8. A method of making a maize plant cell comprising a DNA of interest, said method comprising:
a. selecting a target maize genomic locus, wherein the target maize genomic locus comprises a nucleic acid sequence having at least 80% sequence identity to a sequence selected from the group consisting of SEQ ID NO: 1-71, and the complement thereof;
b. selecting a site specific nuclease that specifically binds to and cleaves a genomic nuclease cleavage site within said target maize genomic locus;
c. introducing said site specific nuclease and a DNA of interest into the maize plant cell;
d. allowing the DNA of interest to insert into the target maize genomic locus; and
e. selecting maize plant cells that comprise the DNA of interest inserted into the target maize genomic locus.

9. The method of claim 8, wherein the target maize genomic locus comprises a nucleic acid sequence with at least 80% sequence identity to a sequence selected from the group consisting of SEQ ID NO: 1, 8, 9, 13, 21, 22, 23, 31, 37, 39, 40, 44, 46, 55, 56, 57, 62-71, and the complement thereof.

10. The method of claim 9, wherein two or more DNAs of interest are inserted into two or more targeted maize genomic loci.

11. The method of claim 9, wherein the DNA of interest is inserted into the target maize genomic locus via homologous recombination.

12. The method of claim 9, wherein the DNA of interest is inserted into the target maize genomic locus via non-homologous end-joining.

13. The method of claim 9, wherein the DNA of interest and/or the target maize genomic locus are modified during insertion of said DNA of interest into said target maize genomic locus.

14. The method of claim 9, wherein the site specific nuclease is a Cas-associated nuclease and wherein a third nucleic acid molecule encoding a guide RNA is introduced into the maize cell.

15. The method of claim 9, wherein the site specific nuclease is a Cas-associated nuclease and wherein a third nucleic acid molecule comprising a guide RNA is introduced into the maize cell.

16. A method of producing a maize plant or plant part, or progeny thereof, comprising a transgene integrated into a genomic nuclease cleavage site in the maize genome, comprising regenerating a maize plant from the maize cell produced by the method of claim 9.

17. A maize plant or plant part, or progeny thereof, comprising a transgene integrated into a genomic nuclease cleavage site in the maize genome, produced by the method of claim 16.

18. A maize recombinant molecule, wherein the recombinant molecule comprises a nucleic acid sequence of at least 100 nucleotides and has at least 80% sequence identity to at least 100 contiguous nucleotides of SEQ ID NO: 1-71 or the complement thereof, and wherein the recombinant molecule further comprises a DNA of interest, wherein the DNA of interest is inserted into the nucleic acid sequence to produce said recombinant molecule.

19. The recombinant molecule of claim 18, wherein the recombinant sequence comprises a nucleic acid sequence of at least 1 Kb and has at least 80% sequence identity to a sequence selected from the group consisting of SEQ ID NO: 1, 8, 9, 13, 21, 22, 23, 31, 37, 39, 40, 44, 46, 55, 56, 57, 62-71, and the complement thereof.

20. A maize plant, plant part, or plant cell comprising the recombinant molecule of claim 18.
